(19) United States
(12) Patent Application Publication
(10) Pub. No.: US 2023/0158153 A1
(43) Pub. Date: May 25, 2023

(54) PI3 KINASE INHIBITORS AND USES THEREOF

(71) Applicant: Purdue Research Foundation, West Lafayette (IN)

(72) Inventors: Philip Stewart Low, West Lafayette, IN (US); Madduri Srinivasarao, West Lafayette, IN (US)

(21) Appl. No.: 17/995,806

(22) PCT Filed: Apr. 9, 2021
(86) PCT No.: PCT/US2021/026691

§ 371 Date: Oct. 7, 2022

Related U.S. Application Data
(60) Provisional application No. 63/007,847, filed on Apr. 9, 2020.

(51) Int. Cl. A61K 47/54; A61P 11/00; C07D 401/14; C07D 401/04

(52) U.S. Cl. CPC A61K 47/545; A61P 11/00; C07D 401/14; C07D 401/04; A61K 47/542

(57) ABSTRACT

A compound of the formula (II); a pharmaceutical composition comprising same; and methods for treating a fibrotic disease in a subject.

### CROSS REFERENCE TO RELATED APPLICATIONS

This application claims priority to U.S. provisional patent application No. 63/007,847, which was filed on Apr. 9, 2020, and which is hereby incorporated by reference in its entirety,

### INCORPORATION BY REFERENCE OF SEQUENCE LISTING

A Sequence Listing is provided herewith as a text file, “21291.06.txt” created on Mar. 30, 2021 and having, a size of 4,096 bytes. The contents of the text file are incorporated by reference herein in their entirety.

### BACKGROUND

Pathologic fibrosis involves the excessive deposition of fibrous tissue, primarily collagen, leading to tissue remodeling that interferes with normal organ function and ultimately leads to organ failure. Although virtually any tissue can experience pathologic fibrosis, the most commonly affected organs are the lungs, kidneys, liver, skin, heart, and bladder. Owing to the difficulty in diagnosing these diseases, their total incidences have not been accurately recorded; however, it has been estimated that 30-40% of morbidity in developed countries is caused by their collective occurrence.

Idiopathic pulmonary fibrosis (IPF) arises from progressive fibrosis of the lungs that occurs primarily in individuals over the age of 50 and commonly results in death within 3-5 years of diagnosis. In the US, IPF kills ˜40,000 people/year (i.e., as many as breast cancer), with most treatment options focused on managing patient lifestyle and/or supplementing oxygen supply.

Although two drugs, pirfenidone and nintedanib, have been approved for treatment of IPF, both provide only limited and inconsistent efficacy, primarily retarding disease progression but not leading to resolution of the pathology. Several kinase inhibitors have also been introduced into clinical trials; however, their inhibition of the targeted enzymes in healthy tissues has raised concerns regarding possible systemic toxicities. And although lung transplantation remains a final treatment option, survival is still often limited, and the cost of lung transplantation is high compared to medical therapies.

Given these drawbacks, there is a need for improved approaches for the treatment of fibrosis. It is an object of the present disclosure to provide such an approach. This and other objects and advantages, as well as inventive features, will be apparent from the description. provided herein.

### SUMMARY

The disclosure relates to a compound of the formula (I):

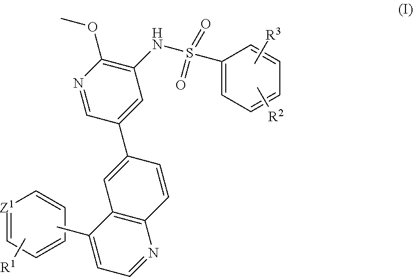

or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R¹ is hydroxyalkyl, aminoalkyl, —S(O)ₓalkyl (wherein x is 0, 1 or 2), carboxyl, carboxylalkyl, thiocarboxyl, thiocarboxylalkyl, amino or amidoalkyl;
R² and R³ are each, independently, H, halo, hydroxy, alkyl, alkoxy, aryl, amino, acyl or C(O)R^{b}, wherein R^{b} is alkyl, aryl, OH or alkoxy.

The disclosure also relates to a compound of formula (II):

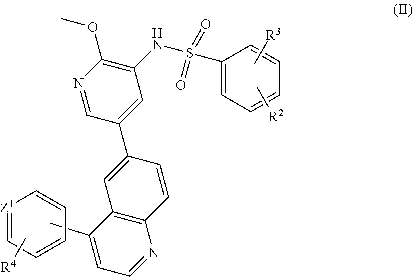

or a pharmaceutically acceptable salt thereof wherein;
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R⁴ is a group of the formula D-L-O-alkyl-, D-L-N(R^{e})-alkyl-, D-L-S(O)ₓalkyl, D-L-C(O)—, or D-L-C(O)-alkyl, wherein L is a linker, D is a fibroblast activation protein (FAP) ligand, R^{e} is H or alkyl, and x is x is 0, 1, or 2; and
R² and R³ are each, independently, H, halo, hydroxy, alkyl, alkoxy, aryl, amino, acyl or C(O)R^{b}, wherein R^{b} is alkyl, aryl, OH or alkoxy.

The compound can be a compound of the formula:

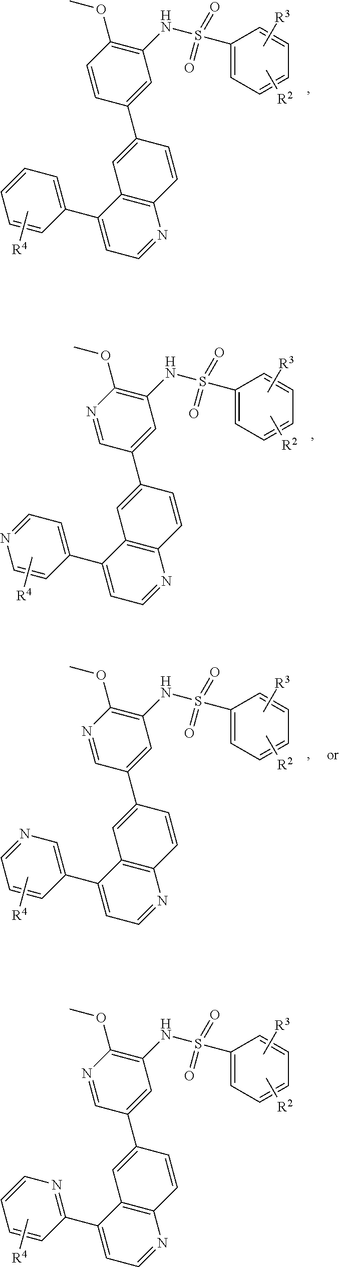

or a pharmaceutically acceptable salt thereof.

The compound can be a compound of the formula:

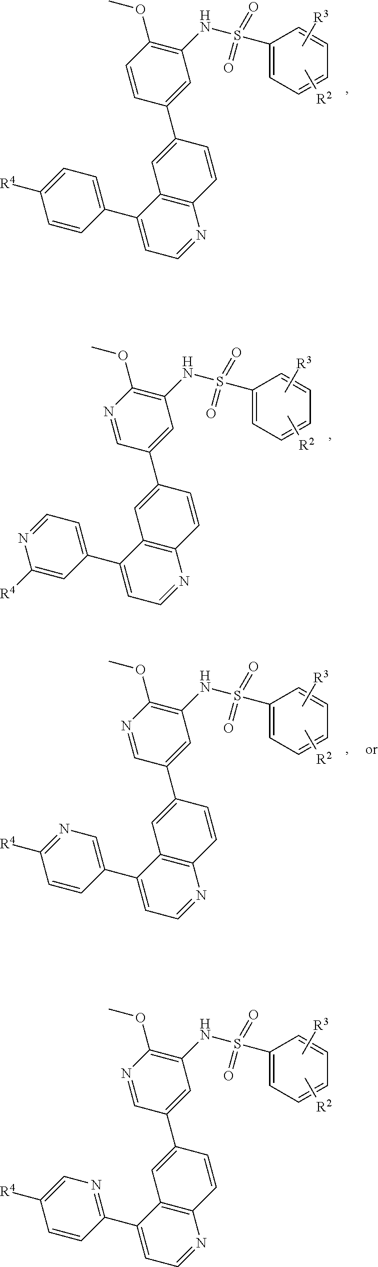

or a pharmaceutically acceptable salt thereof.

L can be a hydrolyzable linker. L can be an optionally substituted heteroalkyl. The substituted heteroalkyl can be substituted with at least one substituent selected from the group consisting of alkyl, hydroxyl, acyl, polyethylene glycol (PEG), carboxylase, and halo. L can be a substituted heteroalkyl with at least one disulfide bond in the backbone thereof. L can be a peptide or a peptidoglycan with at least one disulfide bond in the backbone thereof. L can be of the formula:

—CO—(CH₂)₂—CONH—CH(COOH)—CH₂—CR⁶R⁷—S—S—CH₂—O—CO—,

wherein R⁶ and R⁷ are each, independently, H, alkyl, or heteroalkyl. L can be a group or can comprise a group of the formula:

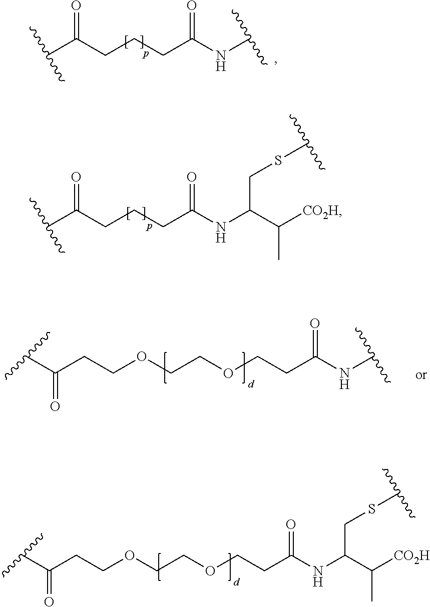

wherein p is an integer from 0 to 10; and d is an integer from 1 to 40.

D can be a group or can comprises a group of the formula (III):

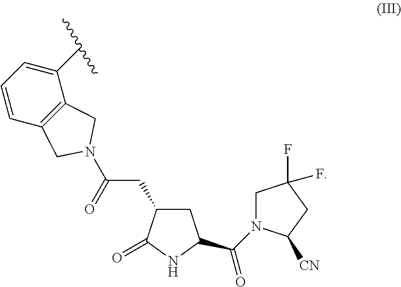

D can be a group or can comprise a group of the formula (IV):

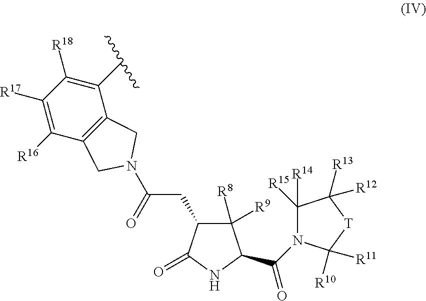

wherein,
T is CH₂, NH, O or S;
R¹⁰ and R¹¹ are each, independently, —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F or 5-tetrazolyl;
R¹² and R¹³ are each, independently, —H, —OH, F, Cl, Br, I, —C₁₋₆alkyl, —O—C₁₋₆alkyl, or —S—C₁₋₆alkyl;
R⁸, R⁹, R¹⁴, and R¹⁵ are each, independently, H, alkyl or halo; and
R¹⁶-R¹⁸ are each, independently, H, —C₁₋₆alkyl, —O—C₁₋₆alkyl, —S—C₁₋₆ alkyl, F, Cl, Br, or I.

D can be a group or can comprise a group of the formula (V):

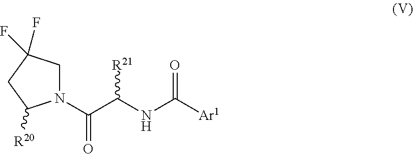

wherein,
R²⁰ is —H, —CN, —B(OH)₂, —C(O)alkyl, —C(O)aryl, —C═C—C(O)aryl, —C═C—S(O)₂aryl, —CO₂H, —SO₃H, —SO₂NH₂, —PO₃H₂, or 5-tetrazolyl;
R²¹ is H or CH₃; and
Ar¹ is substituted phenyl, pyridyl, chloropyridyl, or quinolinyl.

The compound of the formula (II) can be a compound of the formula:

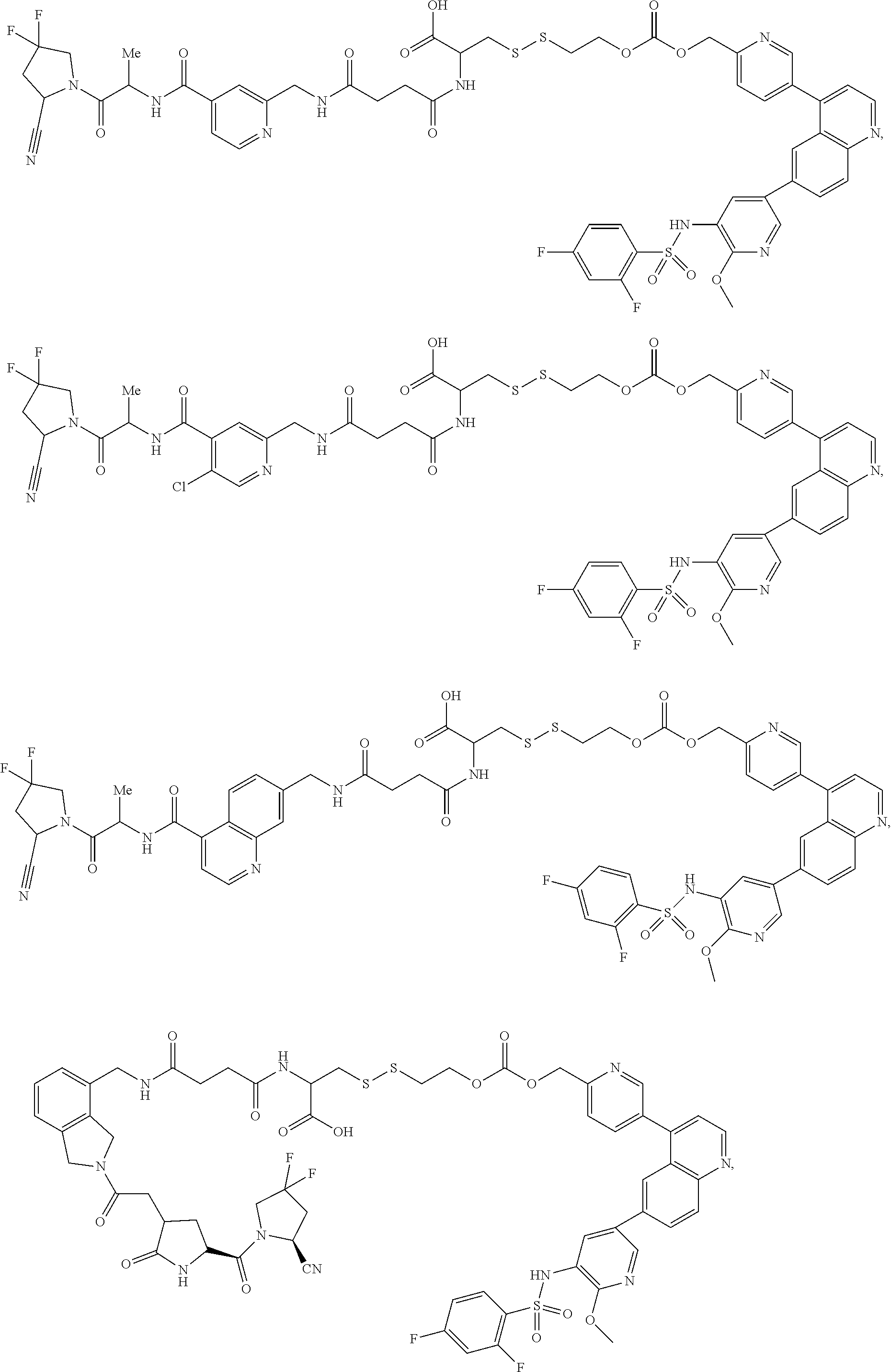

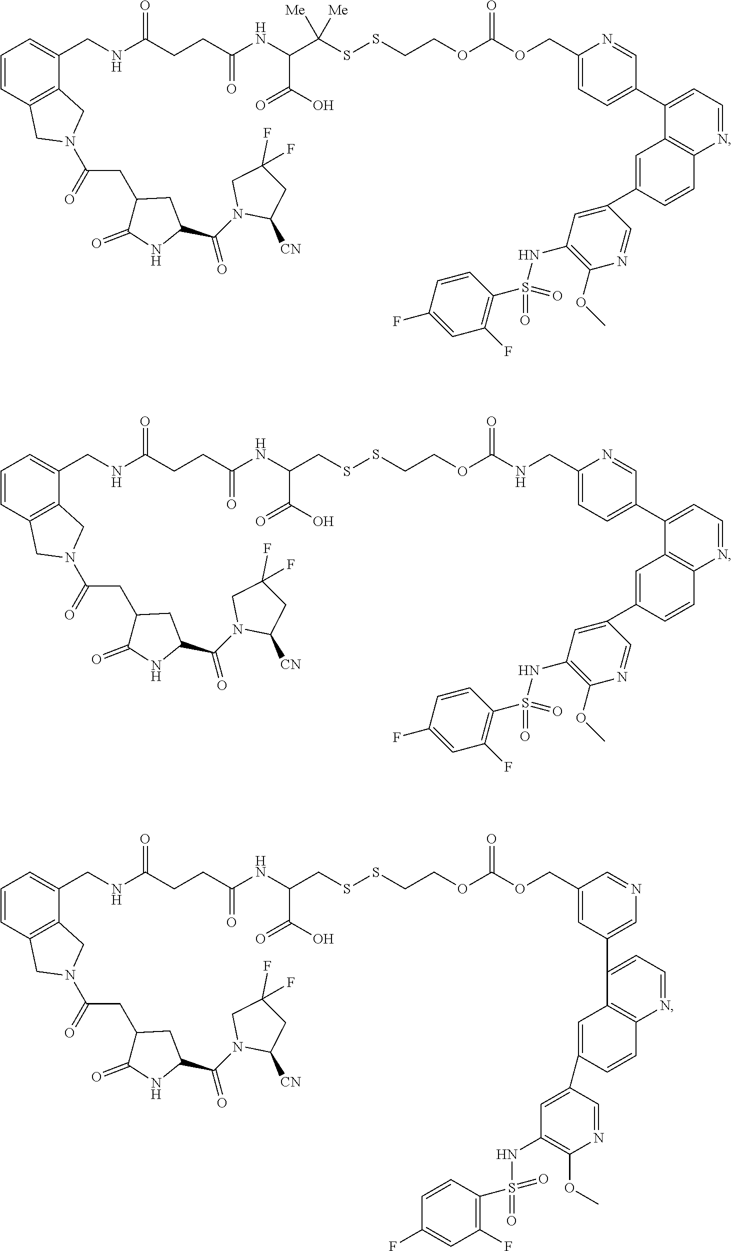

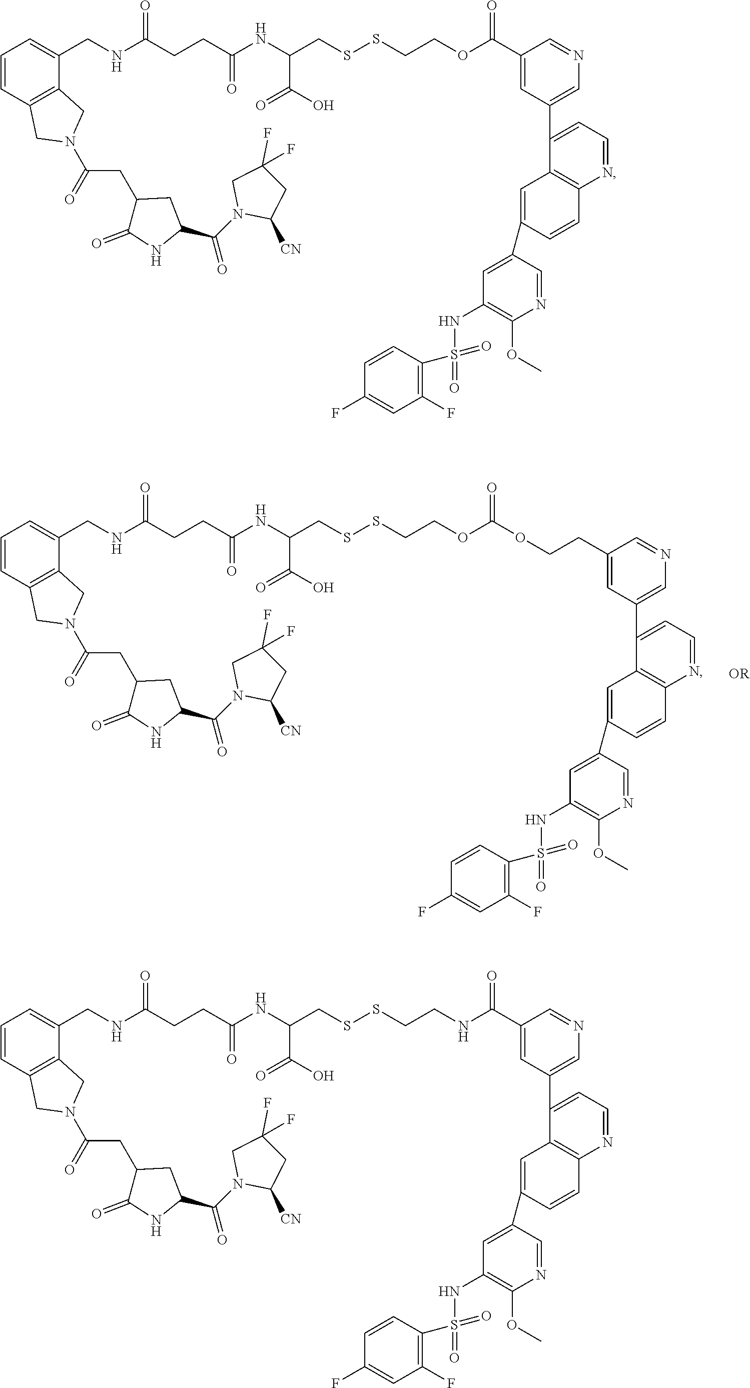

or a pharmaceutically acceptable salt thereof.

The disclosure also relates to a pharmaceutical composition comprising a therapeutically effective amount of one or more of the above compounds and at least one pharmaceutically acceptable excipient.

The disclosure also relates to a method for treating fibrosis, the method comprising administering a therapeutically effective amount of one or more compounds or a pharmaceutical composition to a subject in need thereof.

### DESCRIPTION OF THE DRAWINGS

FIG. 1 shows flow cytometric analysis of human lung tissue samples showing upregulation of FAP only on interstitial pulmonary fibrosis (IPF) lung fibroblasts.

FIG. 2A shows structures of FAP ligand-targeted fluorescein (FAPL-fluorescein), FAPL-PI-3 kinase inhibitor (FAPL-PI3Ki1), and FAPL-S0456 (a near-infrared dye) (FAPL-S0456).

FIG. 2B shows cell images of FAPL-fluorescein binding and internalization. Confocal microscopy of human lung fibroblasts expressing fibroblast activation protein (HLF-FAP cells) expressing the late endosomal marker Rab7a conjugated with red fluorescent protein (Rab7a-RFP) incubated with FAPL-fluorescein and imaged 5 min (a-c) and 30 min (d-f) after FAPL-fluorescein addition. FAPL-fluorescein staining is shown in green, while Rab7a-RFP staining is shown in red. DRAQ5 nuclei staining is shown in blue. Colocalization of FAPL-fluorescein with Rab7a-RFP is shown in panels c and f (indicated in yellow).

FIG. 3 shows FAPL-fluorescein (nM) binding to HLF cells before and after transfection with human FAP (hFAP).

FIG. 4 shows FAP-Fluorescein can bind FAP on fibroblasts from an IPF patient. Comparison of FAPL-Fluorescein uptake by non-IPF (upper row; control) and IPF (lower row) HFL. Binding of FAPL-fluorescein and expression of alpha smooth muscle actin (αSMA), a fibroblast activation marker, are shown in green and red, respectively. The merging of the two markers is shown in pink (right column).

FIG. 5A is the structure of omipalisib, a potent PI3K inhibitor in human clinical trials.

FIG. 5B is the structure of the derivatizable analog of omipalisib, PI3Ki, for use in conjugation via a releasable linker to FAPL.

FIG. 5C is a schematic showing the release of PI3Ki upon cell entry. The reductive environment of the endosome cleaves the disulfide bond, triggering a self-immolative release of the free PI3Ki. (PI3Ki1).

FIG. 5D is a Schrödinger Maestro docking of the pan-PI-3-Kinase/mTOR inhibitor (omipalisib; left panel), pyridine-hydroxymethyl derivative of omipalisib (PI3Ki1; center panel) and overlay of the two inhibitors (right panel) in the active site of PI3Kγ (PDB code: 3L08).

FIG. 6A shows Western blots of confluent HLF stimulated with TGFβ1 (10 ng/mL) and treated with the indicated concentrations of either PI3Ki1or omipalisib. Lysates were collected and analyzed for the indicated proteins by Western blotting, wherein pAkt (S473) is phosphorylated protein kinase B, Akt is protein kinase B, Col.1 is collagen 1, αSMA is α-smooth muscle actin, pSMAD2 is phosphor-SMAD2 kinase, SMAD2 is mothers against decapentaplegic homolog 2, and GAPDH is glyceraldehyde 3-phosphate dehydrogenase.

FIG. 6B shows quantitation of the impact of increasing concentrations of PI3Ki1 or omipalisib an the ratio of Col.1/GAPDH in the same TGF β1-stimulated HLF cells.

FIG. 6C shows quantitation of the effect of increasing concentrations of PI3Ki1or omipalisib on the ratio of pAkt/Akt in TGFβ1-stimulated HLF cells.

FIG. 6D shows the effect of 100 nM PI3Ki1 or omipalisib on the ability of HLF to induce contraction of a collagen gel (collagen contraction is characteristic of activated fibroblasts) compared to control and untreated HLF. Data were analyzed using one-way ANOVA, followed by post-hoc Tukey test (n=3; *p<0.01).

FIG. 6E shows the effect of increasing concentrations of PI3Ki1or omipalisib on caspase 3 and 7 activities in HLF cells as a measure of drug-induced apoptosis. The experiments in panels A-E have been reproduced three times, each with three independent samples, n=3).

FIG. 7A shows the densitometric quantification of the effect on the pAkt/Akt1 ratio of stimulation of confluent human IPF fibroblasts with TGFβ1 (10 ng/mL,) and subsequent treatment with increasing concentrations of FAPL-PI3Ki1, with or without excess FAPL (10th), for 2 hours. After replacing the medium with inhibitor-free medium, the cells were cultured for an additional 22 hours, lysed with phosphatase inhibitor containing cell lysis solution, and analyzed for the indicated proteins by Western blotting (n=3).

FIG. 7B shows the densitometric quantification of the effect on the pAkt/Akt1 ratio of treatment of confluent IPF fibroblasts with FAPL-PI3Ki1 or nontargeted PI3Ki1 for 3 min, 9 min, 27 mm, or 81 min, after which the media were replaced with TGFβ1-containing (10 ng/mL) media lacking PI3Ki. After an additional 24-hour incubation, cells were lysed and the indicated proteins were analyzed by Western blotting (n=3).

FIG. 7C shows representative Western blots showing the impact of FAP knockdown with FAP shRNA (shFAP) on the efficiency of FAPL-PI3Ki1 suppression of Akt phosphorylation. Random/zed shRNA (shCTL) served as a control (n=2).

FIG. 7D shows a representative Western blot showing the efficacy of FAP knockdown with shFAP and shCTL in IPF fibroblasts and the densitometric quantification of FAP knockdown in these blots (n=3).

FIG. 7E and FIG. 7F show assay of collagen biosynthesis (green channel) using a molecular crowding assay (0.1% DMSO vehicle was constant for all experimental conditions). IPF fibroblasts were treated with 100 nM omipalisib, PI3Ki1 or FAPL-PI3Ki1for 2 hours, after which the media were removed and the fibroblasts were further stimulated for 48 h with media containing TGFβ1 (10 ng/mL). Cell counts were obtained from DAPI counterstaining (blue channel). Data were analyzed using one-way ANOVA, followed by post-hoc Tukey test (n=3; *p.-(0.05).

FIG. 7G shows representative Western blots of confluent human IPF fibroblasts stimulated with TGFβ1 (10 ng/mL) and treated with the indicated concentrations of FAPL-PI3Ki1. Lysates were collected and analyzed for the indicated proteins or phosphoproteins (indicated by “p”) by Western blotting. Akt is a substrate of PI3K, 4E-BP1 is a substrate of mTOR, and S6 is a substrate of a kinase (S6 kinase) that is activated by mTOR.

FIGS. 8A-8B are representative Western blots showing that FAP-targeted PI-3 Kinase inhibitor (FAPL-PI3Ki1) suppresses phosphorylation of Akt in IPF fibroblasts.

FIG. 9A shows representative optical images of whole body (upper panel) and tissue biodistribution (lower panel) of a FAPL-targeted near infrared fluorescent dye (FAPL-S0456) 3 hours following its intravenous administration into mice with Bleo-induced lung fibrosis. Note that little or no FAPL-S0456 is retained in any tissue except the fibrotic lungs, and this lung uptake is both blocked by excess FAPL (right panel) and absent from healthy mice (left panel), i.e., demonstrating the specificity of FAPL-S0456 for the fibrotic lung. The time course of fibrosis in this model is shown in panels B-D.

FIG. 9B shows changes in lung tissue density and bronchio-centric scarring (see arrows in images on days 7 and 14 following intratracheal administration of 0.75 μg/Kg Bleo).

FIG. 9C shows images of lung uptake of FAPL-S0456 over the same time course as in FIG. 9B, and its quantitation in FIG. 9D.

FIG. 9D is a quantitation of the image of lung uptake of FAPL-S0456 over the same time course as in FIG. 9B: n=5 for the healthy group, n=5 for the Day 7 group, n=5 for the Day 14, and n=5 for the Day 21 group. Data were analyzed using one-way ANOVA, followed by post-hoc Tukey test (*p<0.05).

FIG. 10A is a schematic representation of the experimental protocol for induction, treatment and therapeutic intervention in a bleomycin-induced lung fibrosis model in mice.

FIG. 10B shows changes in body weights of healthy, FAPL-PI3Ki1 (green) and vehicle (red) treated mice.

FIG. 10C shows the survival of FAPL-PI3Ki1-treated and vehicle-treated mice relative to healthy controls.

FIG. 10D shows the hydroxyproline content (μg/right lung) on day 21 of healthy and fibrotic mice following treatment with or without FAPE-PI3Ki1. Hydroxyproline data are displayed as box plots, with the band inside the box representing the mean, and the whiskers representing the minimum and maximum values.

FIG. 10E is Masson trichrome staining of excised lung sections from healthy mice and Bleo-treated mice obtained following treatment with FAP-PI3Ki1or vehicle (control).

FIGS. 10F and 10G show Western blots showing α-SMA expression in lungs of different group of mice and densitometric quantification of the α-SMA/β-Actin ratio.

FIG. 10H shows the ratio of collagen 1A1/18s expression in lungs of different group of mice.

FIG. 10I shows Western blot analysis of phosphorylated Akt (pAkt(S473) and total Akt in the lung cell lysates from the contralateral lungs of the same mouse cohorts.

FIG. 10J is the ratio of pAkt/Akt in the two different treatment groups. For the pAkt Western blot study, n=3 for the vehicle treated group, and n=4 for FAPL-PI3Ki1treated group. For the different groups in the therapy study, n=5 for the healthy group, n=10 for the FAPL-PI3Ki1 group, and n=10 for the vehicle group. Data were analyzed using one-way ANOVA, followed by post hoc Tukey test (*p<0.05).

FIG. 11A shows the results of the evaluation of a panel of PI3K-mTor inhibitors.

FIG. 11B is the RT-PCR graph of some PFK-mTor inhibitors.

### DETAILED DESCRIPTION

Described herein are the synthesis and use of fibroblast activation protein (FAP)-specific targeting ligands for the delivery of a phosphatidylinositol 3-kinase (PI3K) inhibitor to collagen-producing fibroblasts in fibrotic lung tissues. The FAP-targeted MK inhibitors can inhibit PI3K activity in both normal lung fibroblasts activated with TGFβ1 and human interstitial pulmonary fibrosis (IPF) lung fibroblasts cultured in vitro. Further, the FAP-targeted inhibitors can suppress alpha smooth muscle actin expression (αSMA; a marker of fibroblast activation), hydroxyproline production (a building block of collagen), collagen deposition, and development of lung fibrosis in mice induced to develop experimental lung fibrosis with bleomycin. Lung slices from human IPF patients respond similarly to treatment with the FAP-targeted PI3K inhibitors.

While the concepts of the present disclosure are illustrated and described in detail in the figures and descriptions herein, results in the figures and their description are to be considered as examples and not restrictive in character; it being understood that only the illustrative embodiments are shown and described and that all changes and modifications that come within the spirit of the disclosure are desired to be protected.

### Compounds

Provided are compounds of the formula (I):

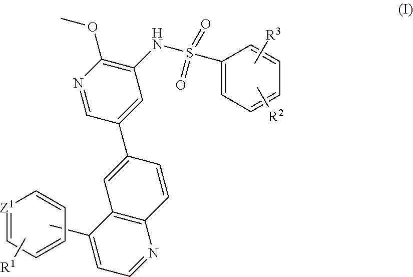

or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R¹ is hydroxyalkyl, aminoalkyl, —S(O)ₓalkyl (wherein x is 0, 1 or 2), carboxyl, carboxylalkyl, thiocarboxyl, thiocarboxylalkyl, amido or amidoalkyl;
R² and R³ are each, independently, H, halo, hydroxy, alkyl, alkoxy, aryl, amino, acyl or C(O)R^{b}, wherein R^{b} is alkyl, aryl, OH or alkoxy.

Examples of compounds of the formula (I) include compounds of the formulae:

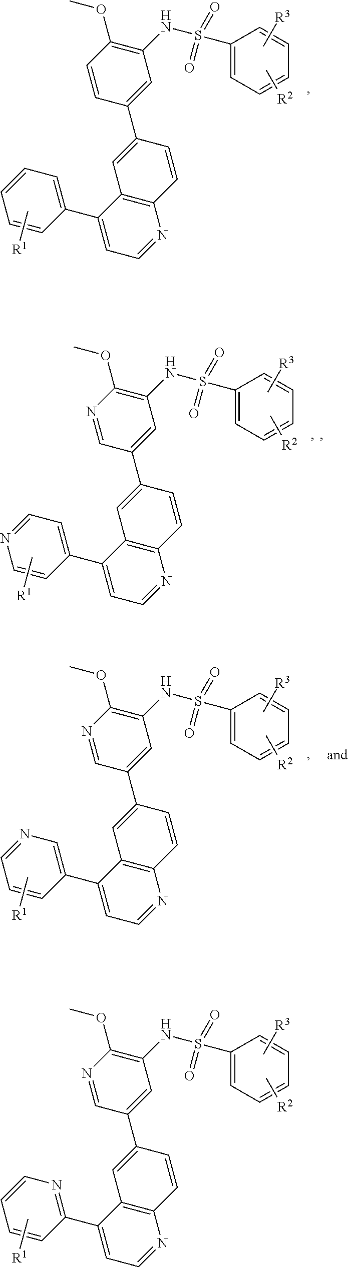

or a pharmaceutically acceptable salt thereof.

Examples of compounds of the formula (I) also include compounds of the formulae:

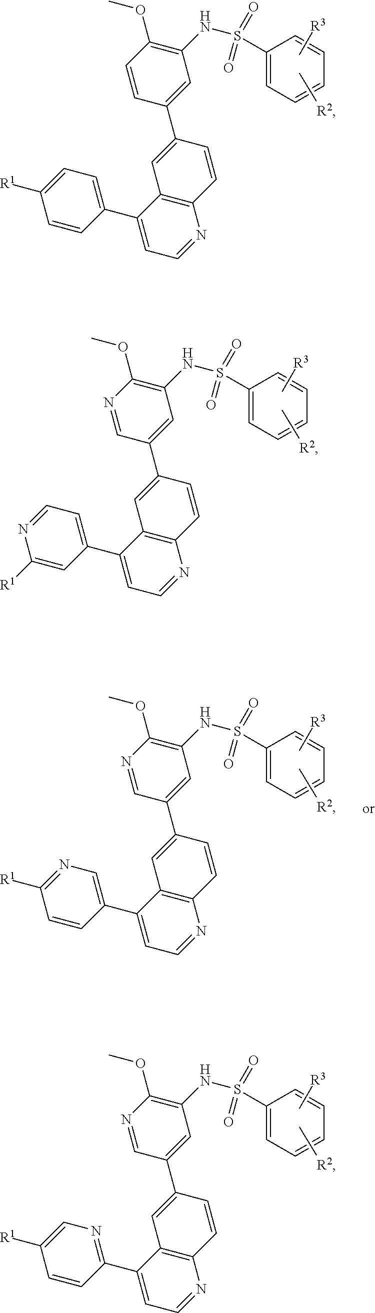

or a pharmaceutically acceptable salt thereof.

Examples of R¹ groups that can be present on any of the compounds described herein include groups of the formula R^{c}O-alkyl- (e.g., R^{c}O(CH₂)ₙ—, wherein R^{c} is H or a hydroxyl protecting group; groups of the formula (R^{d})₂N-alkyl- (e.g., (R²)₂N(CH₂)ₙ—), wherein R^{d} is H or an amine protecting group; R^{e}S(O)ₓ-alkyl- (e.g., R^{e}S(O)ₓ(CH₂)ₙ—), wherein R^{e} is H or alkyl, and x is 0, 1, or 2; R^{e}O(O)C—, wherein R^{e} is H or alkyl; R^{e}O(O)C-alkyl- (e.g., R^{e}O(O)C(CH₂)ₙ—), wherein R^{e} is H or alkyl; R^{e}S(O)C—, wherein R^{e} is H or alkyl; R^{e}S(O)C-alkyl- (e.g., R^{e}S(O)C(CH₂),-), wherein R^{e} is H or alkyl; (R^{e})₂N(O)C—, wherein R^{e} is H or alkyl; and (R^{e})₂N(O)C-alkyl- (e.g., (R^{e})₂N(O)C(CH₂)ₙ—), wherein R^{e} is H or alkyl. In any of these R¹ groups, n can be an integer from 1 to 20 (e.g., 1 to 10, 2 to 5, 3 to 10, 5 to 15, and 1 to 5).

Compounds of the formula (I) include compounds of the formulae:

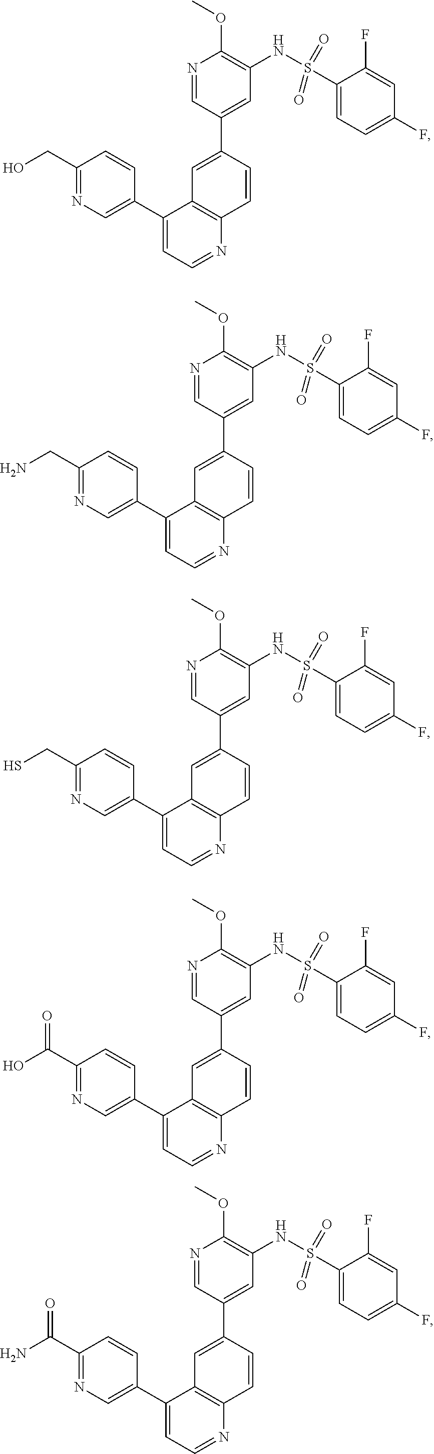

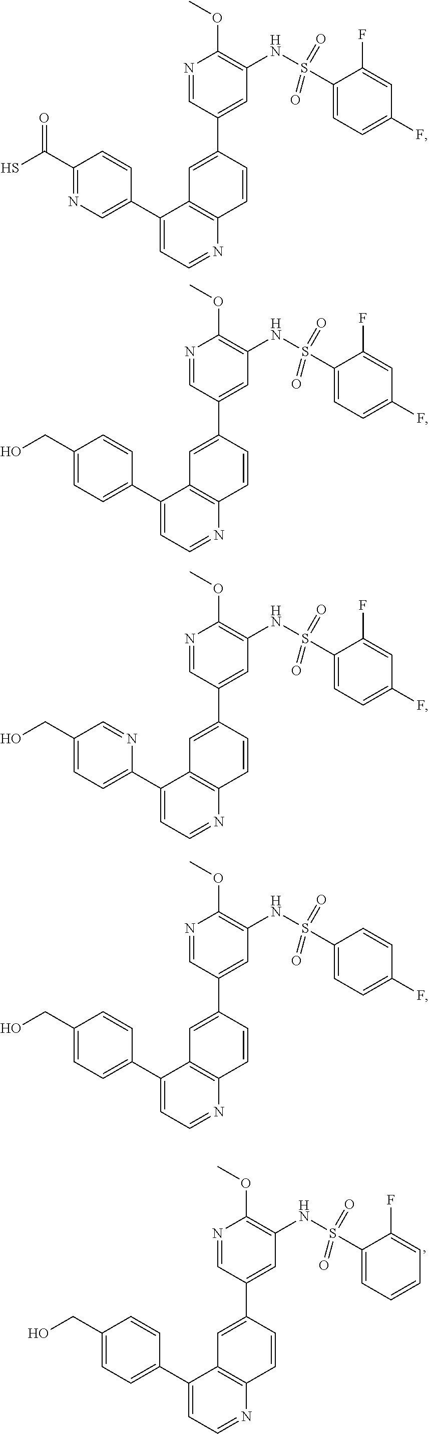

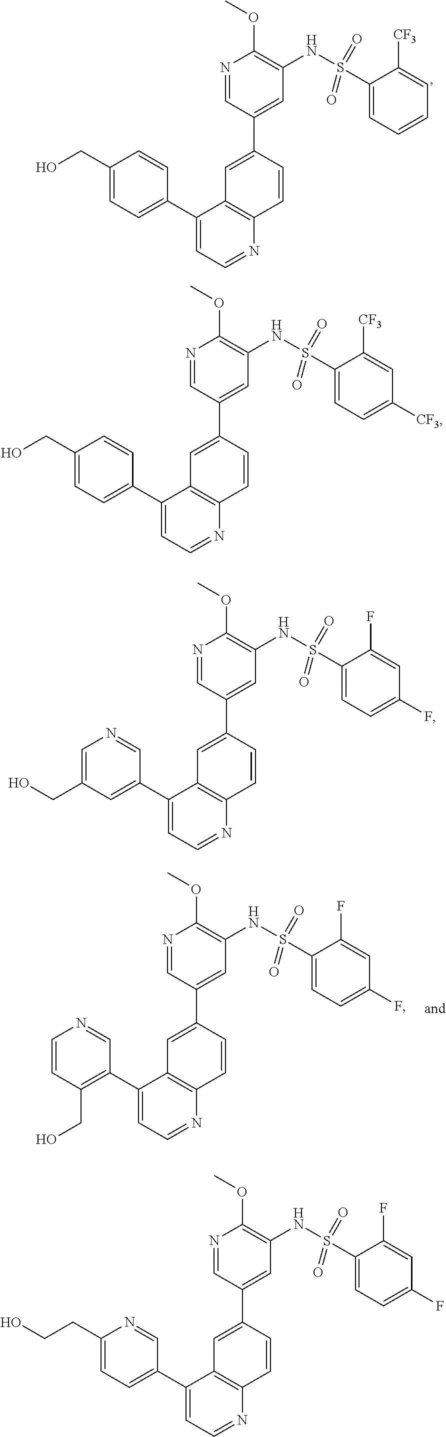

or a pharmaceutically acceptable salt thereof.

The instant disclosure also relates to compounds of the formula (II):

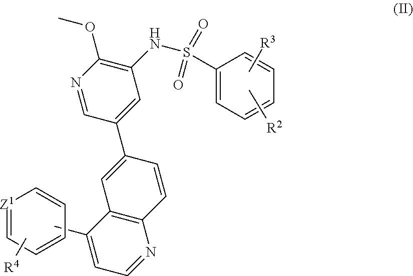

or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R⁴ is a group of the formula D-L-O-alkyl-, D-L-N(R^{e})-alkyl-, D-L-S(O)ₓalkyl, D-L-C(O)—, or D-L-C(O)-alkyl, wherein L is a linker, and D is a FAP ligand; and
R² and R³ are each, independently, H, halo, hydroxy, alkyl, alkoxy, aryl, amino, acyl or C(O)R^{b}, wherein R^{b} is alkyl, aryl, OH or alkoxy.

Examples of compounds of the formula (II) include compounds of the formulae:

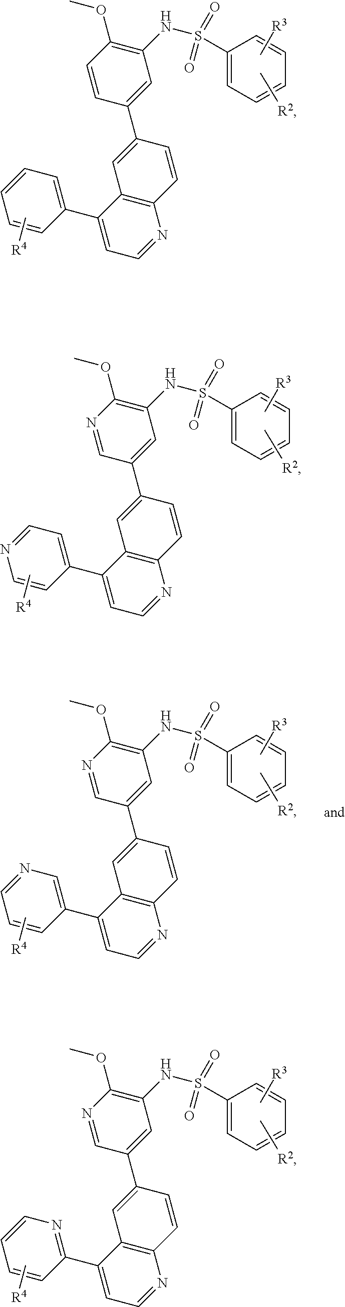

or a pharmaceutically acceptable salt thereof.

Examples of compounds of the formula (II) also include compounds of the formulae:

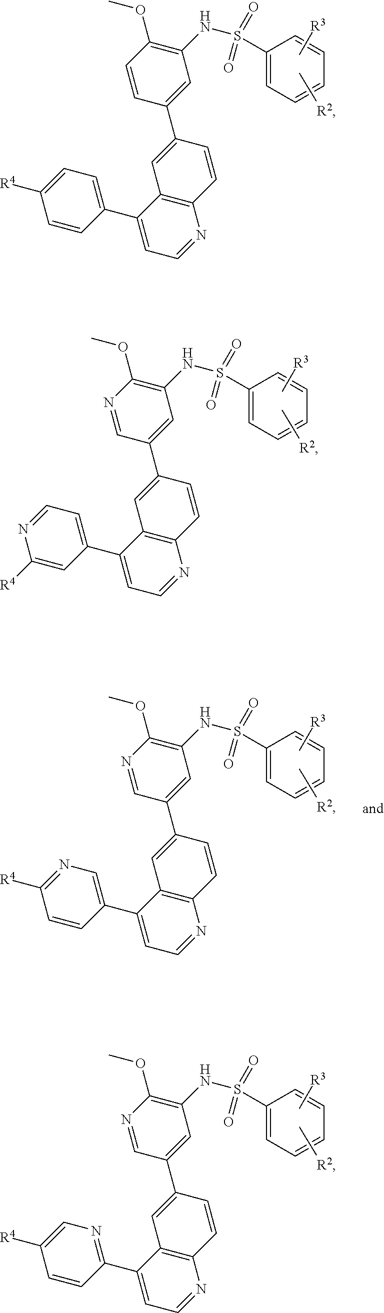

or a pharmaceutically acceptable salt thereof.

In the compounds of formula (II), L can be a hydrolyzable linker. Or L can be an optionally substituted heteroalkyl. For example, the substituted heteroalkyl can be substituted with at least one substituent selected from the group consisting of alkyl, hydroxyl, acyl, polyethylene glycol (PEG), carboxylate, and halo. In other examples, L can be a substituted heteroalkyl with at least one disulfide bond in the backbone thereof.

In still other examples, L can be a peptide or a peptidoglycan with at least one disulfide bond in the backbone thereof. For example, L can have the formula:

—CO—(CH₂)₂—CONH—CH(COOH)—CH₂—CR⁶R⁷—S—S—CH₂—O—CO—

wherein R⁶ and R⁷ are each, independently, H, alkyl, or heteroalkyl (e.g., polyethylene glycol (PEG)).

In yet other examples, L is a group or comprises a group of the formula:

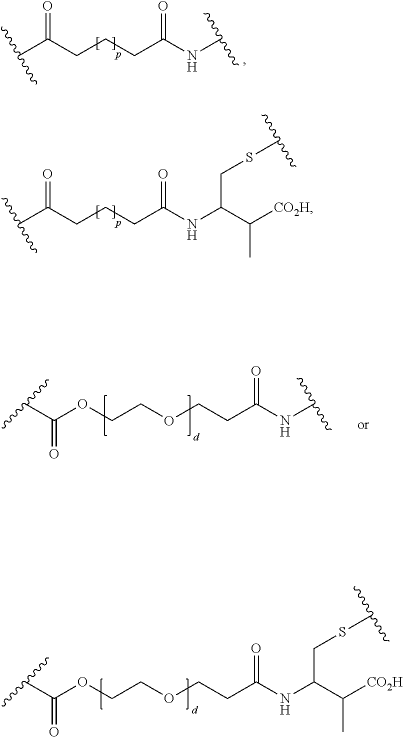

wherein p is an integer from 0 to 10 (e.g., 1 to 5, 2 to 4, 3 to 5, or 1 to 3) and d is an integer from 1 to 40 (e.g., 1 to 32, 2 to 10, 1 to 5, 8 to 20, or 1 to 8).

The FAP ligand corresponding to D in compounds of the formula (II) is a group or can comprise a group of the formulae (III)-(V):

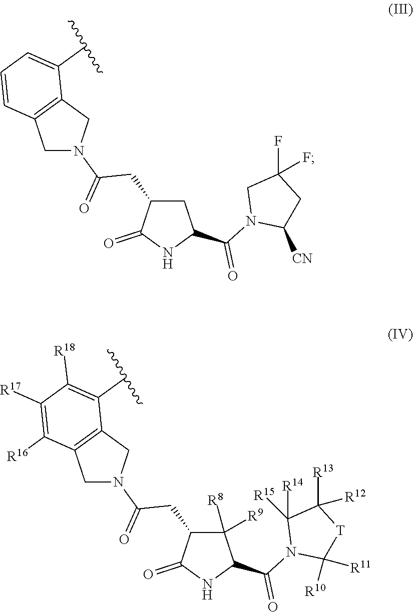

wherein,
T is CH₂, NH, O or S;
R¹⁰ and R¹¹ are each, independently, —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F or 5-tetrazolyl;
R¹² and R¹³ are each, independently, —H, —OH, F, Cl, Br, I, —C₁₋₆alkyl, —O—C₁₋₆alkyl, or —S—C₁₋₆alkyl;
R⁸, R⁹, R¹⁴, and R¹⁵ are each, independently, H, alkyl or halo; and
R16-R¹⁸ are each, independently, H, —C₁₋₆alkyl, —O—C₁₋₆alkyl, —S—C₁₋₆ alkyl, F, Cl, Br, and I; or

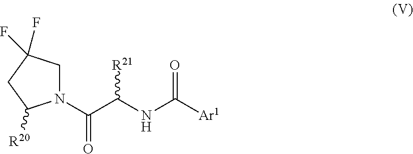

wherein,
R²⁰ is —H, —CN, —B(OH)₂, —C(O)alkyl, —C(O)aryl, —C═C—C(O)aryl, —C═C—S(O)₂aryl, —CO₂H, —SO₃H, —SO₂NH₂, —PO₃H₂, or 5-tetrazolyl;
R²¹ is H or CH₃; and
Ar¹ is substituted phenyl, pyridyl, chloropyridyl, or quinolinyl.

Examples of compounds of the formula (II), which incorporate linkers (L) and FAP ligands (D), include compounds of the formulae:

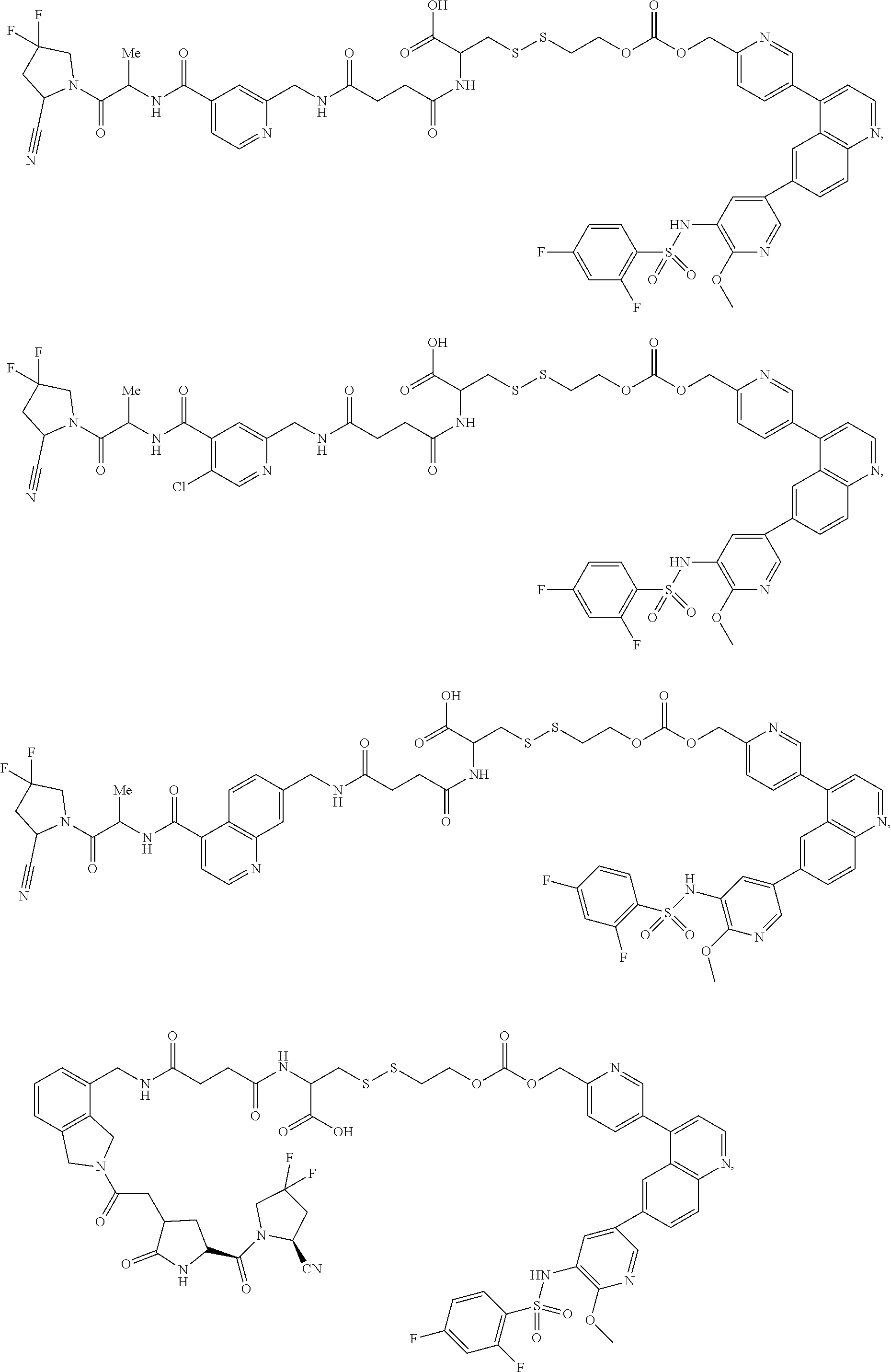

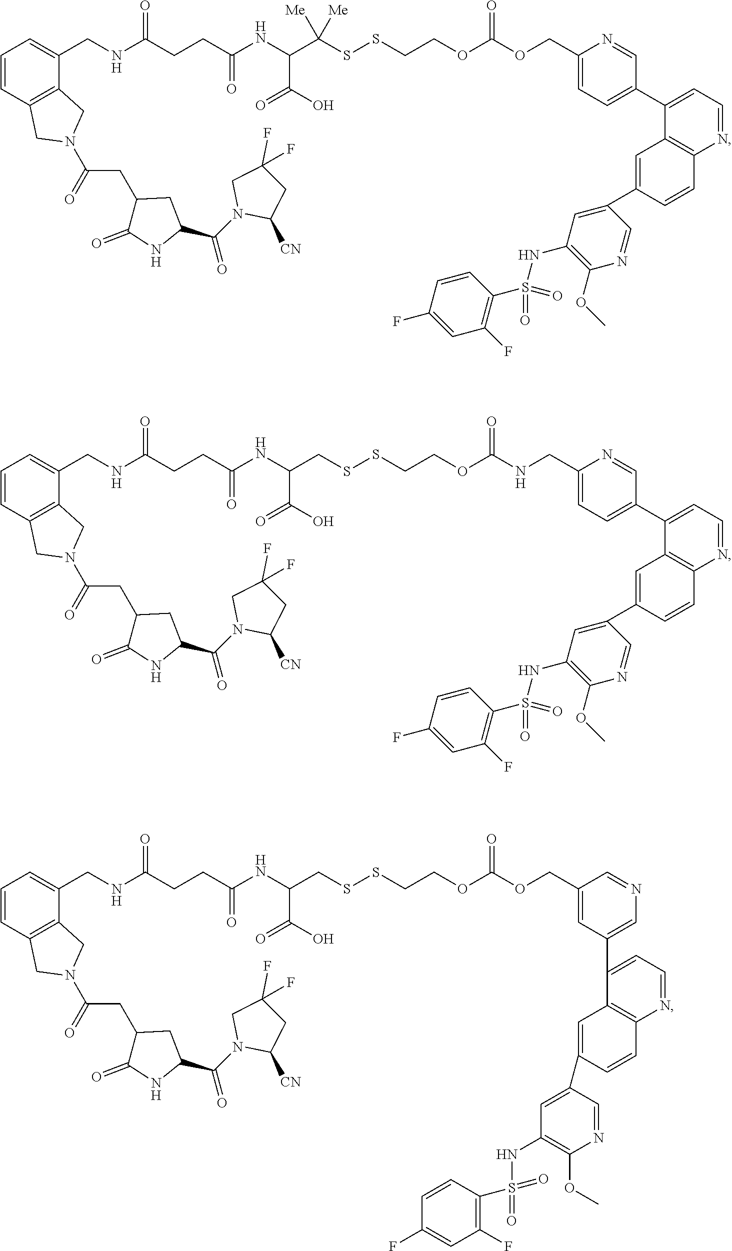

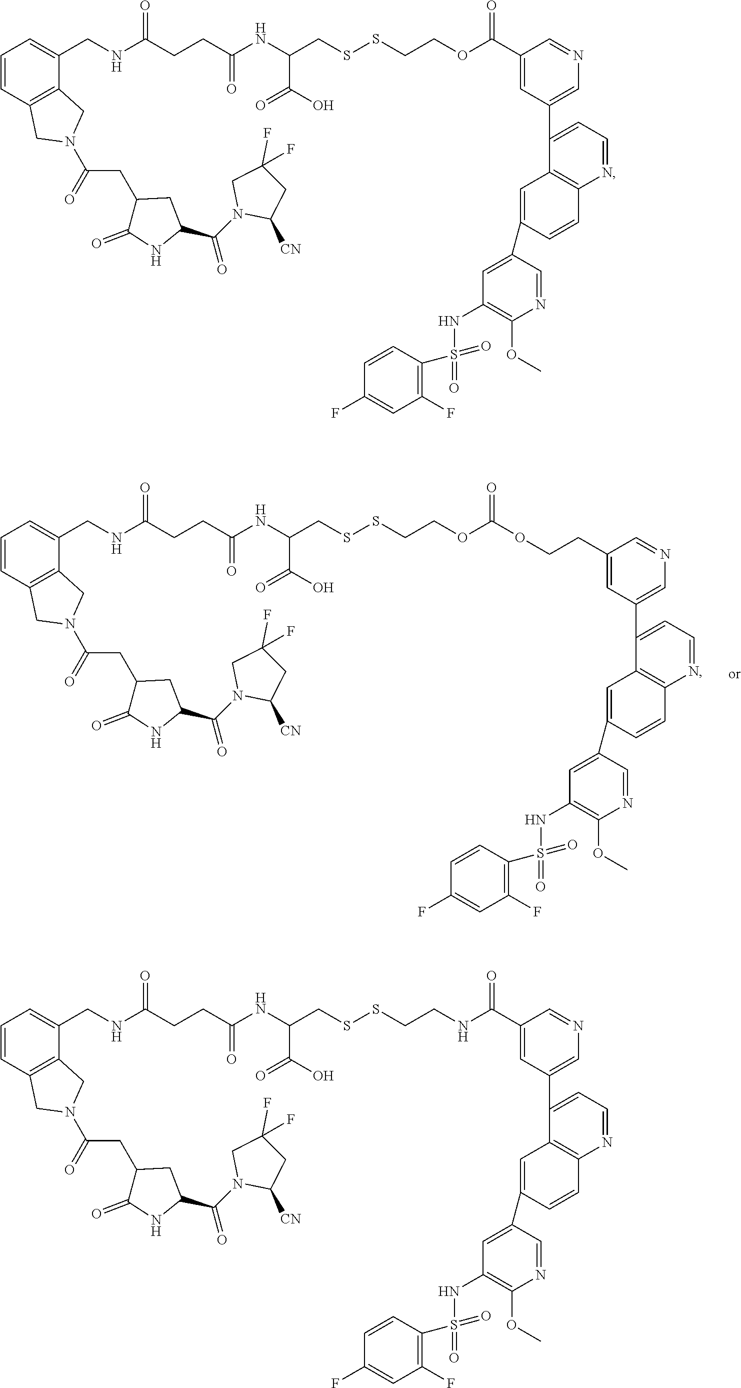

or a pharmaceutically acceptable salt thereof.

### Pharmaceutical Compositions, Routes of Administration, and Dosing

Also provided are pharmaceutical compositions comprising one or more compounds described herein (e.g., a compound of the formula (II)) and one or more pharmaceutically acceptable carriers, diluents, excipients or combinations thereof. A “pharmaceutical composition” refers to a chemical or biological composition suitable for administration to a subject (e.g., mammal). Such compositions can be specifically formulated for administration via one or more of a number of routes including, but not limited to, buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it can provide chemical anther biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, and isotonic and absorption delaying agents that are physiologically compatible. The carrier can be suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well-known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol. (e.g., glycerol, propylene glycol, and liquid polyethylene glycol), and suitable mixtures thereof. The proper fluidity can be maintained, for example, by the use of a coating, such as lecithin, by the maintenance of the required particle size in the case of dispersion, and by the use of surfactants.

In some cases isotonic agents can be included in the pharmaceutical compositions. Examples include sugars, polyalcohols, such as mannitol, sorbitol, and sodium chloride. Prolonged absorption of the injectable compositions can be brought about by including in the composition an agent which delays absorption such as, for example, monostearate salts and gelatin. Moreover, the compounds can be formulated in a time- release formulation, for example, in a composition that includes a slow-release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated. The pharmaceutical compositions can be orally administered as a capsule (hard or soft), tablet (film coated, enteric coated or uncoated), powder, granules (coated or uncoated), or liquid (solution or suspension). The formulations can be conveniently prepared by any of the methods well-known in the art. The pharmaceutical compositions can include one or more suitable production aids or excipients including fillers, hinders, disintegrants, lubricants, diluents, flow agents, buffering agents, moistening agents, preservatives, colorants, sweeteners, flavors, and pharmaceutically compatible carriers.

The compounds can be administered by a variety of dosage forms as known in the art. Any biologically acceptable dosage form known to persons of ordinary skill in the art, and combinations thereof, are contemplated. Examples of such dosage forms include, without limitation, chewable tablets, quick-dissolve tablets, effervescent tablets, reconstitutable powders, elixirs, liquids, solutions, suspensions, emulsions, tablets, multi-layer tablets, hi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds, which can be included by admixture are, for example, medically inert ingredients (e.g., solid and liquid diluent), such as lactose, dextrose saccharose, cellulose, starch or calcium phosphate for tablets or capsules, olive oil or ethyl oleate for soft capsules and water or vegetable oil for suspensions or emulsions; lubricating agents, such as silica, talc, stearic acid, magnesium or calcium stearate and/or polyethylene glycols; gelling agents, such as colloidal clays; thickening agents, such as gum tragacanth or sodium alginate; binding agents, such as starches, arabic gums, gelatin, methylcellulose, carboxymethylcellulose or polyvinylpyrrolidone; disintegrating agents, such as starch, alginic acid, alginates or sodium starch glycolate; effervescing mixtures; dyestuff sweeteners; wetting agents, such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition can vary according to factors such as the disease state, age, gender, weight, patient history, risk factors, predisposition to disease, administration route, pre-existing treatment regime (e.g., possible interactions with other medications), and weight of the individual. Dosage regimens can be adjusted to provide the optimum therapeutic response. For example, a single bolus can be administered, several divided doses can be administered over time, or the dose can be proportionally reduced or increased as indicated by the exigencies of therapeutic situation.

“Dosage unit form,” as used herein, refers to physically discrete units, suited as unitary dosages, for the mammalian subjects to be treated; each unit containing a predetermined quantity of active compound calculated to produce the desired therapeutic effect in association with the required pharmaceutical carrier. The specification for the dosage unit forms are dictated by, and directly dependent on, the unique characteristics of the active compound, the particular therapeutic effect to be achieved, and the limitations inherent in the art of compounding such an active compound for the treatment of sensitivity in individuals.

The dosage can be administered once, twice, or thrice a day, although more frequent dosing intervals are possible. The dosage can be administered every day, every 2 days, every 3 days, every 4 days, every 5 days, every 6 days, and/or every 7 days (once a week). The dosage can be administered daily for up to and including 30 days, preferably between 7-10 days. The dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition can effect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, i.e., the composition is applied directly where its action is desired. Administration can be enteral when the desired effect is systemic (non-local), i.e., the composition is given via the digestive tract. Administration can be parenteral, when the desired effect is systemic, i.e., the composition is given by other routes than the digestive tract.

Compositions comprising a therapeutically effective amount of one or more compounds described herein (e.g., a compound of the formula (II)) are also contemplated. The compositions are useful in a method for treating fibrosis (e.g., idiopathic pulmonary fibrosis), the method comprising administering a therapeutically effective amount of one or more compounds described herein to a patient in need thereof. Also contemplated herein is one or more compounds described herein for use as a medicament for treating a patient in need of relief from fibrosis (e.g., idiopathic pulmonary fibrosis).

The term “therapeutically effective amount” as used herein, refers to that amount of one or more compounds described herein (e.g., a compound of the formula (II)) that elicits a biological or medicinal response sought by a researcher, a veterinarian, a medical doctor or other clinician, which includes alleviation of the symptoms of the disease or disorder being treated. The therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well-known to the researcher, veterinarian, medical doctor or other clinician. It is also appreciated that the therapeutically effective amount can be selected with reference to any toxicity, or other undesirable side effect, that might occur during administration of one or more of the compounds.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference for its teachings regarding same.

### Method of Treatment

This disclosure further provides a method of treating fibrosis in a subject in need thereof.

The methods can be used for both human clinical medicine and veterinary applications. Thus, a “subject” can be administered a compound in accordance with the present teachings, and can be a human “patient”) or, in the case of veterinary applications, can be a laboratory, agricultural, domestic, or wild animal. The subject can be a human patient, a laboratory animal, such as a rodent (e.g., mice, rats, hamsters, etc.), a rabbit, a monkey, or a chimpanzee, a domestic animal, such as a dog, a cat, or a rabbit, an agricultural animal, such as a cow, a horse, a pig, a sheep, or a goat, and a wild animal in captivity, such as a bear, a panda, a lion, a tiger, a leopard, an elephant, a zebra, a giraffe, a gorilla, a dolphin, or a whale.

Any of the methods disclosed herein comprises the step of providing to the subject a therapeutically effective amount of compound of formula (II) for example.

The entire contents of each and every patent publication, non-patent publication, and reference text cited herein are hereby incorporated by reference, except that in the event of any inconsistent disclosure or definition from the present specification, the disclosure or definition herein shall be deemed to prevail.

### Certain Definitions

The singular forms “a”, “an” and “the” include plural referents unless the content clearly dictates otherwise. Thus, for example, where a compound or composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

If a chemical group combines several other chemical groups defined herein, then each part of the combination is assumed to be defined as when it is separate, with allowances made to create valences for allowing attachment of the other groups. For example, “alkoxycycloalkylenecarbonyl” radical would be understood to be an alkoxy as defined herein bonded to a cycloalkylene as defined herein, and the cycloalkylene is, in turn, bonded to a carbonyl group, which is not defined herein but is generally understood by organic chemists, with an open valence on the carbonyl.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substituted” or “substituent” as used herein refers to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH2)₀₋₂P(O)(OR)₂, C(O)R, C(O)C(O)R, C(O)CH2C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH2)₀₋₂N(R)N(R)2, N(R)N(R)C(O)R, N(R)N(R)C(O)OR, N(R)N(R)CON(R)2, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched alkyl groups and cycloalkyl groups having from 1 to 40 carbon atoms (C1-C40), 1 to about 20 carbon atoms (C1-C20), 1 to 12 carbons (C1-C12), 1 to 8 carbon atoms (C1-C8), or from 1 to 6 carbon atoms (C1-C6). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. The cycloalkyl group can have 3 to about 8-12 ring members, or the number of ring carbon atoms range from 3 to 4, 5, 6, or 7. Cycloalkyl groups can have 3 to 6 carbon atoms (C3-C6). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl. aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, acryloyl groups, and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. Aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-. 3-, 4-, 5-, or 6-substituted phenyl or 2-8 substituted naphthyl groups, which can be substituted with carbon or non-carbon groups, such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” as used herein refers to substituted or unsubstituted aromatic and non-aromatic ring compounds containing 3 or more ring members, of which, one or more (e.g., 1, 2 or 3) is a heteroatom such as, but not limited to, N, O, and S. Thus, a heterocyclyl can be a cycloheteroalkyl, or a heteroaryl, or if polycyclic, any combination thereof. Heterocyclyl groups include 3 to about 20 ring members, whereas other such groups have 3 to about 15 ring members. Heterocyclyl groups include heterocyclyl groups that include 3 to 8 carbon atoms (C₃-C₈), 3 to 6 carbon atoms (C₃-C₆), 3 to 5 carbon atoms (C₃-C₅) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise. a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups. Examples of indolinonyl groups include groups having the general formula:

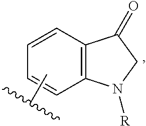

wherein R is as defined herein.
Examples of isoindolinonyl groups include groups having the general formula:

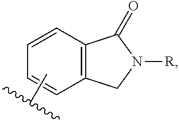

wherein R is as defined herein.
Examples of benzoxazolinyl groups include groups having the general formula:

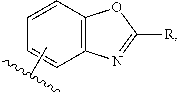

wherein R is as defined herein.
Examples of benzthiazolinyl groups include groups having the general formula:

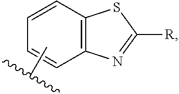

wherein R is as defined herein.
The group R in benzoxazolinyl and benzthiazolinyl groups can be an N(R)₂ group. Each R can be hydrogen or alkyl, wherein the alkyl group is substituted or unsubstituted. The alkyl group can be substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, teat-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” refers to primary, secondary, and tertiary amines having, e.g., the formula N(group)₃ wherein each group can independently be H or non-H, such as alkyl, aryl, and the like. Amines include, but are not limited to, R—NH₂, for example, alkylamines, arylamines, alkylarylamines; R₂NH, wherein R is defined herein, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

An example of a “alkylamino” is —NFL-alkyl and —N(alkyl)₂.

The terms “halo,” “halogen,” or “halide” group, by themselves or as part of another substituent, mean, unless otherwise stated, a fluorine, chlorine, bromine, or iodine atom.

The terms “salts” and “pharmaceutically acceptable salts” refer to derivatives of the disclosed compounds, wherein the parent compound is modified by making acid or base salts thereof. Examples of pharmaceutically acceptable salts include, but are not limited to, mineral or organic acid salts of basic groups, such as amines; and alkali or organic salts of acidic groups, such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids, such as hydrochloric, hydrobromic, sulfuric, sulfamic, phosphoric, and nitric; and the salts prepared from organic acids, such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

In the methods, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Unless defined otherwise, all technical and scientific terms used herein have the same meaning as commonly understood to one of ordinary skill in the art to which this disclosure belongs. Although any methods, devices, and materials similar or equivalent to those described herein can be used in the practice or testing of the present teachings, the preferred methods, devices and materials are now described.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It will be understood by one of ordinary skill in the relevant arts that other suitable modifications and adaptations to the compositions and methods described herein are readily apparent from the description of the disclosure contained herein in view of information known to the ordinarily skilled artisan, and can be made without departing from the scope of the disclosure. Having now described the present disclosure in detail, the same will be more clearly understood by reference to the following examples, which are included herewith for purposes of illustration only and are not intended to be limiting of the disclosure.

### NUMBERED EMBODIMENTS

Embodiment 1 relates to compound of the formula (I):

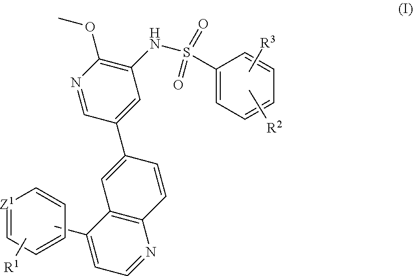

or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R¹ is hydroxyalkyl, aminoalkyl, —S(O)ₓalkyl (wherein x is 0, 1 or 2), carboxyl, carboxylalkyl, thiocarboxyl, thiocarboxylalkyl, amido or amidoalkyl;
R² and R³ are each, independently, H, halo, hydroxy, alkyl, alkoxy, aryl, amino, acyl or C(O)R^{b}, wherein R^{b} is alkyl, aryl, OH or alkoxy.

Embodiment 2 relates to a compound of Embodiment 1, wherein the compound is a compound of the formula:

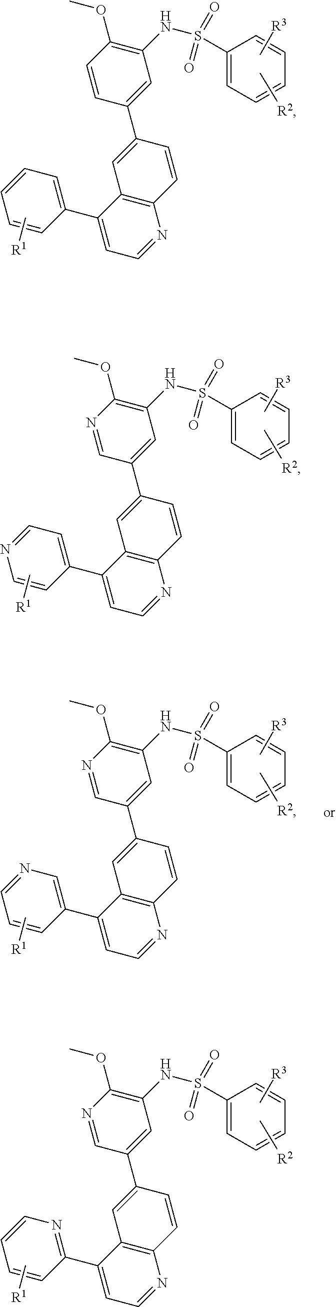

or a pharmaceutically acceptable salt thereof.

Embodiment 3 relates to a compound of Embodiment 1, wherein the compound is a compound of the formula:

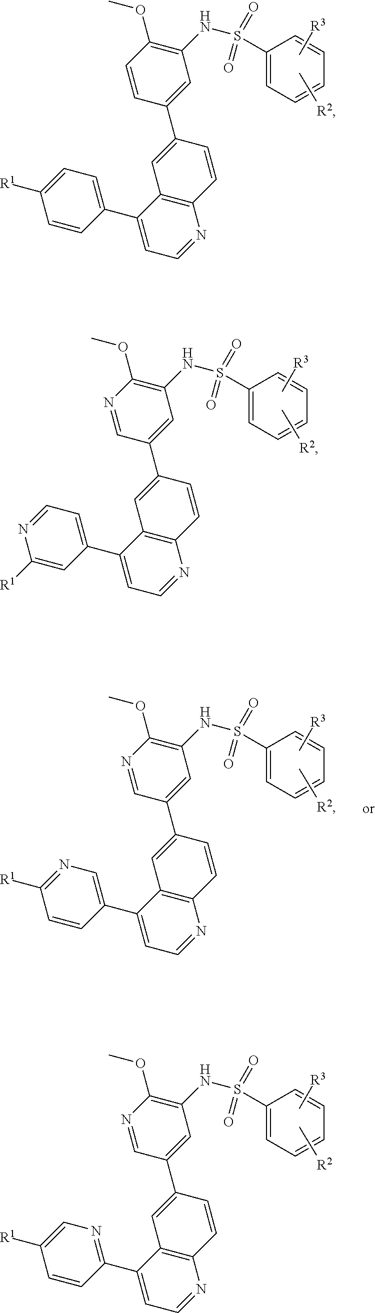

or a pharmaceutically acceptable salt thereof.

Embodiment 4 relates to a compound of any one of Embodiments 1-3, wherein R¹ is a group of the formula R^{c}O-alkyl-, wherein R^{c} is H or a hydroxyl protecting group; (R^{d})₂N-alkyl-, wherein R^{d} is H or an amine protecting group; R^{e}S(O)ₓ-alkyl-; R^{e}O(O)C—; R^{e}O(O)C-alkyl-; R^{e}S(O)C—; R^{e}S(O)C-alkyl-; (R^{e})₂N(O)C—; or (R^{e})₂N(O)C-alkyl-; wherein R^{e} is H or alkyl, and x is 0, 1, or 2.

Embodiment 5 relates to a compound of any one of Embodiments 1-4, wherein R¹ is a group of the formula R^{c}O(CH₂)ₙ—, wherein R^{e} is H or a hydroxyl protecting group; (R^{d})₂N(CH₂)ₙ— wherein R^{d} is H or an amine protecting group; R^{e}S(O)ₓ(CH2)ₙ—, wherein R^{e} is H or alkyl, and x is 0, 1, or 2; R^{e}O(O)C(CH₂)ₙ—, wherein R^{e} is H or alkyl; R^{e}S(O)C—; R^{e}S(O)C(CH₂)ₙ—, wherein R^{e} is H or alkyl; or (R^{e})₂N(O)C(CH₂)ₙ—, wherein R^{e} is H or alkyl; wherein n is an integer from 1 to 20.

Embodiment 6 relates to a compound of any one of Embodiments 1-5, wherein the compound is a compound of the formula:

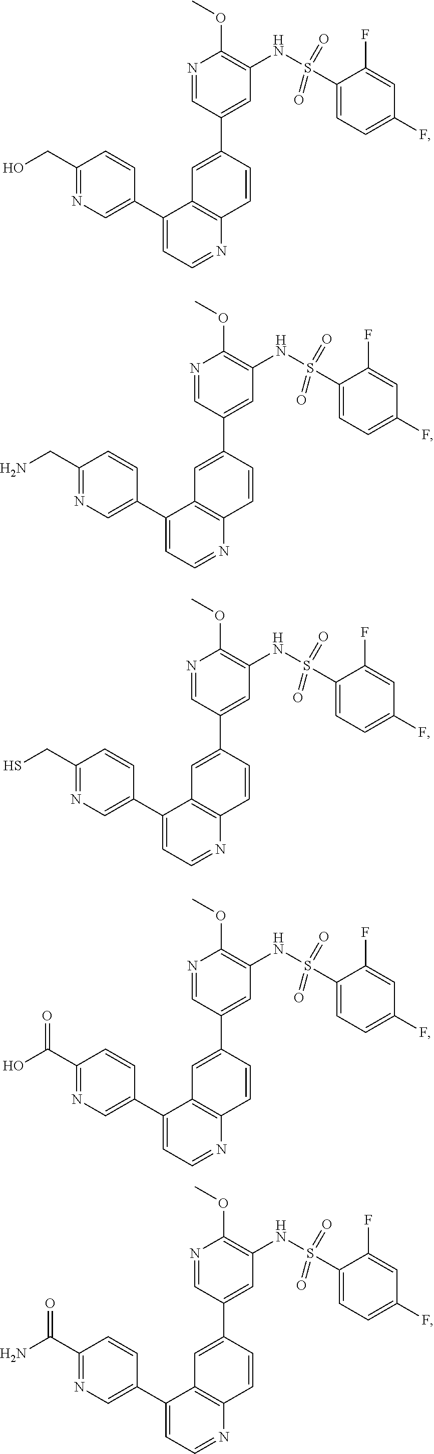

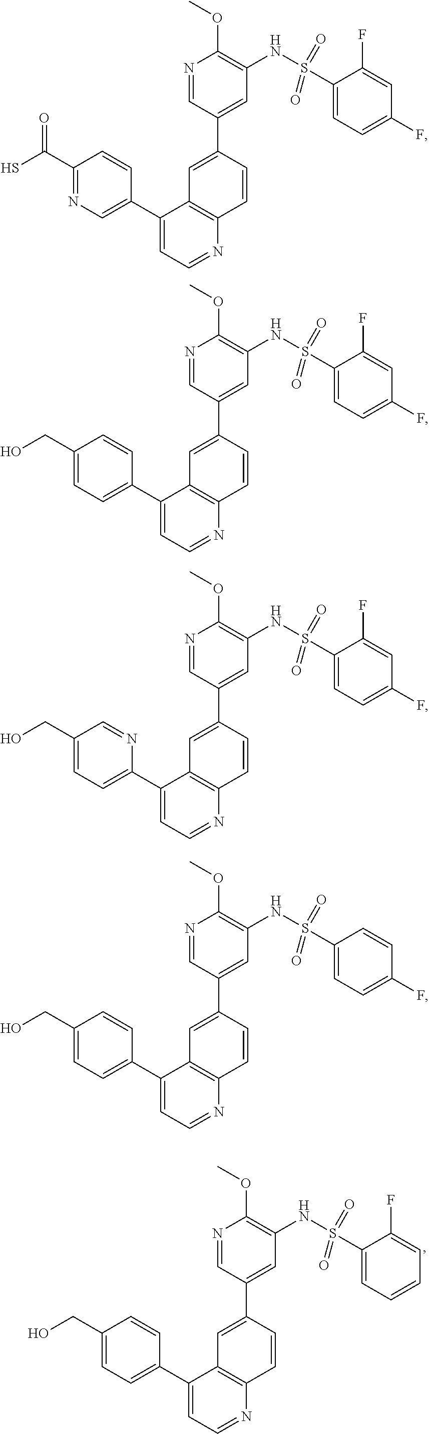

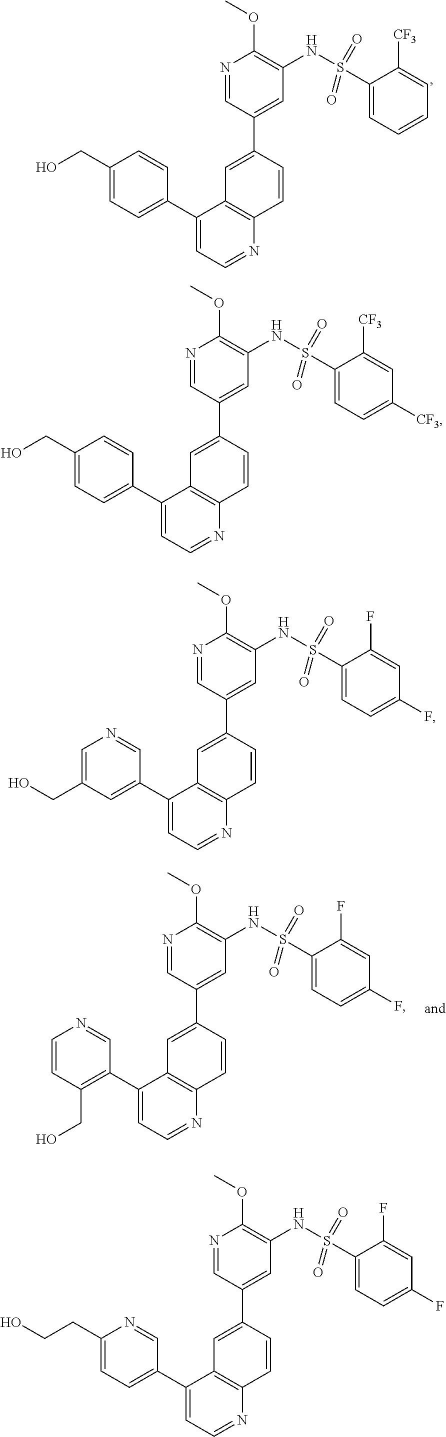

or a pharmaceutically acceptable salt thereof.

Embodiment 7 relates to a compound of the formula (II):

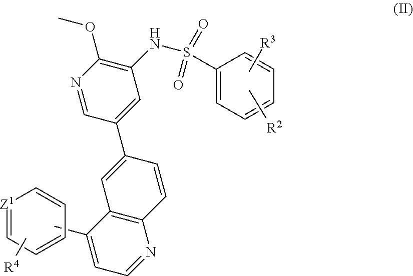

or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R⁴ is a group of the formula D-L-O-alkyl-, D-L-N(R^{e})-alkyl-, D-L-S(O)ₓalkyl, D-L-C(O)— or D-L-C(O)-alkyl, wherein L is a linker, and D is a FAP ligand; and
R² and R³ are each, independently, H, halo, hydroxy, alkyl., alkoxy, aryl, amino, acyl or C(O)R^{b}, wherein R^{b} is alkyl, aryl, OH or alkoxy.

Embodiment 8 relates to a compound of Embodiment 7, wherein the compound is a compound of the formula:

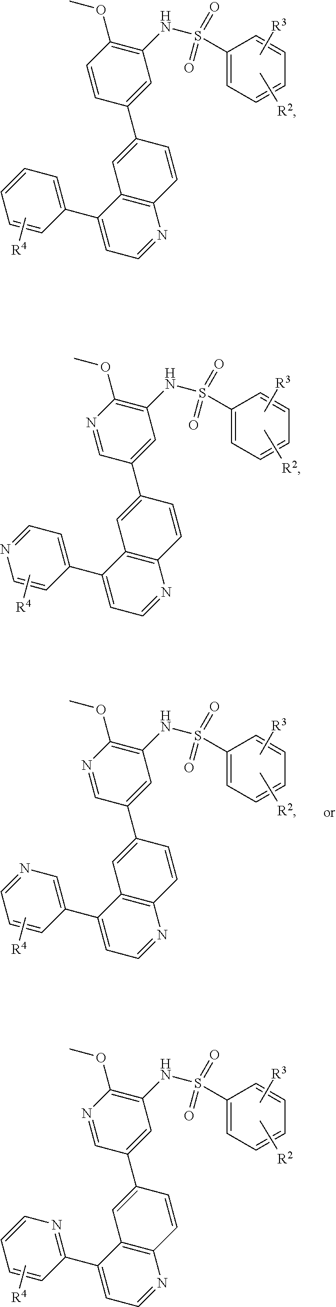

or a pharmaceutically acceptable salt thereof.

Embodiment 9 relates to a compound of Embodiment 7, wherein the compound is a compound of the formula:

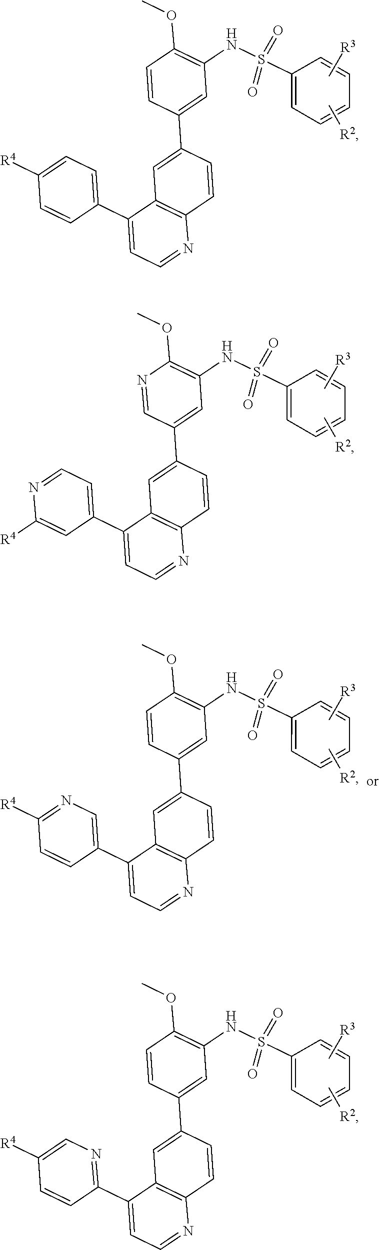

or a pharmaceutically acceptable salt thereof.

Embodiment 10 relates to a compound of any one of Embodiments 7-9, wherein L is a hydrolyzable linker.

Embodiment 11 relates to a compound of any one of Embodiments 7-9, wherein L is an optionally substituted heteroalkyl.

Embodiment 12 relates to a compound of Embodiment 11, wherein the substituted heteroalkyl is substituted with at least one substituent selected from the group consisting of alkyl, hydroxyl, acyl, polyethylene glycol (PEG), carboxylate, and halo.

Embodiment 13 relates to a compound of any one of Embodiments 7-9, wherein L is a substituted heteroalkyl with at least one disulfide bond in the backbone thereof.

Embodiment 14 relates to a compound of any one of Embodiments 7-9, wherein L is a peptide or a peptidoglycan with at least one disulfide bond in the backbone thereof.

Embodiment 15 relates to a compound of any one of Embodiments 7-9, wherein L has the formula:

—CO—(CH₂)₂—CONH—CH(COOH)—CH₂—CR⁶R⁷—S—S—CH₂—O—CO—,

wherein R⁶ and R⁷ are each, independently, H, alkyl, or heteroalkyl.

Embodiment 16 relates to a compound of any one of Embodiments 7-9, wherein L is a group or comprises a group of the formula:

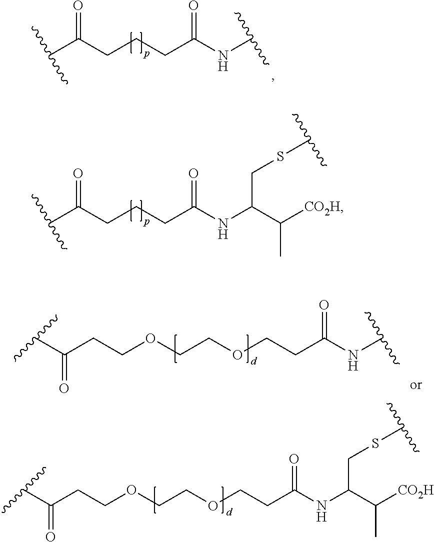

wherein p is an integer from 0 to 10; and d is an integer from 1 to 40.

Embodiment 17 relates to a compound of any one of Embodiments 7-16, wherein D is a group or comprise a group of the formula (III):

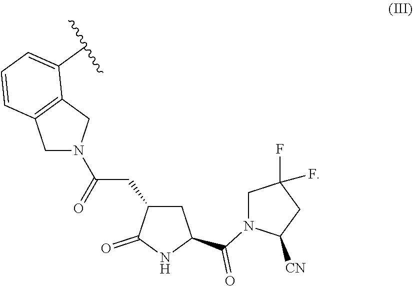 [SMILES: *c1cccc2c1CN(C(=O)C[C@@H]1C[C@@H](C(=O)N3CC(F)(F)C[C@H]3C#N)NC1=O)C2]

Embodiment 18 relates to a compound of any one of Embodiments 7-16, wherein D is a group or comprise a group of the formula (IV):

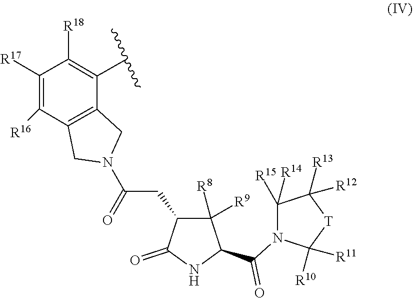

wherein,
T is CH₂, NH, O or S;
R¹⁰ and R¹¹ are each, independently, —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F or 5-tetrazolyl;
R¹² and R¹³ are each, independently, —H, —OH, F, Cl, Br, I, —C₁₋₆alkyl, —O—C₁₋₆alkyl, or —S—C₁₋₆alkyl;
R⁸, R⁹, R¹⁴, and R¹⁵ are each, independently, alkyl or halo; and
R¹⁶-R¹⁸ are each, independently, H, —C₁₋₆alkyl, —O—C₁₋₆alkyl, —S—C₁₋₆ alkyl, F, Cl, Br, or I.

Embodiment 19 relates to a compound of any one of Embodiments 7-16, wherein D is a group or comprise a group of the formula (IV):

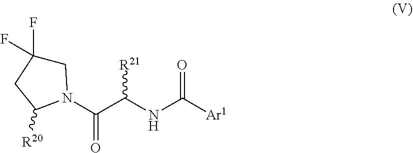

wherein,
R²⁰ is —H, —CN, —B(OH)₂, —C(O)alkyl, —C(O)aryl, —C═C—C(O)aryl, —C═C—S(O)₂aryl, —CO₂H, —SO₃H, —SO₂NH₂, —PO₃H₂, or 5-tetrazolyl;
R²¹ is H or CH₃, and
Ar¹ is substituted phenyl, pyridyl, chloropyridyl, or quinolinyl.

Embodiment 20 relates to a compound of any one of Embodiments 7-19, wherein the compound of the formula (II) is a compound of the formula:

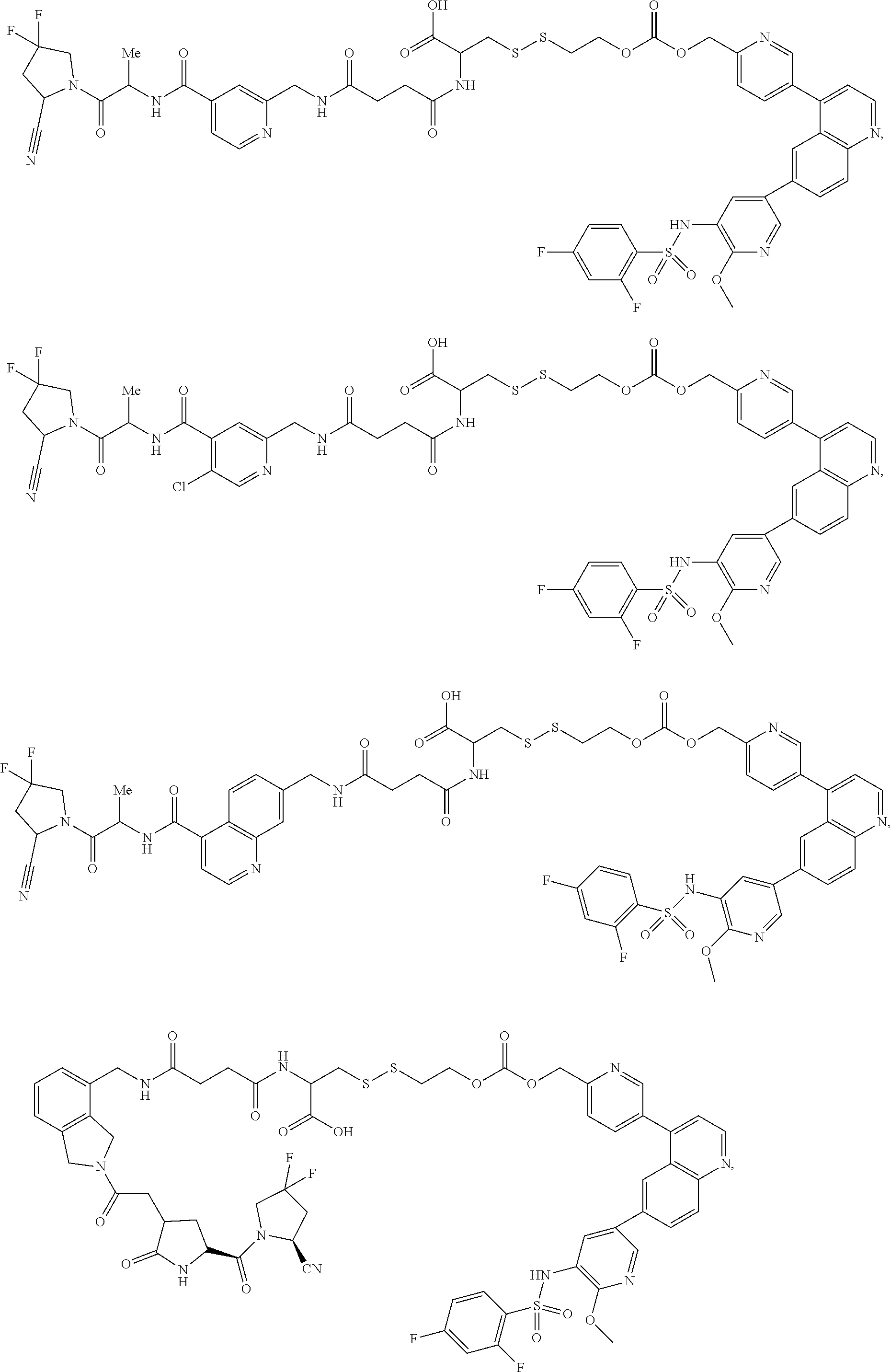

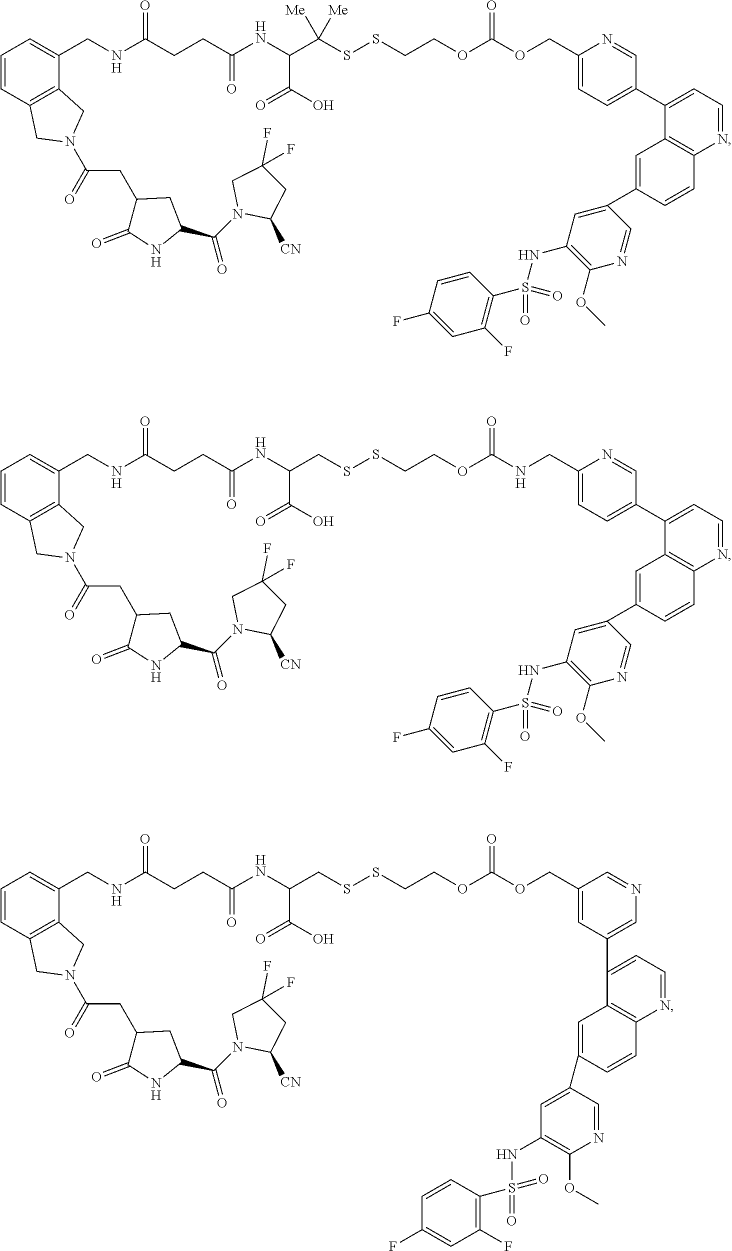

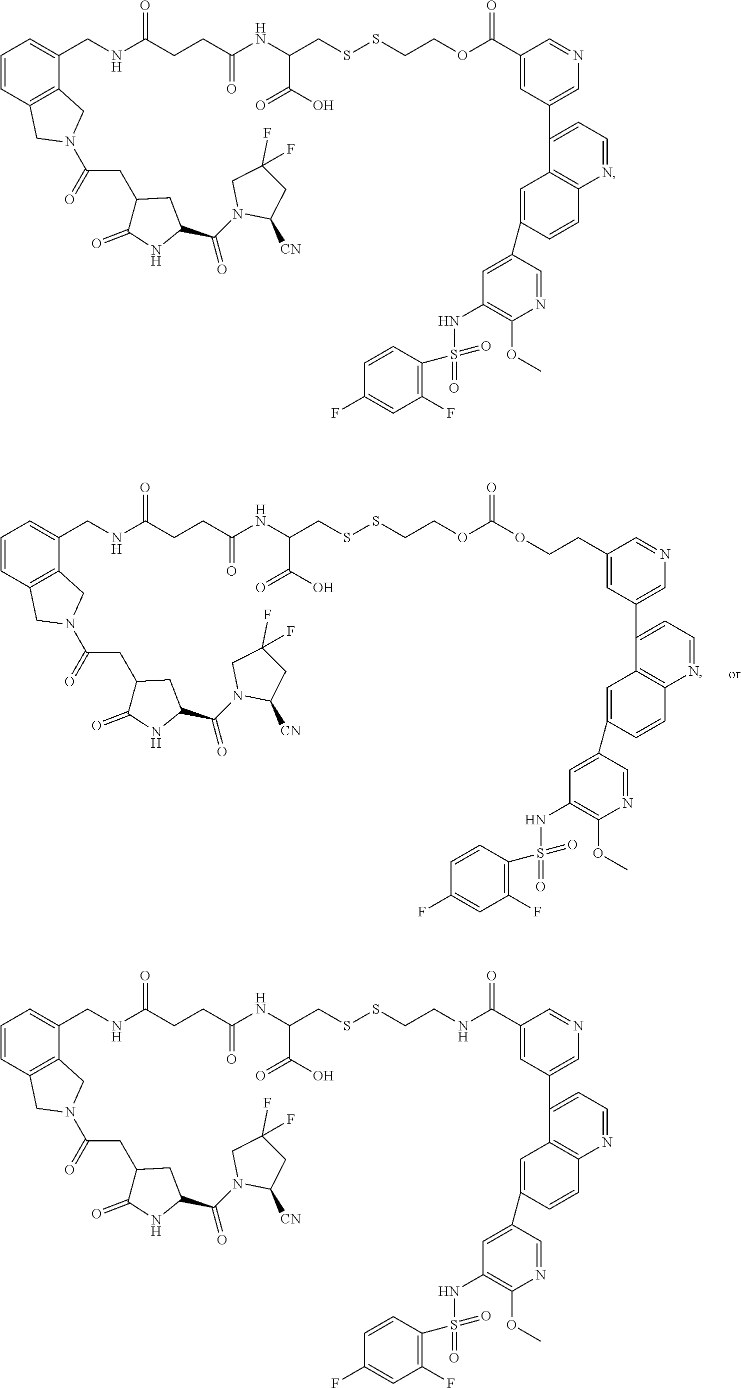

or a pharmaceutically acceptable salt thereof.

Embodiment 21 relates a pharmaceutical composition comprising a therapeutically effective amount of one or more compounds of any one of Embodiments 7-20 and at least one pharmaceutically acceptable excipient.

Embodiment 22 relates to a method for treating fibrosis, the method comprising administering a therapeutically effective amount of one or more compounds of Embodiments 7-20 or a pharmaceutical composition of Embodiment 21 to a subject in need thereof.

### EXAMPLES

The present invention can be better understood by reference to the following examples, which are offered by way of illustration. The present invention is not limited to the examples given herein.

### Illustrative Synthetic Procedures

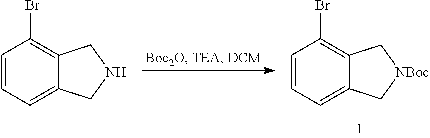 [SMILES: Brc1cccc2c1CNC2 ; CC(C)(C)OC(=O)N1Cc2cccc(Br)c2C1]

Step 1 4-bromoisoindoline (1.97 g, 10 mmol, 1.0 eq) was dissolved in DCM (10 mL). Then Boc₂O (10.9 g, 50 mmol, 5 eq) was added followed by triethylamine (3.03 g, 30 mmol, 3 eq). The mixture was kept for 8 hrs. Quench the reaction with water (15 mL) and extract the aqueous layer with DCM (10 mL*3). Combined the organic phase and dry with sodium sulfate. Concentrated under reduced pressure. Purified through combi with hexane/ethyl acetate as eluent. Compound 1 was obtained in 2.01 g as white solid.

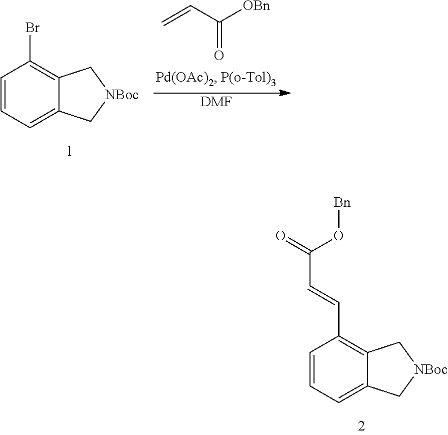

Step 2 under N₂ atmosphere, compound 1 (297 mg, 1 mmol, 1.0 eq) was dissolved in DMF (1 mL). Then benzyl acrylate (486 mg, 3 mmol, 3.0 eq) was added followed by Pd(OAc)2 (22.3 mg, 0.1 mmol, 0.1 eq) as well as P(o-Tol)3 (60.8 mg, 0.2 mmol, 0.2 eq) and DIPEA (387 mg, 3.0 mmol, 3.0 eq). The resulting mixture was heated at 100° C. for 8 hrs. After completion, quench the reaction with water (3 mL). Extract the aqueous layer with ethyl acetate (10 mL×3). Combined the organic phase and dry with sodium sulfate. Concentrated under reduced pressure. Purified through combi with hexane/ethyl acetate as eluent. Compound 2 was obtained in 148 mg as yellowish oil.

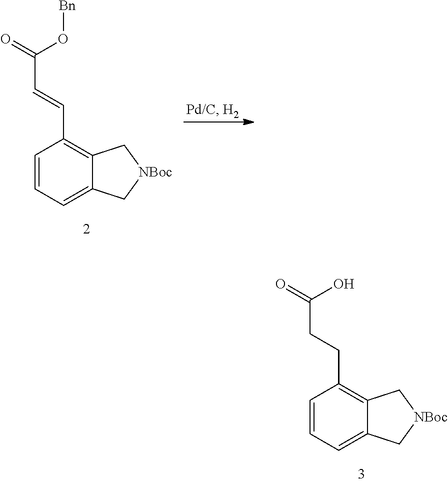 [SMILES: CC(C)(C)OC(=O)N1Cc2cccc(/C=C/C(=O)OCc3ccccc3)c2C1 ; CC(C)(C)OC(=O)N1Cc2cccc(CCC(=O)O)c2C1]

Step 3 under H₂ atmosphere, compound 2 (800 mg, 0.72 mmol) was dissolved in MeOH (20 mL). Then Pd/C (80 mg) was added. The resulting mixture was stirred for 8 hrs. After completion, remove the catalyst through filtration with celite. Concentration under reduced pressure. Compound 3 was obtained as white solid which could be used in the next step without further purification.

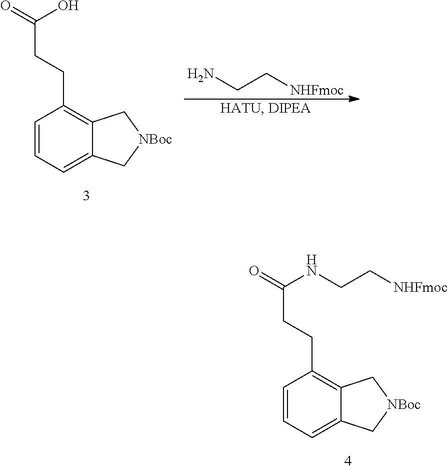

Step 4 under N₂ atmosphere, compound 3 (291 mg, 1.0 mmol, 1.0 eq) was dissolved in DMF (5 mL). Then HATU (456 mg, 1.2 mmol, 1.2 eq) was added followed by DIPEA (258 mg, 2 mmol, 2.0 eq). The mixture was kept for 10 min. Finally, (9H-fluoren-9-yl)methyl (2-aminoethyl)carbamate hydrochloride salt (350.9 mg, 1.1 mmol, 1.1 eq) was added. The reaction was monitored LC-MS until the acid was completely consumed. Diluted with ethyl acetate (2 mL) and wash with H₂O (2 mL*3). Dry with sodium sulfate. Concentrated under reduced pressure. Purified through combi with hexane/ethyl acetate as eluent. Compound 4 was obtained in 376.8 mg as white solid.

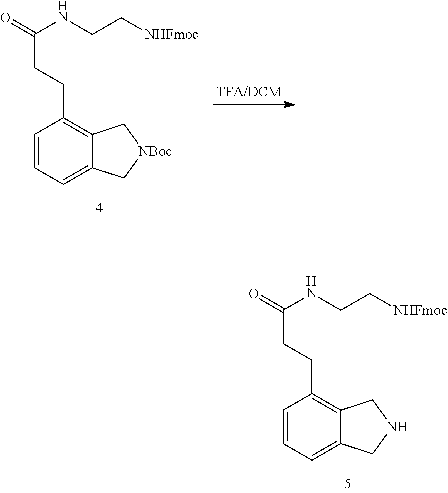

Step 5 under N₂ atmosphere, compound 4 (200 mg, mmol) was dissolved in TFA/DCM (0.2 mL/0.2 mL). The mixture was stirred for 1 h. Remove the solvent under reduced pressure with rotary evaporator. Compound 5 was obtained which could be used in the next step without further purification.

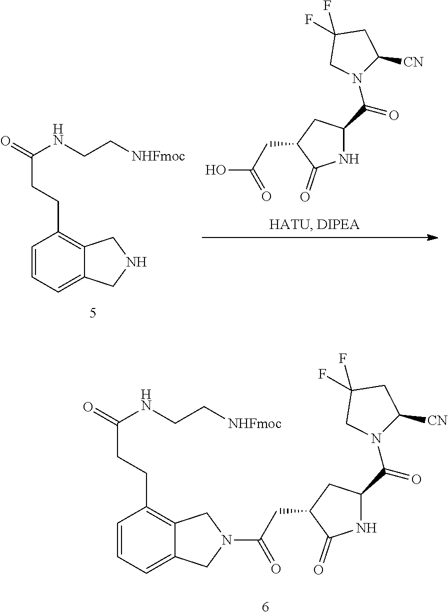

Step 6 under N₂ atmosphere, carboxylic acid (30.1 mg, 0.1 mmol, 1.0 eq) was dissolved in DMF (1 mL). Then HATU (45.6 mg, 0.12 mmol, 1.2 eq) was added followed by DIPEA (18.9 mg, 0.15 mmol, 1.5 eq). The mixture was kept for 10 min. Finally, compound 5 (50.05 mg, 0.11 mmol, 1.1 eq) was added. Monitor the reaction with LC-MS till acid was completely consumed. Diluted with EA (2 mL) and wash with H₂O (2 mL*3). Dry with sodium sulfate. Concentrated under reduced pressure. Purified through combi with DCM/MeOH as eluent. Compound 6 was obtained in 30.7 mg as yellow oil.

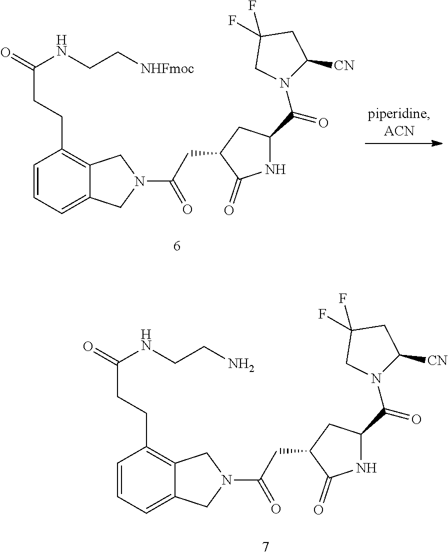

Step 7 under N₂ atmosphere, compound 6 (20 mg, 0.027 mmol) was dissolved in ACN/piperidine (0.2 mL/0.2 mL). The mixture was stirred for 1 h. Remove the solvent under reduced pressure with rotary evaporator. Compound 7 was obtained which could be used in the next step without further purification.

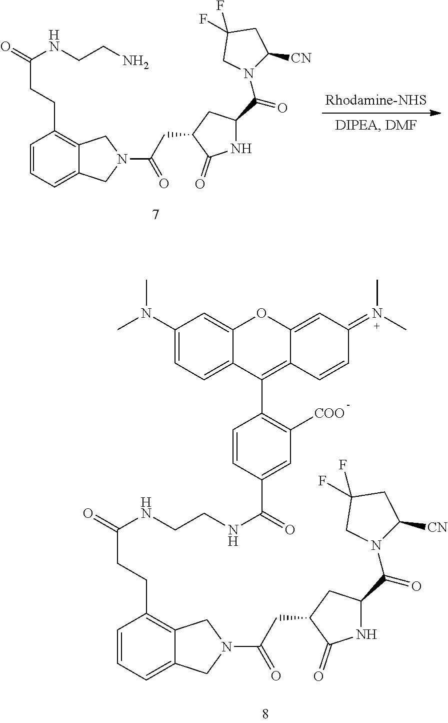

Step 8 under N₂ atmosphere, compound 7 (10.0 mg, 0.019 mmol, 1.0 eq) was dissolved in DMF (1 mL), then Rhodamine-NHS (12.03 mg, 0.0228 mmol, 1.2 eq) was added followed by DIPEA (3.67 mg, 0.0285 mmol,1.5 eq). The mixture was kept for 2 h. After purification, compound 8 was provided in 1.4 mg as pink powder. Purification condition: reverse phase C-18 column, ACN/NH₄HCO₃, pH=7, flow rate 8 mL/min. Rt=25 min. Chemical Formula: C₅₀H₅₀F₂N₈O₈, Exact Mass: 928.4, [M⁺H]⁺ found 929.3.

### Introduction

In order to deliver drugs specifically to activated idiopathic pulmonary fibrosis (IPF) lung myofibroblasts, compounds were designed that comprise two motifs, namely, an IPF-specific receptor and an associated targeting ligand. The compounds described herein comprise both motifs. And such compounds can be exploited for selective delivery of antifibrotic drugs to activated profibrotic fibroblasts.

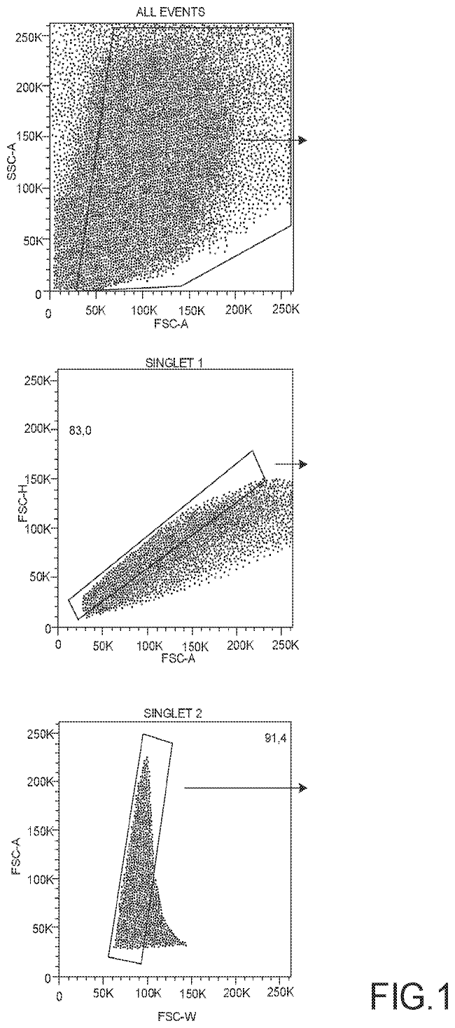

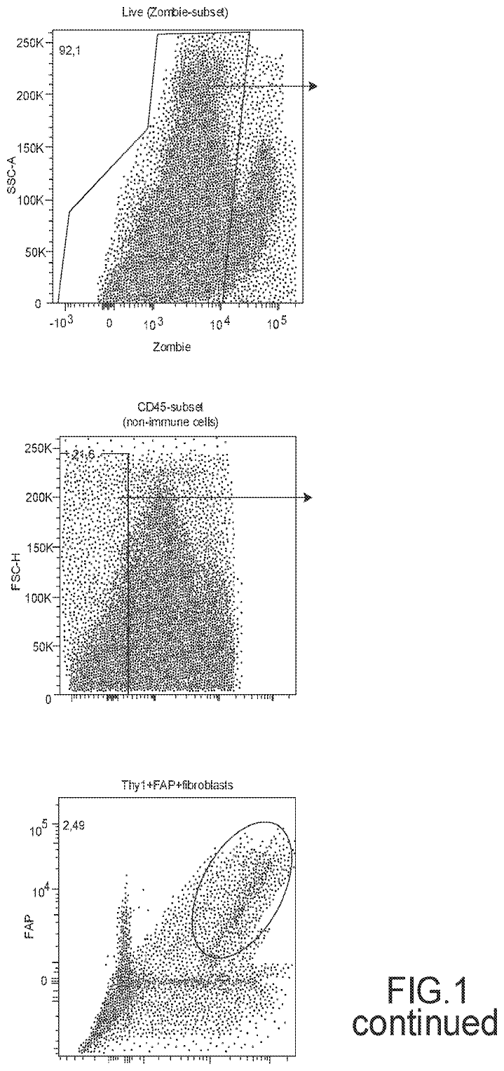

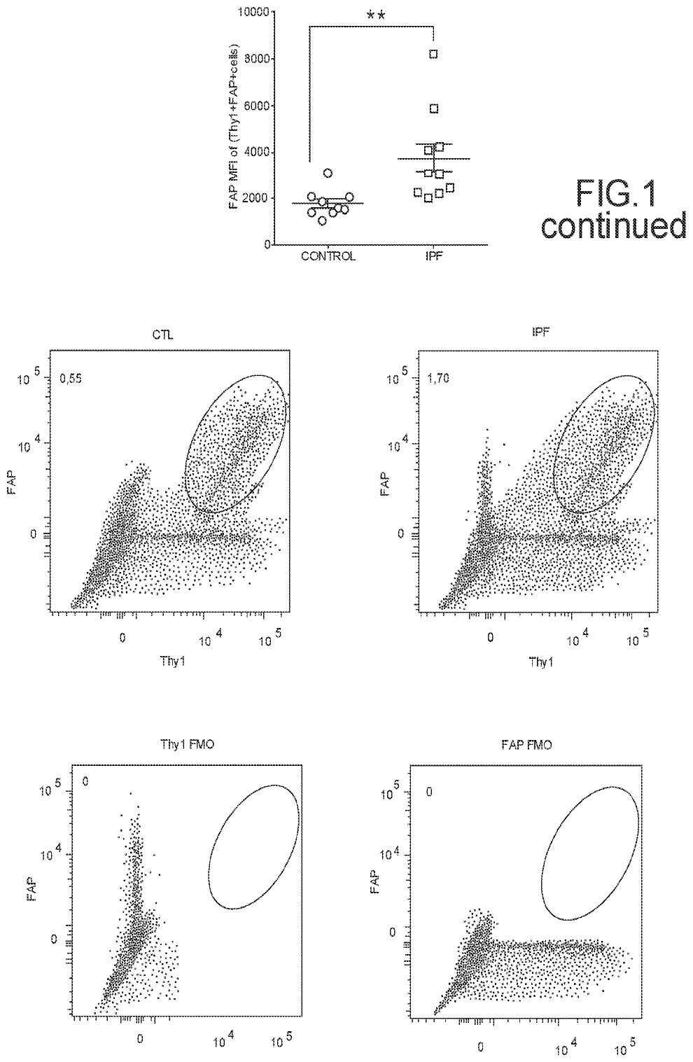

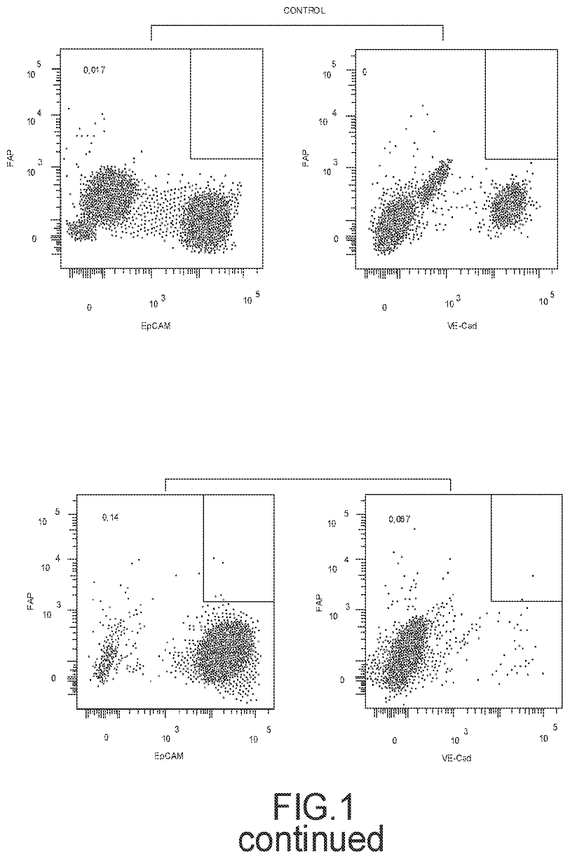

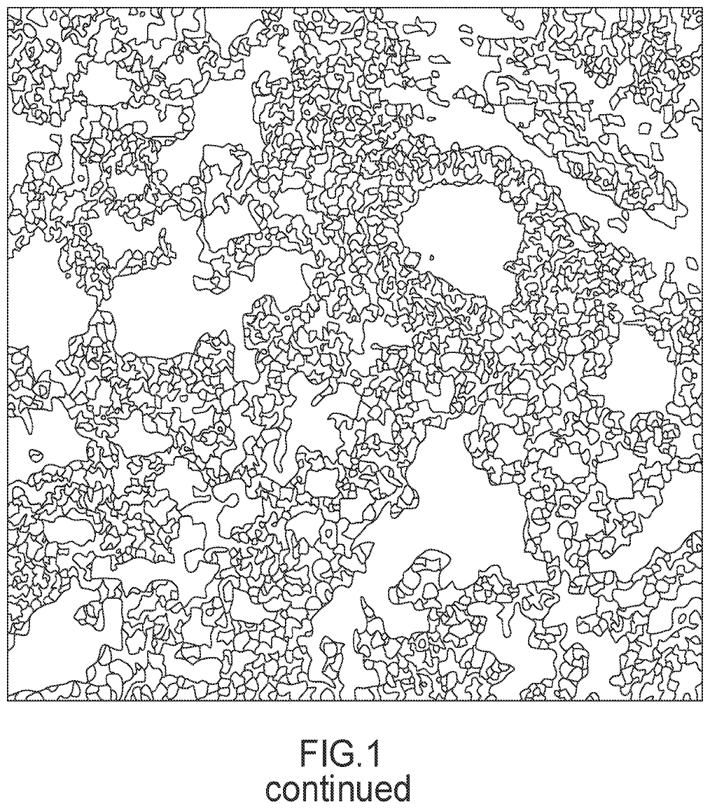

Based on reports in the literature that fibroblast activation protein (FAP) is upregulated in human IPF lung fibroblasts but largely absent from all other cell types, except cancer-associated fibroblasts and fibroblasts in tissues undergoing repair or remodeling, healthy and IPF human lung tissue were digested and the resulting cell suspensions were examined for expression of FAP. As shown in FIGS. 1A-1E, FAP is only expressed on lung fibroblasts in a manner that is strongly upregulated in fibrotic tissue. Thus, FAP was targeted for selective delivery of therapeutics to the activated subset of fibroblasts in IPF lungs.

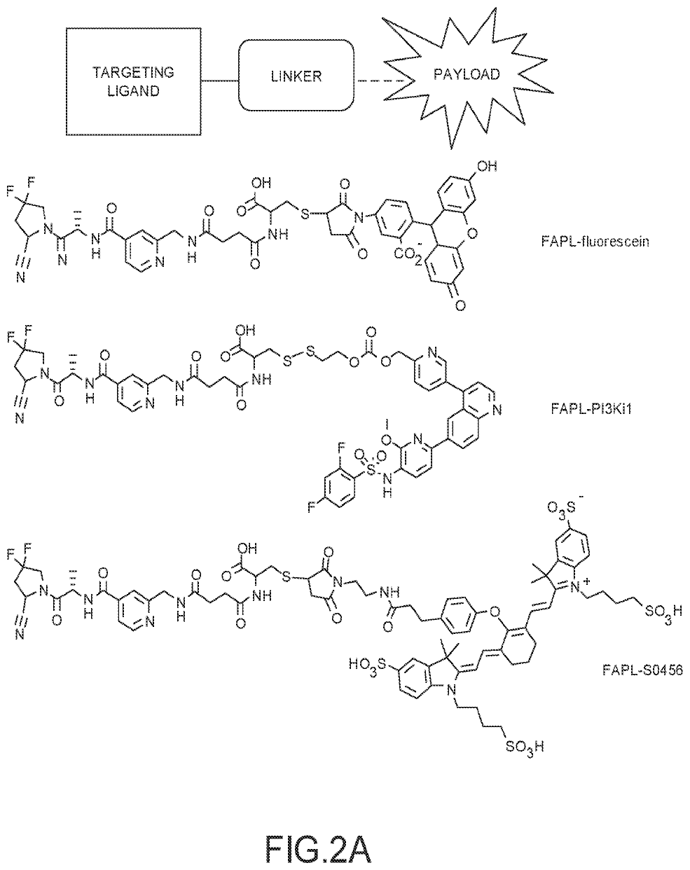

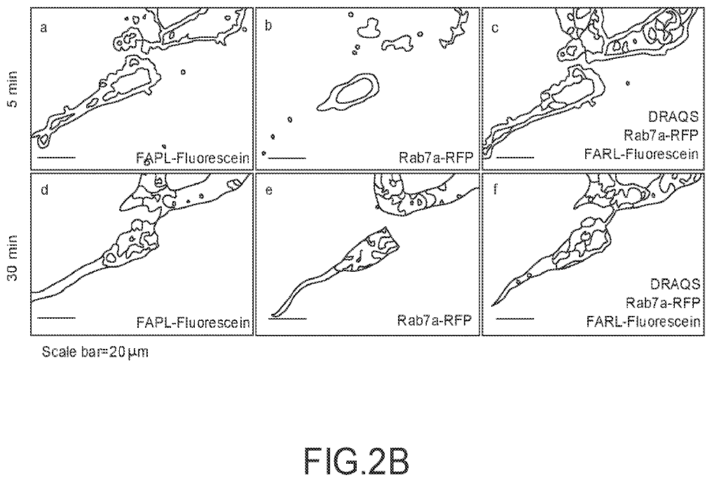

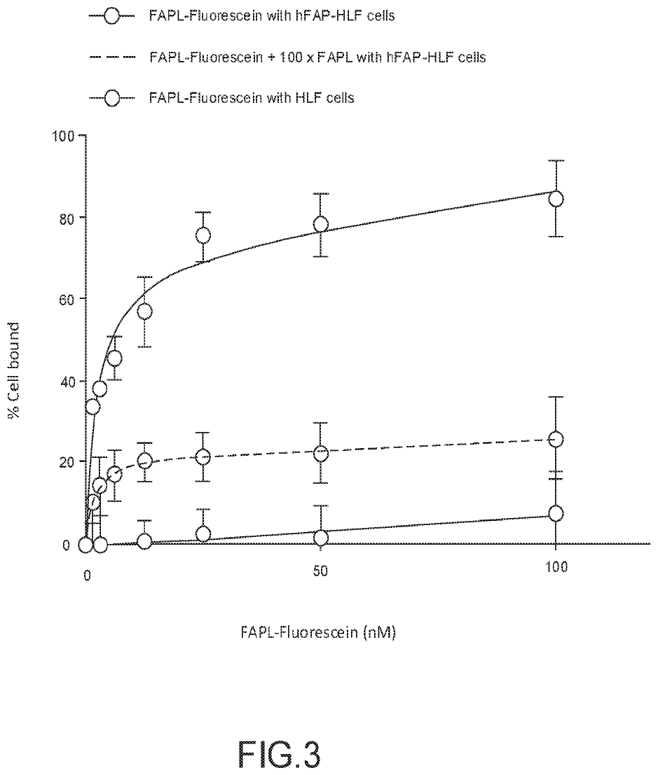

To test the ability of this ligand to target drugs to myofibroblasts in fibrotic tissues, a FAP-targeting ligand (FAPL) was linked to fluorescein (FIG. 2A, upper structure) and its interaction with a stable human lung fibroblast cell line with FAP-expression (HLF-FAP) was examined. As shown in the confocal micrographs of FIG. 2B, the fluorescent conjugate (FAPL-fluorescein) was found to bind HLF-FAP cells and rapidly internalize into Rab7a-expressing endosomes (compare 5 min vs. 30 mm time points). As further revealed in FIG. 3, the same fluorescein conjugate was demonstrated to bind FAP with high affinity (K_{d}˜10 nM) in a manner that could be largely prevented by co-administration of excess FAPL; confirming that binding was FAP-specific. Finally, as also revealed in FIG. 3, binding of FAPL-fluorescein to HLF cells not transfected with FAP was minimal, suggesting that induction of FAP expression was required for FAPL-fluorescein binding. Taken together, these data demonstrate that FAPL constitutes an attractive candidate for specific targeting of drugs to myofibroblasts in fibrotic tissues.

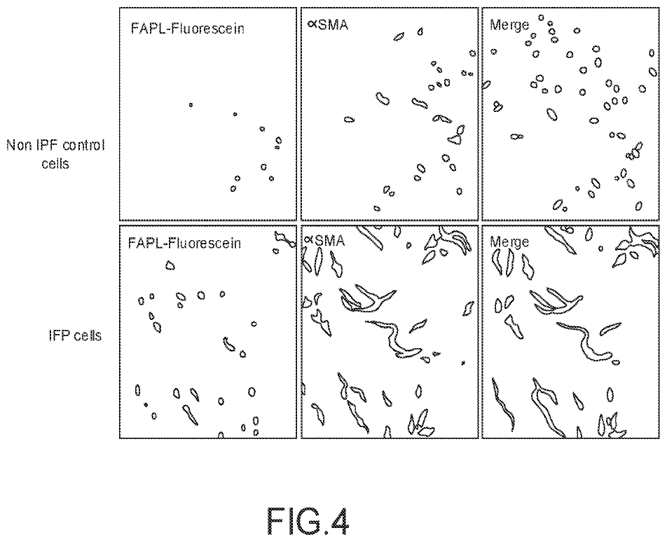

To determine whether FAPL-mediated drug delivery can occur in a more IPF-relevant cell type, FAPL-fluorescein uptake by primary human lung fibroblasts obtained from IPF patients was examined next. As shown in FIG. 4, FAPL-fluorescein binds to IPF fibroblasts (as confirmed by its colocalization with alpha smooth muscle actin (αSMA)), whereas little uptake is seen by control fibroblasts obtained from human lung explants. These data demonstrate that the FAPL can also deliver attached drugs to human IPF myofibroblasts.

Design and Synthesis of a phosphatidylinositol-3-kinase inhibitor for Inhibition of Collagen Synthesis

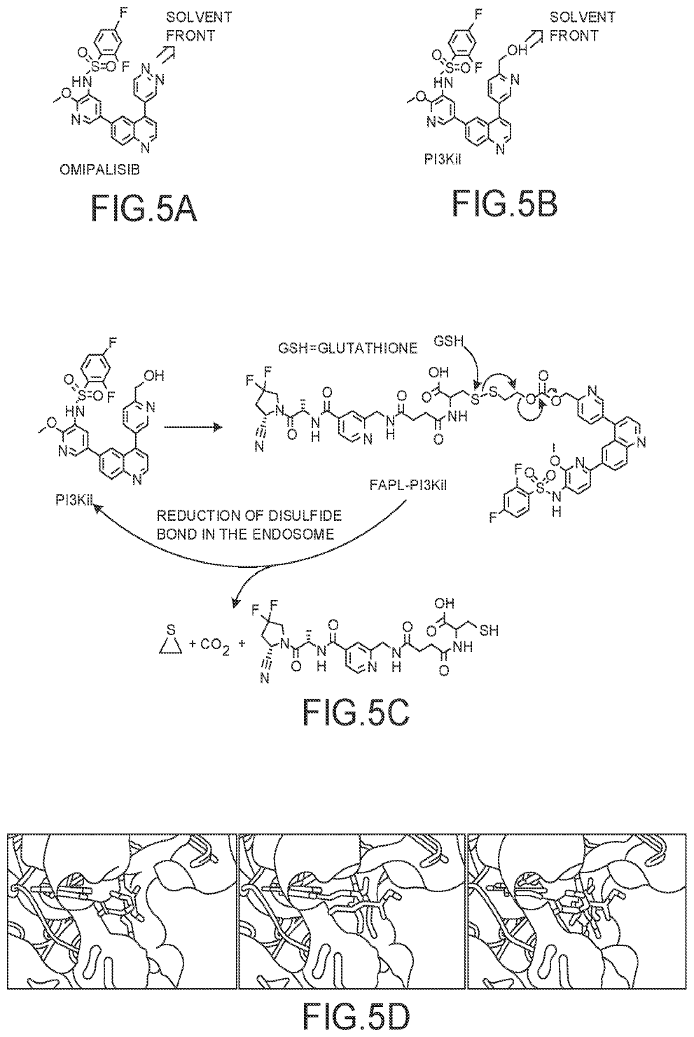

A PI3K inhibitor (PI3Ki1) that could be readily delivered into myofibroblasts with FAPL was designed. Although omipalisib, a PI3Ki recently introduced into IPF clinical trials lacked a functional group for conjugation to FAPL (FIG. 5A), a similar molecule to omipalisib was pursued that would retain its inhibitory potency but contain a functional group for facile conjugation to FAPL via a cleavable linker. The PI3Ki shown in FIG. 5B contains the modified omipalisib and the structure of its conjugate to FAPE is presented in FIG. 2A (middle structure). FIG. 5C then shows how reduction of the disulfide bond connecting FAPL to PI3Ki1 within an intracellular reducing environment can trigger self-immolative release of the unmodified PI3Ki1for inhibition of collagen synthesis. As shown in FIG. 5D (left panel), the difluorosulfonamide end of omipalisib is seen to fit well into the bottom of the catalytic site of PI3Kγ, allowing the quinoline end of the inhibitor to protrude into the aqueous space. However, as noted above, because the aqueous-exposed pyridazine cannot be derivatized with FAPL, it was converted into a pyridine-hydroxymethyl substituent, which was readily conjugated to FAPL. Surprisingly, this pyridine-hydroxymethyl modification not only did not obstruct binding of the inhibitor to PI3Ki, but actually enhanced the affinity of the modified inhibitor (PI3Ki1) for PI3Kγ.

### Evaluation of Myofibroblast Inactivation Using Targeted and Nontargeted PI3Ki1 In Vitro

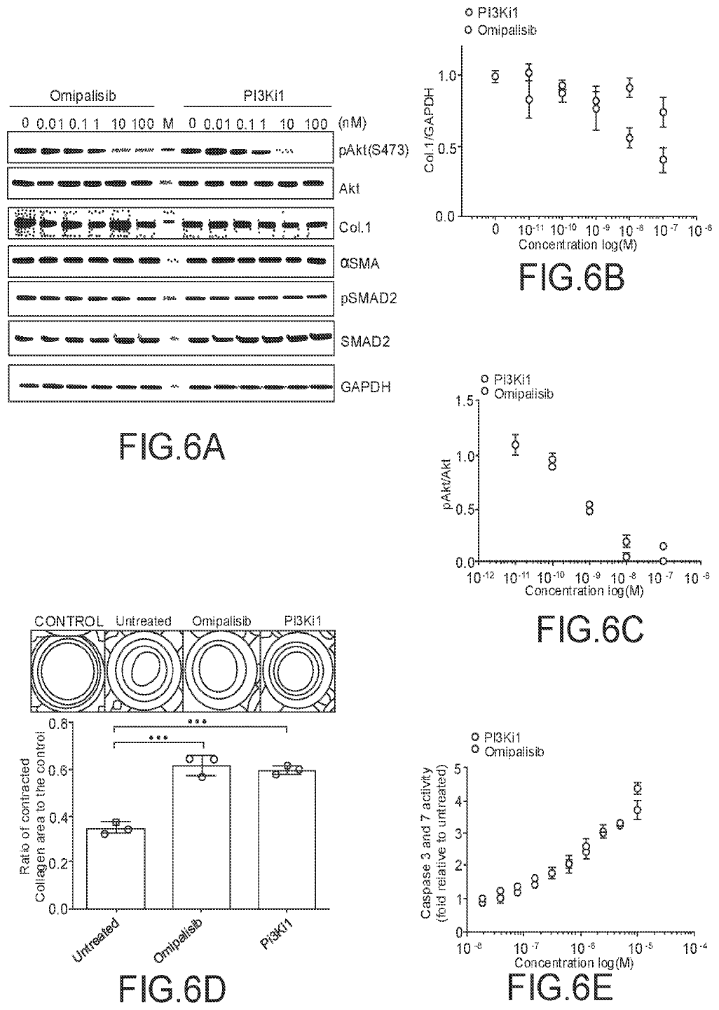

To determine whether the nontargeted version of PI3Ki1 might enter human lung fibroblasts and inhibit PI3K activity, HLF-FAP cells were incubated for 24 h with either omipalisib or PI3Ki1 and then the impact on TGFβ₁ stimulation, including phosphorylation of Akt, collagen synthesis, contraction of a collagen gel, and apoptosis of myofibroblasts were examined. As shown in the anti-phospho-Akt blots of FIG. 6A, nontargeted PI3Ki1 inhibited phosphorylation of Akt at least as well or better than omipalisib, displaying an IC₅₀˜1 nM and achieving nearly complete inhibition of Akt phosphorylation on serine 473 (pAkt^{S413}) by 10 mM concentration (FIG. 6B). Moreover, nontargeted PI3Ki1suppressed collagen synthesis with better potency than omipalisib, displaying an IC₅₀˜10 nM (FIG. 4C). Quantitation of the ability of PI3Ki1 to inhibit TGFβ₁ stimulated fibroblast contraction of a collagen gel further confirmed the ability of PI3Ki1 to reduce TGFβ₁ induced collagen remodeling (FIG. 6D). Finally, analysis of the impact of nontargeted PI3Ki1 on fibroblast apoptosis (e.g., caspase 3 and 7 activation) demonstrated that PI3Ki1 only promoted fibroblast cell death at concentrations much higher than those required to prevent collagen synthesis (FIG. 6E). This weak induction of caspase activity at PI3Ki1 concentrations below 100 nM suggests that a large therapeutic window exists between PI3Ki1 concentrations required to suppress fibrotic activity and those that cause cell death.

Because many PI-3 kinase inhibitors (including omipalisib) exhibit dose-limiting systemic toxicities in humans, it became important to determine whether inhibition of PI3K by the FAP-targeted PI3Ki1 conjugate (FAPL-PI3Ki1) might be restricted to FAPL-expressing cells, thereby limiting its toxicity to FAP-expressing cells. To examine this issue, three independent experiments were performed.

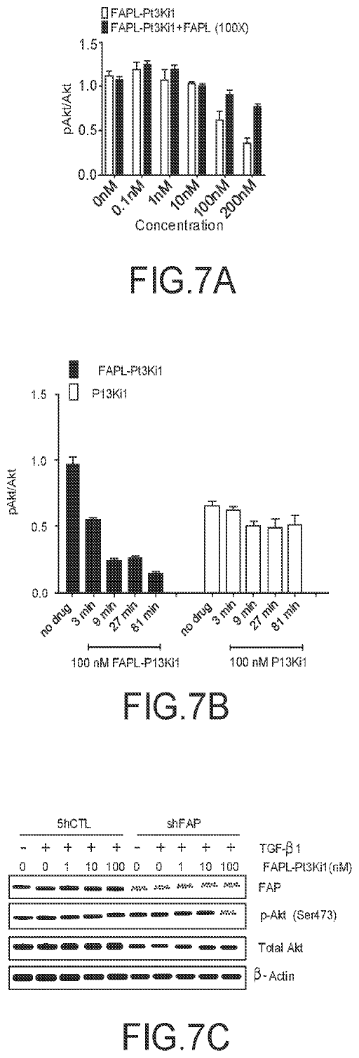

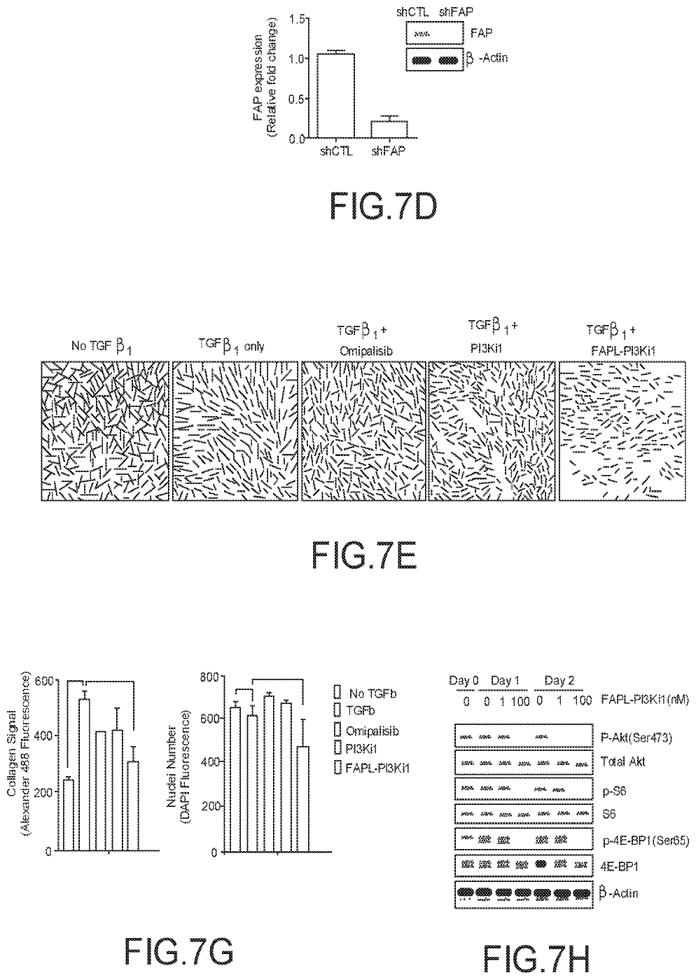

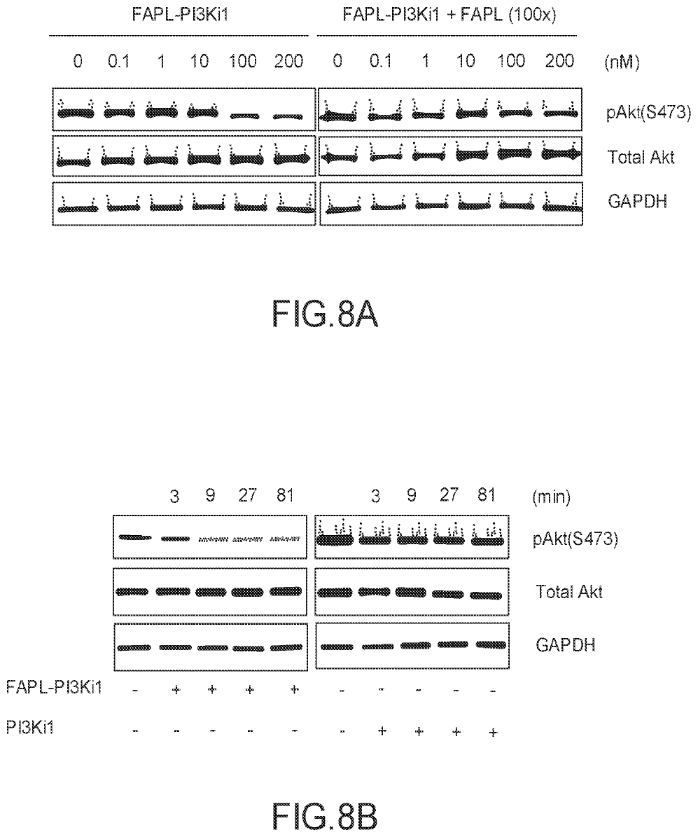

Firstly, primary lung fibroblasts from an IPF patient were stimulated for 24 h with TGFβ₁ (to activate them to a FAP-expressing state) and then incubated for 2 h with increasing concentrations of FAPL-PI3Ki1, in the presence or absence of 100-fold excess FAPL to block unoccupied FAP sites. As shown in FIG. 7A and FIGS. 8A-8B, phosphorylation of Akt was significantly inhibited by FAPL-PI3Ki1. This inhibition was reversed upon co-incubation with excess FAPL, demonstrating that FAPL-PI3Ki1 entry into IPF fibroblasts requires an unoccupied FAP on the fibroblast cell surface.

Secondly, IPF lung fibroblasts were stimulated with TGFβ₁ and then incubated for different durations with either FAPL-targeted or nontargeted PI3Ki1, followed by replacement of the culture media with inhibitor-free media (FIG. 7B and FIGS. 8A-8B). The anticipation was that FAP-targeted PI3Ki1 would be retained by FAP on FAP-expressing cells during short incubation times, whereas nontargeted PI3Ki1would not be captured by FAP and would subsequently be washed away when the media was changed. As shown in FIG. 7B, FAPL-PI3Ki1 showed a time-dependent reduction in phosphorylated Akt (pAkt), while nontargeted PI3Ki1 showed no diminution in pAkt expression up to the longest (81 min) incubation period.

Thirdly, FAP involvement in binding and internalization of PI3Ki1was documented by knocking down FAP in IPF lung fibroblasts using short hairpin RNA (shFAP) and examining the subsequent inhibition of Akt phosphorylation by FAR-PI3Ki1. As seen in FIGS. 7C and D. suppression of Akt phosphorylation is less in shFAP-treated than shRNA control-treated IPF lung fibroblasts (shCTL), especially at 1 and 10 nM. At 100 nM shRNA, nonspecific PI3Ki1 uptake seems to predominate in all samples. Taken together, these results demonstrate that FAP expression is required for uptake and FAPL-PI3Ki1mediated suppression of TGFβ₁-induced Akt activation in IPF fibroblasts.

Next, the FAP-targeted PI-3 kinase inhibitors were tested to determine if they might suppress collagen formation by human IPF fibroblasts. For this purpose, TGFβ₁-stimulated IPF lung fibroblasts were stimulated for 2 h with omipalisib, PI3Ki1, or FAPL-PI3Ki1, followed by replacement of the inhibitor-containing media with inhibitor-free growth media and continued incubation for 46 h. As shown in the collagen-stained micrographs of FIG. 7E and their quantitation in FIG. 7F, incubation with TGFβ₁ was required for stimulation of the biosynthesis of collagen, and this biosynthesis was only moderately inhibited by nontargeted PI3K inhibitors, but strongly inhibited by FAP-targeted PI3Ki1. Finally, because many PI3K inhibitors exhibit cross-inhibitory activity towards mTOR, it was determined whether FAPL-PI3Ki1 might also suppress phosphorylation of an established substrate of mTOR, namely 4E-BP1. As shown in panel G, phosphorylation of 4E-BP1 is indeed inhibited by FAPL-PI3Ki1, demonstrating that FAPL-PI3Ki1inhibits mTOR as well as PI3K. Importantly, this concurrent suppression of both mTOR and PI3K activity should be very beneficial to the desired therapy, since collagen synthesis associated with pathogenic fibrosis can be induced by both pathways.

### Evaluation of FAPL Targeting of a Fluorescent Dye to Fibrotic Lung Tissue in a Mouse Model of Pulmonary Fibrosis

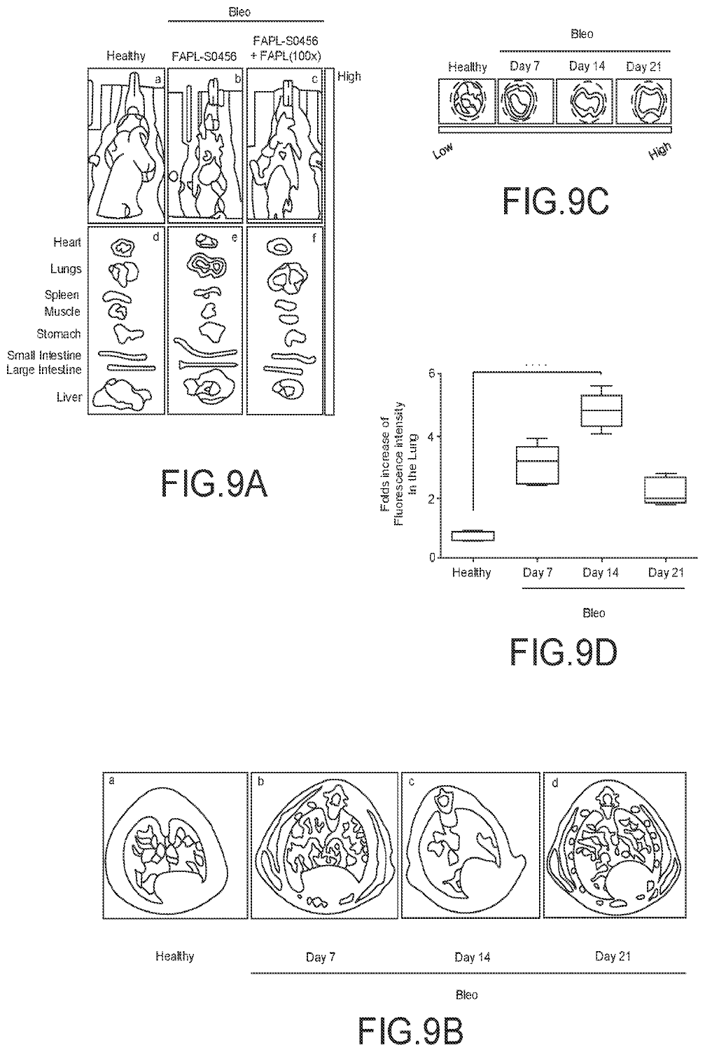

With the promising in vitro results obtained using both a human lung fibroblast cell line and primary human lung fibroblasts from an IPF patient, the possibility of using FAP to deliver a therapeutic drug to lung myofibroblasts in vivo was investigated. For this purpose, the bleomycin-induced lung fibrosis model in the mouse was exploited, in which a single intratracheal instillation of bleomycin (Bleo, 0.75 u/Kg) induces pulmonary fibrosis, including excessive interstitial deposition of collagen, proliferation of several lung cell types, infiltration of immune cells and contraction of alveolar spaces. To establish that this model also results in accumulation of FAP-expressing lung myofibroblasts, Bleo-instilled mice were injected via tail vein with 5 mmol of a FAPL-targeted near-infra red (NIR) dye (FIG. 2A, bottom structure, FAPL-S0456) and its uptake into affected lungs in the presence and absence of excess FAPL was compared. As shown in FIG. 9A, FAPL-S0456 accumulates specifically in the lungs of Bleo-treated mice, but not in the lungs of healthy mice. Moreover, uptake of FAPL-S0456 in the lungs of Bleo-treated mice can be blocked upon co-administration of excess FAPL—demonstrating that FAPL-S0456 uptake is dependent on both induction of fibrosis and the availability of unoccupied FAP receptors. Evidence that the severity of fibrosis was similar between Bleo-treated control and Bleo-treated competition groups was readily gleaned from data showing a similar amount of hydroxyproline accumulation in both treatment groups. Moreover, in agreement with the known spontaneous resolution of the pathology in this model after day 21 and congruent with the micro-CT data of FIG. 9B, uptake of FAPL-S0456 was absent in the lungs of healthy mice, moderate in the lungs of Bleo-treated mice at day 7 post-infusion, prominent in the same mice at day 14 post-infusion, and then moderate again in the mice at 21 days post-infusion (FIGS. 9C and D). Based on these data, it was concluded that this Bleo-induced fibrosis model in the mouse constitutes a valid system for testing the ability of a FAPL-targeted drug to treat a fibrotic lung disease in vivo.

### Evaluation of Myofibroblast Inactivation Following Administration of FAP-Targeted PI-3 Kinase Inhibitor In Vivo

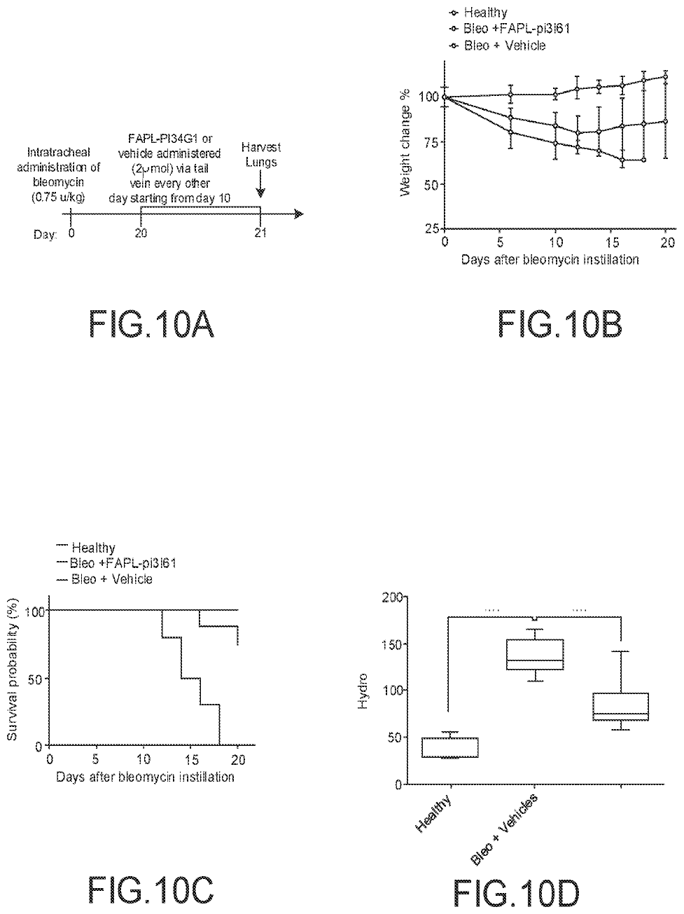

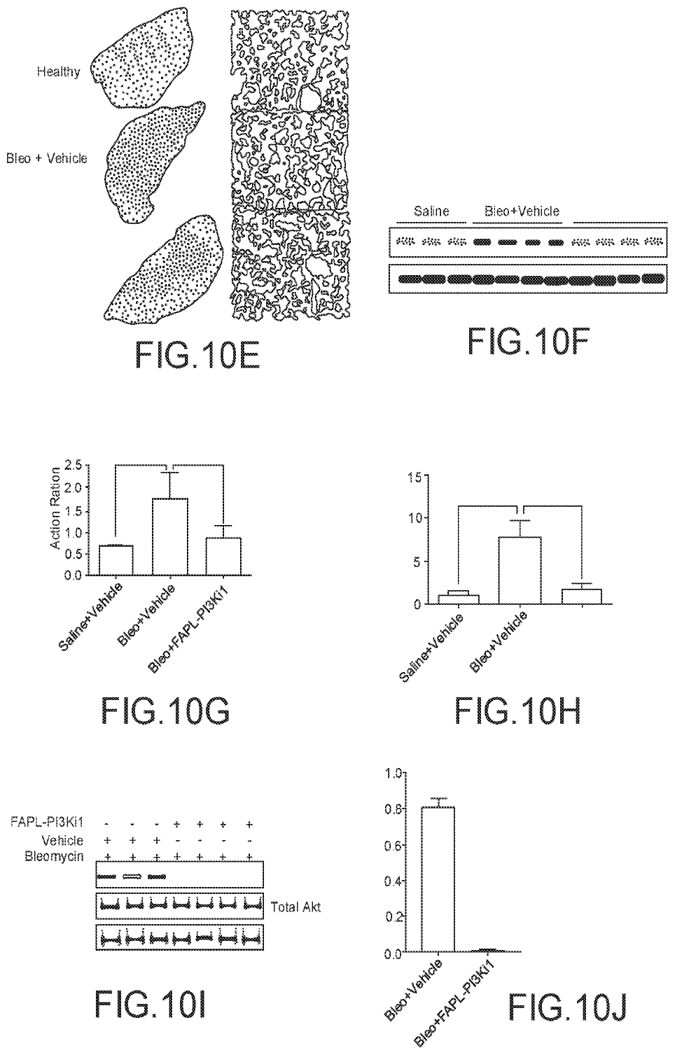

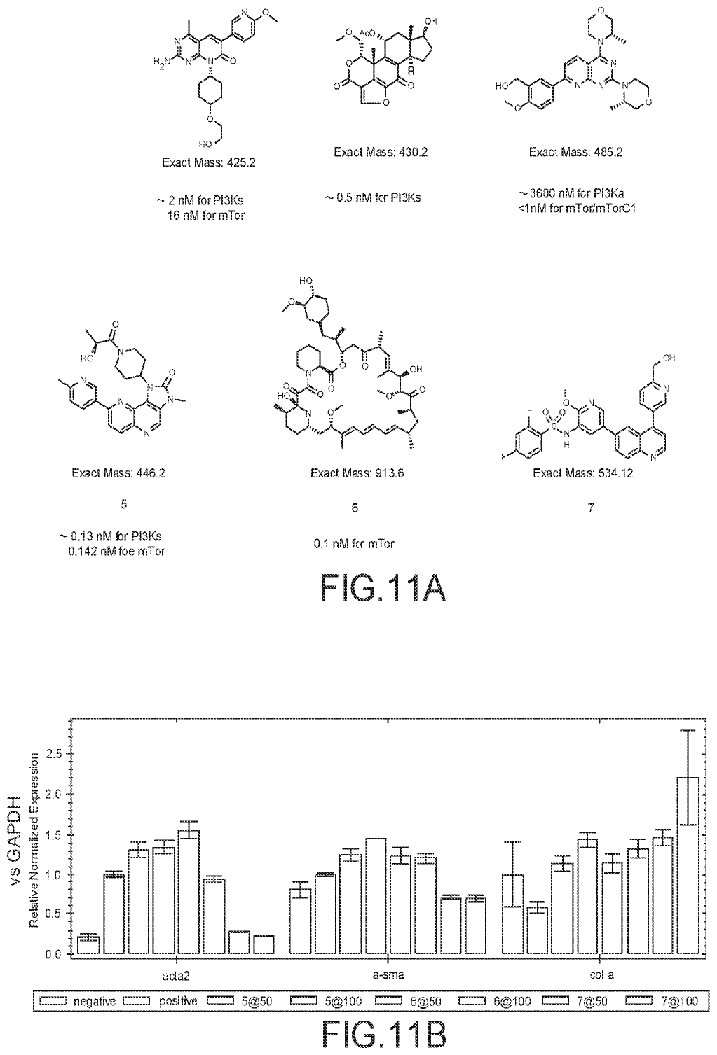

To investigate the therapeutic potential of fibrosis-targeted PI3Ki1 in vivo, mice were treated with bleomycin as described above and allowed to develop fibrosis prior to initiation of therapy on day 10 (FIG. 10A). Mice were then injected intravenously (tail vein) every other day with either saline or 2 μmol/kg FAP-PI3Ki1 and then sacrificed on day 21 for fibrosis analysis. As shown in FIG. 10B, Bleo-treated mice lost weight continuously from the moment of bleomycin instillation, presumably as a consequence of both bleomycin toxicity and progressive fibrosis. In contrast, FAPL-PI3Ki1treated mice lost weight only until day 12 (i.e., until 2 days after initiation of therapy), after which they gained weight continuously. Moreover, all 10 mice that did not receive FAPL-PI3Ki1 died prior to euthanasia on day 21, whereas only two of ten mice treated with FAPL-PI3Ki1 died before CO₂ euthanasia on day 21 (FIG. 10C). These data suggest that FAPL-PI3Ki1 therapy can mitigate the damage caused by instillation of bleomycin.

To obtain more mechanistic information on the molecular basis of the improved survival of the FAPL-PI3Ki1 treated mice, lungs from both saline and FAPL-PI3Ki1 treated mice were removed and analyzed for hallmarks of lung fibrosis. As shown in FIG. 10D, quantitation of hydroxyproline, a major component of collagen (i.e., the dominant biopolymer in fibrosis), was significantly elevated in mice treated with saline, but only marginally increased in mice treated with FAPL-PI3Ki1. This difference in collagen accumulation was confirmed by subjecting thin sections of the lungs to trichrome staining (a stain for collagen), which demonstrated significantly increased collagen deposition in the saline-treated group compared to the FAPL-PI3Ki1-treated groups (FIG. 10E). More detailed scrutiny of these same thin sections further revealed that the sizes and abundances of air sacs are markedly decreased in saline-treated mice compared to FAPL-PI3Ki1-exposed cohorts. As shown in FIG. 10F-H, evaluation of the lung homogenates from the different groups of mice showed significantly reduced α-SMA (panels F and G) and collagen 1A1 (panel H) in the FAPL-PI3Ki treated group. Taken together, these data demonstrate that administration of a FAP-targeted PI3K inhibitor suppresses the major markers of fibrosis in Bleo-treated mice.

Next, to confirm that the mechanism of FAPL-PI3Ki1 action involves inhibition of PI3K, a major signaling intermediate in the pathway for induction of collagen synthesis, lungs from Bleo-treated mice 2 h after intravenous injection of either saline or FAPL-PI3Ki1 were removed and their homogenates were immunoblotted with antibodies to Akt and phospho-Akt. As shown in FIG. 10I., treatment with FAPL-PI3Ki1 had no effect on the total amount of Akt (i.e., the immediate downstream substrate of PI3K) in the lung homogenates, confirming that the targeted drug is neither eliminating the fibroblasts nor promoting turnover of Akt. In contrast, treatment with FAPL-PI3Ki1 strongly reduced phosphorylation of Akt to its activated state (>95% reduction; FIG. 10J), confirming that the targeted therapy indeed engages its intended target and thereby blocks the primary signaling pathway for activation of collagen synthesis.

When considered together, the data presented above demonstrate that FAPL-PI3Ki1 suppresses fibrosis in Bleo-treated mice by inhibiting induction of collagen synthesis via the targeted blockade of PI3K, specifically in the fibrotic lungs of affected mice.

### FAPL-PI3Ki1Mitigates the TGFβ₁-Induced Pro-Fibrotic Phenotype and Collagen Deposition in Precision Cut Lung Slices (PCLS) From IPF Patients

Finally, to obtain an initial indication of the possible therapeutic benefit that might derive from treatment of human IPF patients with FAPL-PI3Ki1, PCLS from resected lungs of IPF patients were prepared, and the effect of incubation for 72 hours in media containing or lacking 100 nM FAPL-PI3Ki1 was examined. The FAP-targeted inhibitor strongly suppressed production of collagen. Moreover, when expression of collagen 1A1 and other markers of fibrosis (fibronectin and alpha-smooth muscle actin) were quantified by qPCR, FAPL-PI3Ki1treatment was confirmed to inhibit transcription of these other hallmarks of fibrosis. Collectively, these data argue that FAPL-PI3Ki1 displays significant potential for also mitigating the symptoms of IPF in humans.

### Discussion

Although the causes of fibrosis can be many, virtually all fibrotic processes seem to involve activation of fibroblasts to myofibroblasts and their subsequent over-production of collagen. Based on this commonality and the fact that myofibroblasts are only found in healing wounds, solid tumors, and fibrotic tissues. it seemed prudent to i) design a method that would target drugs specifically to myofibroblasts in vivo, and then ii) use the method to deliver collagen synthesis inhibitors selectively to the collagen-synthesizing myofibroblasts. Except in fibrosis patients suffering simultaneously from cancer or tissue trauma, such a targeted approach should be specific for fibrotic tissue, thereby avoiding any collateral toxicity that might arise when effective drugs are taken up by healthy tissues.

The compounds described herein are targeted to FAP because FAP is upregulated whenever a fibroblast is activated to become collagen-producing and in some epithelial cells undergoing an epithelial to mesenchymal transition. PI3K inhibitors were chosen because PI3K is central to most pathways involved in induction of collagen synthesis and since a nontargeted PI3K inhibitor is currently undergoing human clinical trials for treatment of IPF. The fact that i) FAP-expressing myofibroblasts are critical to development of IPF, ii) our FAP targeting ligand binds human FAP with high specificity and affinity, and iii) collagen production in primary human IPF lung fibroblasts is potently inhibited by FAPL-PI3Ki1 argues strongly that production of collagen by human myofibroblasts in IPF patients can also be suppressed by FAPL-PI3Ki1. And there does not appear to be any prior report of any myofibroblast-targeted therapy capable of delivering an anti-fibrotic drug selectively to the cells responsible for fibrosis.

While a number of therapeutic “warheads” could have been selected for delivery with FAPL, the question naturally arises why a pan PI-3 kinase inhibitor was chosen in view of the prior toxicities associated with systemic administration of more isozyme-specific PI3K inhibitors. Thus, the PI3K/Akt/mTOR signaling pathway mediates a variety of critical cellular processes, including cell cycle progression, growth and proliferation, metabolic and synthetic pathways, and a number of inflammatory responses. Although systemic suppression of these pathways would logically be expected to cause systemic toxicity, when a drug can be targeted to the pathological cell, concern over systemic toxicities declines, because the drug is concentrated in the diseased cells and excluded by the healthy cells. With this capability, use of a pan PI3K inhibitor becomes an advantage, since it should avoid problems deriving from leak-through collagen synthesis that arises when minor forms of PI3K become activated.

### Materials and Methods

### Study Design

Although PI-3 kinase/mTOR inhibitors have been successfully employed to inhibit fibrosis in preclinical animal models, no PI-3 kinase/mTOR inhibitor has yet been approved for fibrotic applications in humans due to unacceptable off-target toxicities. To determine whether such toxicities could be mitigated by specific targeting of a PI-3 kinase/mTOR inhibitor to myofibroblasts (i.e., the cells that cause fibrosis), a myofibroblast-targeting ligand was designed and then its ability to deliver attached drugs selectively to fibrotic lung myofibroblasts in a bleomycin-induced murine pulmonary fibrosis model was tested. To validate the ability of this novel targeting ligand to concentrate attached drugs specifically in fibrotic tissue, its ability to localize a fluorescent dye in the lungs of mice with bleomycin-induced pulmonary fibrosis was examined first. The ability of the same targeting ligand to deliver an attached PI-3 kinase/mTOR inhibitor to the myofibroblasts of these fibrotic lungs was then evaluated by quantitating the suppression of multiple fibrotic markers. Included among these markers were alpha smooth muscle actin (a myofibroblasts-specific marker), collagen 1A1, hydroxyproline, fibronectin, the mRNA for alpha smooth muscle actin and the mRNA for collagen 1A1. In all cases, the changes in these markers were quantitated in both treated and untreated lungs of bleomycin-induced mice as well as in lungs from healthy mice.

To ensure statistical significance in all these studies, preliminary experiments were performed to determine the number of mice per treatment group that would be required to achieve a P<0.05 in one way ANOVA tests. These initial studies demonstrated that at least 10 mice/group were needed to achieve statistical significance. Moreover, to ensure that the myofibroblasts-targeted therapy would indeed address all major symptoms of pulmonary fibrosis, multiple disease-related signal transduction intermediates were monitored to ensure that each major fibrosis pathway would be inhibited by FAP-PI3Ki1. These other fibrosis-related signaling intermediates included phospho-Akt, ribosomal protein S6, the transcription factor 4E-BP1 and SMAD2.

All in vitro experiments were performed in triplicate on separate days to ensure reproducibility. In the case of animal studies, mice were random/zed according to their body weights before the start of treatments to eliminate any weight-related bias. No samples or animals were ever excluded from data analysis for any reason. In vivo experiments were terminated 21 days after instillation of bleomycin, because bleomycin-induced fibrosis is known to begin to resolve spontaneously after that time point. All statistical methods are described in the “Statistical analysis” section.

### Cell Culture and Animal Husbandry

IPF patient cell lines were obtained from subjects who provided informed consent and underwent lung transplantation, control fibroblasts were obtained from donor organs. C57BL6/6-NCrl (Strain code: 027) mice were purchased from Charles River and maintained on normal rodent chow. Mice were housed in a sterile environment on a standard 12 h light-and-dark cycle for the duration of the study. All animal procedures were approved by the Purdue Animal Care and Use Committee (PACUC) in accordance with NIH guidelines.

### Flow Cytometry Analysis and Stain in of Human Lung Tissue Samples

Human lung tissue samples were obtained from Brigham and Women's Hospital from patients diagnosed with terminal fibrotic lung disease/IPF, i.e., those who underwent lung transplants. Control lungs had no evidence of chronic lung disease and/or histological evidence of fibrosis. Tissue digests for flow cytometry were carefully selected by pulmonologists based on biopsy report and CT scans and demonstrated a clear manifestation of the disease of interest. Tissues were initially digested into single cell suspensions and bio-banked in the biorepository. At the time of flow cytometry, single cell lung digests were thawed in media and placed in PBS containing 0.1 mg/ml DNAse I solution (Stem Cell technologies, Cat #07900) to digest DNA from dead cells and prevent cell clumping. Cells were then filtered to remove clumps/debris, counted, and 1-2 million cells were prepared for flow cytometry staining and analysis. Cells were initially stained with a Zombie live/dead viability dye (BioLegend, Cat #423101) in PBS for 30 minutes at room temperature. Samples were then washed with FACS buffer (0.3% BSA in PBS) and stained with Human TruStain FcX (BioLegend, Cat #422301) for 15 minutes to prevent unwanted staining of Fc receptors. Samples were subsequently stained with an antibody cocktail mix in FACS buffer containing, anti-human CD45 (APC Fire 750, BioLegend, Cat #368518), anti-human CD90/Thy1 (APC, BioLegend, Cat #328114), anti-human FAP (PE, R&D Systems, Cat #FAB3715P), anti-human CD326/EpCAM (PE Cy7, eBioscience, Cat #25-9326-42) and anti-human CD144/VE-Cad (BV421, BD Horizon, Cat #565671) for 30 minutes at 4° C. Finally, samples were washed twice, resuspended in FACS buffer and examined using a BD LSRFORTESSA cell analyzer. Data were analyzed using FLOWJO version 10.2.

### Live Cell Imaging of FAPL-Fluorescein Internalization

HLF-hFAP cells were seeded in a glass-bottom dish and incubated overnight with endosome tracker (Rab7a-RFP, ThermoFisher). Cells were then incubated with FAPL-Fluorescein (10 nM) for 1 hour at 4° C., followed by staining with 5 nM DRAQ5 nuclear dye (ThermoFisher). After washing 3 times in PBS washes, spatial localization of FAPL-Fluorescein was monitored at any given time under ambient temperature by confocal microscopy (FV 1000, Olympus). Confocal images were further processed using FV10-ASW Olympus software.

### Immunofluorescence of FAP and αSMA Expression in Fibroblasts

HLF cells, primary human IPF fibroblasts and non-IPF fibroblasts were cultured, fixed, and permeabilized on glass-bottom dishes for immunofluorescent staining. Primary antibodies against hFAP (1:200, FAB3715R, R&D Systems) or αSMA (1:1000, ab21027, Abcam) were incubated overnight at 4° C. After PBS washes, samples were incubated with Alexa Fluor® 488-labeled secondary anti-goat antibodies (Abcam, 1:400). Images were captured and analyzed by confocal microscopy.

### Western Blot Analysis of Cultured Fibroblasts

Serum starved confluent HLF cells were co-incubated in medium containing 10 ng/ml TGFβ₁ with or without the indicated concentrations of PI3K inhibitors for 24 hours. Cells were harvested and lysed for Western blot analysis. Following sodium dodecyl sulphate polyacrylamide gel electrophoresis and blocking, membranes were incubated with antibodies to detect pSMAD2 Ser465/467 (#3101, Cell Signalling Technology), or pAkt Ser473 (#4060, Cell signalling Technology), and signals were visualized with ECL Western Blot Detection Reagents (GE Healthcare). Following stripping, membranes were blocked and re-probed with antibodies specific for total SMAD2 (#3103, Cell Signalling Technology) or total Akt (#4060Cell Signalling Technology).

### Molecular Crowding Assay for Collagen

Confluent IPF fibroblasts (4000 cells/well) were cultured in 96-well plates in DMEM containing 0.4% fetal calf serum, ascorbic acid (100 μM), and mixed Ficoll 70 and Ficoll 400 as molecular crowding agents. Fibroblasts were stimulated with TGFβ₁ (10 ng/ml) and incubated with either vehicle (0.1% DNISO) or 100 nM of omipalisib, FAPL-PI3Ki1, or PI3Ki1 for 2 hours, followed by removal of media. Cells were then stimulated with inhibitor-free media containing TGFβ₁ (10 ng/ml) for 48 hours. Cells were fixed and stained with antibody specific for human collagen 1 and counterstained with fluorescent secondary antibody (Alex Fluo 488) Nuclei were counterstained with DAPI for cell counting on a high content system (Opera Phenix High Content Screening System, PerkinElmer),

### Precision-Cut Lung Slides

All the procedures were performed under sterile conditions. Bronchoalveolar lavage was performed twice to get rid of any blood coagulation. Pre-warmed agarose (Sigma, A0701, St. Louis, Mo.) was injected to lung explants through trachea until fully inflated. The inflated lung explants were placed on ice for 30 minutes to solidify the agarose. Tissue cylinder was made using tissue punch biopsy needle of 10 min diameter. Lung slides (350 μM) were prepared with VF-300-0Z Vibratome (Precisionary instruments, Natick, Mass.). The slides were cryopreserved in DMEM with 10% FBS and 10% DMSO.

### Collagen Immunofluorescence Staining

Precision-cut lung slides were put into 24-well slides and incubated with MAXblock Blocking Medium (Active Motif, Carlsbad, Calif.) 1 hour at 37° C. Slides were washed with 1× MAXwash Washing Medium (Active Motif, Carlsbad, Calif.) 10 minutes on a rotating platform twice. Primary anti-collagen antibody (Sigma, SAB4200678, St. Louis, Mo.) was incubated with slides at 1:500 dilution for 1 hour at 37° C., Next, slides were washed with 1× MAXwash Washing Medium (Active Motif, Carlsbad, Calif.) 10 minutes on a rotating platform thrice. Second antibody (Sigma, A28180, St. Louis, Mo.) was diluted at 1:1000 and incubated with slides for 1 hour at 37° C. Slides were washed with 1× MAXwash Washing Medium 10 minutes on a rotating platform five times. Slides were transferred to glass slides and mounted with mounting medium (Sigma, P36934, St. Louis, Mo.). Images were taken using FLUOVIEW FV10i. (Olympus, Center Valley, Pa.).

### Quantitative PCR

RNA was extracted by using TRIzol based on manufacture's specification (Invitrogen, 15596026, Waltham, Mass.). Extracted RNA was incubated with DNase I (Invitrogen, 18068-015, Waltham, Mass.) for 15 minutes at room temperature followed by DNase I deactivation. cDNA was synthesized by using Superscript IV according to manufacturer's specification (Invitrogen, 18091050, Waltham, Mass.). Cyber green supermix was used to perform Quantitative PCR (Bio-rad, 1725121, Hercules, Calif.). The primers are listed in the table as below.

| | |
|---|---|
| 18S rRNA forward | GCTTAATTTGACTCAACACGGGA |
| | [SEQ ID NO: 1] |
| 18S rRNA reverse | AGCTATCAATCTGTCAATCCTGTC |
| | [SEQ ID NO: 2] |
| Human a-SMA | CAGGGCTGTTTTCCCATCCAT |
| forward | [SEQ ID NO: 3] |
| Human a-SMA | GCCATGTTCTATCGGGTACTTC |
| reverse | [SEQ ID NO: 4] |
| Human Collagen 1 | AGC CAGCAG ATC GAG AAC AT |
| forward | [SEQ IDN O: 5] |
| Human Collagen 1 | TCC TTG GGG TTC TTG CTG AT |
| reverse | [SEQ ID NO: 6] |
| Human Fibronectin | ACTGTACATGCTTCGGTCAG |
| forward | [SEQ ID NO: 7] |
| Human Fibronectin | AGTCTCTGAATCCTGGCATTG |
| reverse | [SEQ ID NO: 8] |
| Mouse Collagen 1 | GGTGAACGTGGTGCAGCT |
| forward | [SEQ ID NO: 9] |
| Mouse Collagen 1 | TCTTTACCAGGAGAACCATCAG |
| reverse | [SEQ ID NO: 10] |
| Mouse Fibronectin | CTTTGGCAGTGGTCATTTCAG |
| forward | [SEQ ID NO: 11] |
| Mouse Fibronectin | ATTCTCCCTTTCCATTCCCG |
| reverse | [SEQ ID NO: 12] |

### Bleomycin-Induced Lung Fibrosis Model

Eight to 10-week old C57BL/6-NCrl (Strain Code: 027) male mice (Charles River) were anesthetized (mixture of xylazine/ketamine) and then injected intratracheally with freshly prepared 0.75 μ/Kg of bleomycin sulfate (Cayman Chemicals, Cat N13877) in sterile phosphate-buffered saline (PBS; volume was varied between 88-108 mL depending on the body weight), Control mice were injected with 50 μL of sterile phosphate-buffered saline. Body weights were monitored throughout each study. To quantitate FAP expression and fibrosis during longitudinal studies, lungs were harvested at 7, 14- and 21-days post-bleomycin instillation and assayed as described below. For therapy studies, induction of IPF was initiated as described above and drug (2 μmol/kg) was intravenously injected every other day beginning on day 10. Lungs were harvested on day 21 and assayed as described below (Day 0 was taken as the day of bleomycin administration).

### Western Blot Analysis of Lung Tissue

Frozen lungs were lysed in 1 ml of lysis buffer containing a protease inhibitor cocktail using an Ultra-thurrax, Lysates were cleared by centrifugation before total protein determination using the BCA protein assay. SDS-PAGE and Western blotting were performed following standard procedures. Membranes were blocked and then probed with antibodies directed against GAPDH, phosphorylated Akt, and total Akt. The membranes were then washed in Tris-buffered saline/Tween 20 followed by incubation with horseradish peroxidase-conjugated secondary antibodies. Immunoreactive bands were detected by addition of an enhanced chemiluminescence substrate.

### Hydroxyproline Assay

Total lung collagen was determined by analysis of hydroxyproline as previously described. The right lung was consistently set aside for this assay. Briefly, harvested right lung was homogenized in PBS (pH 7.4), digested with 12 N HCl at 120° C. for 3 hr. Citrate/acetate buffer (pH 6.0) and chloramine-T solution were added at room temperature for 20 minutes and the samples were incubated with Ehrlich's solution for 15 min at 65° C. Samples were cooled to room temperature and read at 550 nm. Hydroxyproline standards (Sigma, MO) at concentrations between 0 and 400 μg/ml were used to construct a standard curve.

### Histopathological Evaluation at Pulmonary Fibrosis

The left lung was inflated and fixed with 10% formalin solution (neutral buffered). Lung tissues were embedded in paraffin, and 10-μm sections were prepared and stained using H&E and Masson Trichrome stain. The severity of bleomycin-induced fibrosis was determined by semiquantitative histopathological scoring at the indicated dates after bleomycin administration.

### In Vivo Fluorescence Imaging

Mice were treated via tail vein injection with 5 nmol of FAP targeted NIR dye conjugate (FAPL-S0456) and imaged 2 hr post-injection using a Spectral AMI optical imaging system. For competition experiments, a 100-fold excess of the FAP ligand was co-administered with FAPL-S0456. The settings were as follows: Object height, 1.5; excitation, 745 nm; emission, 790 nm; FOV, 25; binning, 2; f-stop, 2; acquisition time, 1 s. After whole-body imaging, animals were dissected, and selected organs were collected and imaged again for complete biodistribution analysis. The conditions remained the same as those used in the longitudinal imaging study, except the mice were imaged on day 7, day 14, and day 21 post-bleomycin administration.

### Micro-CT Imaging

Micro-CT analysis of whole excised lung was performed on day 7, day 14, and day 21 post-bleomycin administration. Briefly, animals were anesthetized with isoflurane and fixed in prone position. Micro-CT images were acquired on a Quantum FX micro-CT system (Perkin Elmer, Waltham, Mass.) with cardiac gating (without respiratory gating), using the following parameters: 90 kV; 160 μA; FOV, 60×60×60 mm; spatial resolution, 0.11 mm, resulting in a total acquisition time of 4-5 minutes.

### Pharmacokinetic Analysis

2 μmol/kg of FAPL-PI3Ki1 was intravenously injected into healthy C57BL/6-NCrl mice and blood was collected at 5, 10, 15, 20, 25, 30, 60, 120, 180, 240, and 300 nun post injection. Samples were centrifuged at 1,000 g for 10 min and plasma was collected and treated with acetonitrile (plasma/acetonitrile=1/3 (v/v). After vortexing and then centrifuging at 1,000 g for 5 min, the supernatant was collected and injected into an Agilent 6410 NanoLC QQQ liquid chromatography-mass spectrometry (LCMS) for quantitation of FAPL-PI3Ki1concentration. Column: Agilent Eclipse Plus C18, 2.1×50 mm, SN: B17477. Eluent: A: water +0.1% formic acid, B: acetonitrile +0.1% formic acid. The PK data are reported in FIG. S11A.

### Stability Analysis

10 μl of 5 mM FAPL-PI3Ki1 were added to 100 μl of plasma obtained from healthy C57BL/6-NCrl mice and incubated at 37° C. for 3, 5, 10, 15, 20, 30, 40, 50, 60, 90, or 120 min. Samples were then extracted with acetonitrile and analyzed as described above. The concentration of FAPL-PI3Ki1 and PI3Ki1 as a function of time is shown in FIG. S11B.

### Statistical Analysis

Statistical analyses were performed with GraphPad Prism 7 software. A one-way ANOVA followed by post-hoc Tukey test was used for analyzing differences between treatment groups. Error bars represent means±SD as denoted in the figure legends. Statistically significant P values are indicated in figures and/or legends. A P value of <0.05 was considered significant.

### Synthetic Methods

### Experimental Procedure for the Synthesis of FAIT, FAIT-PI3Ki1, FAPL-Fluorescein, and FAPL-S0456

H-Cys(Trt)-2-Cl-Trt resin and protected amino acids were purchased from Chem-Impex Intl. 2-(Hydroxymethyl)pyridine-5-boronic acid, pinacol ester was purchased from Combi-Blocks. 6-bromo-4-iodoquinoline, 2-4-Difluorobenzene-1-sulfonyl-chloride and 5-bromo-2-methoxypyridine-3-amine were obtained from ArkPharm. All the other chemicals were purchased from SIGMA-Aldrich or Fisher Scientific. Thin layer chromatography (TLC) was carried out on Merck silica gel 60 F254 TLC plates. Silica gel column chromatography was performed using silica gel (60-120 μm particle size). Preparative reverse-phase high performance liquid chromatography (RP-HPLC) was performed on a Waters, XBridge™ Prep C18, 5 μm; 19×100 mm column, mobile phase A=20 mM ammonium acetate buffer, pH 5 or 7, B=acetonitrile, system with gradients in 30 min, 13 mL/min, λ=254/280 nm. LRMS-ESI (LCMS) was obtained using an Agilent LCMS 1220 system, with Waters, XBridge™ RP1.8, 3.5 μm; 3×50 mm column, mobile phase A=20 mM ammonium bicarbonate buffer, pH 5 or 7, B=acetonitrile, system with gradients in 12-15 min, 0.75 mL/min, λ=254/280 nm. The high-resolution mass measurements were recorded on a LTQ Orbitrap XL mass spectrometer utilizing electrospray ionization (ESI).

### Synthesis and Characterization

### Compound 3

2-4-Difluorobenzene-1-sulfonyl-chloride 2 (1 eq) was added slowly to a cooled solution of 5-bromo-2-methoxypyridine-3-amine 1 (1 eq) in pyridine. Reaction was stirred at ambient temperature for 16 h, at which time the reaction was diluted with water and solids were filtered off and washed with copious amounts of water. The precipitate was dried in high vacuum to give compound 3, which was used in the next step without further purification (30% yield). LRMS-LCMS (m/z): [M+H]⁺ calcd for C₁₂H₉BrF₂N₂O₃S, 377.9; found 378.9). ¹H NMR (500 MHz, Chloroform-d) δ 7.91-7.85 (m, 2H), 7.85-7.79 (d, J=1.9 Hz, 1H), 7.25-7.17 (s, 1H), 7.04-6.89 (m, 2H), 3.89 (s, 3H).

### Compound 5

A mixture of bis(pinacolato)diboron 4 (1 eq), compound 3 (1 eq), Pd(dppf)₂Cl₂ (0.1 eq), KOAc (3 eq) in anhydrous 1,4-dioxane was deoxygenated by bubbling with nitrogen for 10 min. The mixture was then heated at reflux for 3 h. After cooling to room temperature, the mixture was evaporated under reduced pressure and the residue was dissolved in EtOAc, washed with water twice and dried over magnesium sulfate. The crude product was purified by flash chromatography (Hex: EtOAc) to yield compound 5 (68% yield). LRMS-LCMS (m/z): [M+H]⁺ calcd for C₁₈H₂₁BF₂N₂O₅S, 426.1; found 427.1). ¹H NMR (500 MHz, Chloroform-d) δ 8.29-8.18 (d, J=1.6 Hz, 1H), 8.08-7.97 (d, J=1.7 Hz, H), 7.96-7.79 (m, 1H), 7.15-7.01 (s, 1H), 6.99-6.86 (m, 2H), 3.89 (s, 3H), 1.39 —1.33 (s, 12H).

### Compound 8

6-Bromo-4-iodoquinoline 6 (212.37, 0.636 mmol) and 2-(hydroxymethyl)pyridine-5-boronic acid 7 (150 mg, 0.636 mmol) were dissolved in anhydrous 1,4-dioxane (15 mL). To this was added Pd(dppf)₂Cl₂ (19.9 mg, 0.024 mmol) followed by 2M Na₂CO₃ (2.5 mL). The mixture was then heated at reflux for 6 hrs. After cooling to room temperature, the solids were filtered off and evaporated. The crude product was purified by flash chromatography (EtOAc:MeOH) to yield compound 8 (32% yield). LRMS-LCMS (m/z): [M+H]⁺ calcd for C₁₅H₁₁BrN₂O, 314; found 315). ¹H NMR (500 MHz, Methanol-d₄) δ 9.19-8.90 (d, J=4.5 Hz, 1H), 8.79-8.45 (s, J=2.3 Hz, 1H), 8.09-8.02 (m, 2H), 8.01-7.91 (m, 2H), 7.84-7.75 (dd, J=8.1, 0.9 Hz, 1H), 7.63-7.52 (d, J=4.4 Hz, 1H), 4.85 (s, 2H).

### PI3Ki1

Compound 5 (136 mg, 0.32 mmol) and compound 8 (110 mg, 0.32 mmol) were dissolved in anhydrous 1.4-dioxane (50 mL). To this was added Pd(dppf)₂Cl₂ (10 mg, 0.012 mmol) followed by 2M Na₂CO₃ (8 mL). The mixture was then heated at reflux for 6 hrs. After cooling to room temperature, the solids were filtered off and the residue evaporated. The crude product was purified by flash chromatography (EtOAc:MeOH) to give PI3Ki1 (65% yield). LRMS-LCMS (m/z): [M+H]⁺ calcd for C₂₇H₂₀F₂N₄O₄S, 534.1; found 535.1). ¹H NMR (500 MHz, DMSO-d₆) δ 10.39-10.23 (s, 1H), 9.08-8.88 (d, J=4.4 Hz, 1H), 8.84-8.67 (d, J=2.2 Hz, 1H), 8.39-8.28 (d, J=2.4 Hz, 1H), 8.28-8.18 (d, J=8.7 Hz, 1H), 8.18-8.11 (dd, J=8.0, 2.3 Hz, 1H), 8.10-8.00 (dd, J=8.7, 2.1 Hz, 1H), 7.97-7.91 (d, J=2.1 Hz, 1H), 7.91-7.82 (s, 1H), 7.77-7.68 (dq, J=6.3, 5.1, 4.0 Hz, 2H), 7.63-7.45 (m, 2H), 7.22-7.09 (td, L=8.5, 2.5 Hz, 1H), 5.70-5.44 (t, J=5.9 Hz, 1H), 4.79-4.54 (d, J=5.8 Hz, 2H), 3.64 (s, 3H).

### Compound 11

To a solution of compound 9 in DMF compound 10 (1 eq) and HATU (1 eq) were added. To the above solution, anhydrous DIPEA (5 eq) was added and stirred under argon atmosphere for 6 h. The crude product was purified using RP-HPLC [A=2 Mm ammonium acetate buffer (pH 7.0), B=acetonitrile, solvent gradient 0% B to 80% B in 35 min] to yield compound 11 (70% yield). LRMS-LCMS (m/z): [M+H]+ calcd for C₁₃H₂₁F₂N₃O₄, 321.32; found 322, 266, and 222. ¹H NMR (500 MHz, Chloroform-d) δ 6.69 (s, 1H), 5.74 (s, 1H), 5.26 (d, J=7.8 Hz, 1H), 4.81 (dd, J=9.3, 5.4 Hz, 1H), 4.40 (p, J=7.1 Hz, 1H), 4.25-4.09 (m, 1H), 3.88-3.70 (m, 1H), 3.08-2.85 (m, 1H), 2.62-2.48 (m, 1H), 1.42 (s, J=10.7 Hz, 9H), 1.32 (d, J=8.7, 7.0 Hz, 3H).

### Compound 12

The HPLC purified compound 11 was dissolved in DMF. To this solution were added anhydrous pyridine (1 eq) and TFAA (1 eq). The reaction mixture was stirred at room temperature for 1 h. Completion of the reaction was monitored by LCMS. The crude product was purified using RP-HPLC [A=2 Mm ammonium acetate buffer (pH 7.0), B=acetonitrile, solvent gradient 0% B to 80% B in 35 min] to yield compound 12 (75% yield). LRMS-LCMS (m/z): [M+H]+ calcd for C₁₃H₁₉F₂N₃O₃, 303.31; found 305, 248, and 204. ¹H NMR (500 MHz, Chloroform-d) δ 5.18 (d, J=8.3 Hz, 1H), 5.00 (dd, J=7.8, 5.3 Hz, 1H), 4.31 (p, J=7.2 Hz, 1H), 4.19 (dt, J=16.0, 11.0 Hz, 1H), 3.94 (td, J=12.2, 8.7 Hz, 1H), 2.84-2.69 (m, 2H), 1.42 (s, 9H), 1.35 (d, J=7.0 Hz, 3H).

### Compound 14

Compound 12 was dissolved in TFA followed by stirring at room temperature for 30 min. The completion of the reaction was monitored through LCMS. This compound was dried under high vacuum and used further without any purification. To the TFA solution of compound 12, compound 13 (1 eq) and HATU (1 eq) in DMF and DIPEA (5 eq) were added and stirred under argon atmosphere for 6 h. The completion of the reaction was monitored by LCMS. The crude material was purified using RP-HPLC [A=2 Mm ammonium acetate buffer (pH 7.0), B=acetonitrile, solvent gradient 0% B to 80% B in 35 min] to yield compound 14 (80% yield). LRMS-LCMS (m/z): [M+H]+ calcd for C₂₀H₂₅F₂N₅O₄, 437.45; found 438. ¹H NMR (500 MHz, Chloroform-d) δ 8.58 (d, J=5.2 Hz, 1H), 7.55 (s, 1H), 7.50 (s, 1H), 7.42 (d, J=4.9 Hz, 1H), 5.55 (s, 1H), 5.11 (s, 1H), 4.75 (p, J=7.1 Hz, 1H), 4.49-4.38 (m, 2H), 4.29 (dt, J=16.2, 10.7 Hz, 1H), 4.04 (td, J=12.2, 11.7, 7.8 Hz, 1H), 2.89-2.77 (m, 2H), 1.53 (d, J=7.1 Hz, 3H), 1.46 (s, 9H).

### FAPL

Compound 14 was dissolved in TFA followed by stirring at room temperature for 30 min. TFA was removed, and the crude compound was used for the next reaction without any further purification. The crude compound from TFA deprotection was dissolved in DMF and to this mixture compound 15 (1 eq), HATU (1 eq) and DIPEA (10 eq) were added and stirred under argon atmosphere for 6 h. The completion of reaction was monitored by LCMS. The crude material was purified by using RP-HPLC [A=2 Mm ammonium acetate buffer (pH 7.0), B=acetonitrile, solvent gradient 0% B to 80% B in 35 min] to yield FAPL (65% yield). LRMS-LCMS (m/z): [M+H]+ calcd for C₁₉H₂₁F₂N₅O₅, 437.4; found 438. ¹H NMR (500 MHz, Deuterium Oxide) δ 8.58-8.47 (d, J=4.8 Hz, 1H), 7.67-7.40 (m, 2H), 5.10-5.02 (dd, J=9.1, 4.3 Hz, 1H), 4.64-4.54 (q, J=7.2 Hz, 1H), 4.45 (s, 2H), 4.22-4.13 (m, 2H), 3.05-2.70 (m, 2H), 2.55 (s, 4H), 1.43-1.33 (d, J=1 Hz, 3H).

### Compound 16

Compound 16 was prepared by solid phase peptide coupling conditions with HATU and DIPEA using H-Cys(Trt)-2-Cl-Trt. The final product was cleaved from the resin using the standard cocktail solution of TFA:Water:TIPS:Ethanedithiol (95%:2.5%:2.5%:2.5%), The crude compound was precipitated in ether to yield compound 16 (45% yield), and was used without further purification. LRMS-LCMS (m/z): [M+H]+ calcd for C₂₂H₂₆F₆N₆O₆S, 540.54; found 541. ¹H NMR (500 MHz, Methanol-d₄) δ 8.61 (d, J=5.1 Hz, 1H), 7.77 (s, 1H), 7.69-7.57 (m, 2H), 5.11 (dd, J=9.4, 3.4 Hz, 1H), 4.67 (q, J=7.1 Hz, 1H), 4.55 (s, 1H), 4.53 (s, 1H), 4.35 (t, J=5.1 Hz, 2H), 4.34-4.16 (m, 2H), 2.96-2.82 (m, 3H), 2.82-2.72 (m, 1H), 2.71-2.56 (m, 6H), 1.50 (s, 3H), 1.48 (s, 1H),

### Compound 18

PI3Ki1 (50 mg, 0.094 mmol) and compound 17 (32.7 mg, 0.094 mmol) were dissolved in DMF (1 mL) and stirred. Progress of the reaction was monitored by analytical LCMS. Following completion of the reaction, crude product was purified by preparative RP-HPLC [A=2 mM ammonium acetate buffer (pH 7.0), B=acetonitrile, solvent gradient 5% B to 80% B in 35 min] to yield 18 (45% yield). LRMS-LCMS (m/z): [M+H]⁺ calcd for C₃₅H₂₇F₂N₅O₆S₃, 747.1; found 748.1). ¹H NMR (500 MHz, DMSO-d₆) δ 9.05-8.93 (d, J=4.4 Hz, 1H), 8.86-8.74 (d, J=2.2 Hz, 1H), 8.72-8.62 (d, J=2.3 Hz, 1H), 8.41-8.31 (d, J=4.8 Hz, 1H), 8.27-8.20 (m, 2H), 8.20-8.10 (m, 3H), 8.10-8.02 (d, J=2.1 Hz, 1H), 7.77-7.62 (m, 3H), 7.62-7.55 (m, 2H), 7.45-7.36 (t, J=7.8 Hz, 1H), 7.21-7.08 (dd, J=7.4, 4.8 Hz, 1H), 5.62 (s, 1H), 4.74 (s, 2H), 4.36-4.17 (d, J=6.0 Hz, 2H), 3.88 (s, 3H), 2.95-2.86 (t, J=5.9 Hz, 2H).

### FAPL-PI3Ki1

Compound 18 (22.3 mg, 0.019) and compound 16 (10 mg, 0.018 mmol) were dissolved in anhydrous DMSO and stirred under inert atmosphere. Progress of the reaction was monitored by analytical LCMS. Following completion of the reaction, crude product was purified by preparative RP-HPLC [A=2 mM ammonium acetate buffer (pH 7.0), B=acetonitrile, solvent gradient 5% B to 80% B in 35 min] to afford the final product FAPL-PI3Ki1 (34% yield). LRMS-LCMS (m/z): [M+H]³⁰ calcd for C₅₂H₄₈F₄N₁₀O₁₂S₃, 1177.2 found 1179.1)

### FAPL-Fluorescein

Compound 16 (1 eq) and Mal-Fluorescein (1 eq) were dissolved in anhydrous DMF containing DIPEA (1 eq) and stirred under inert atmosphere. Progress of the reaction was monitored by analytical LCMS. Following completion of the reaction; crude product was purified by preparative RP-HPLC [A=2 mM ammonium acetate buffer (pH 7.0), B=acetonitrile, solvent gradient 5% B to 80% B in 35 min] to afford the final product FAPL-fluorescein (65% yield). LRMS-LCMS (m/z): [M+H]⁺ calcd for C₄₆H₄₀F₂N₇O₁₃S⁻, 968.24 found 968)

### FAPL-S0456

Compound 16 (1 eq) and Mal-S0456 (1 eq) were dissolved in anhydrous DMSO containing DIPEA (1 eq) and stirred under inert atmosphere. Progress of the reaction was monitored by analytical LCMS. Following completion of the reaction; crude product was purified by preparative RP-HPLC [A=2 mM ammonium acetate buffer (pH 7.0), B=acetonitrile, solvent gradient 5% B to 80% B in 35 min] to afford the final product FAPL-S0456 (70% yield). LRMS-LCMS (m/z): [M+H]⁺ calcd for C₇₅H₈₈F₂N₁₀O₂₂S₅, 1678.46 found 1679)

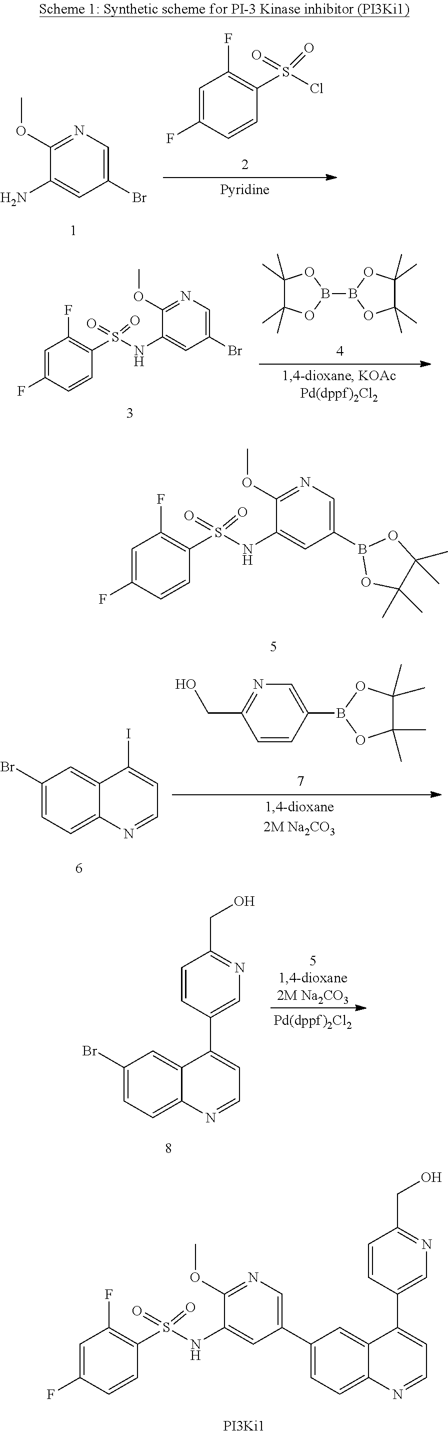

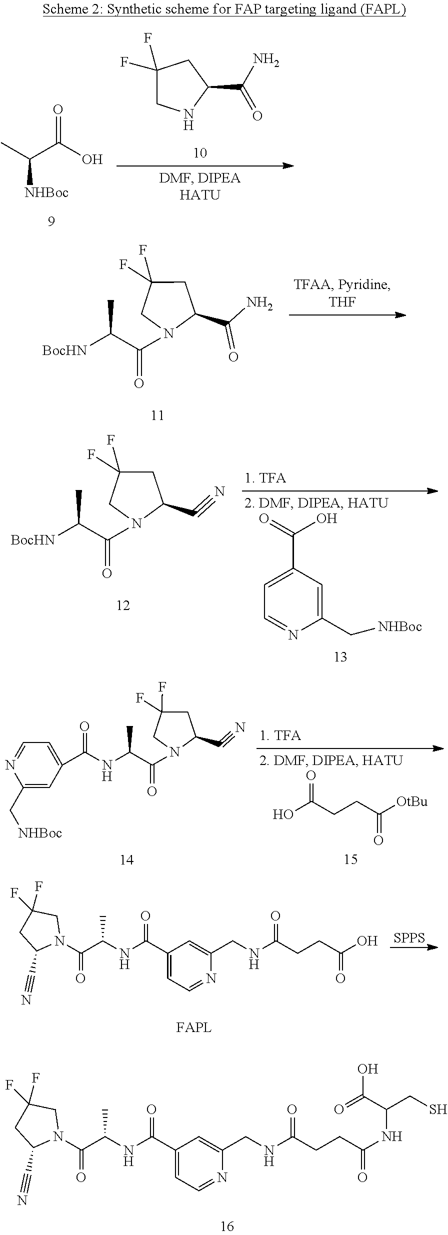

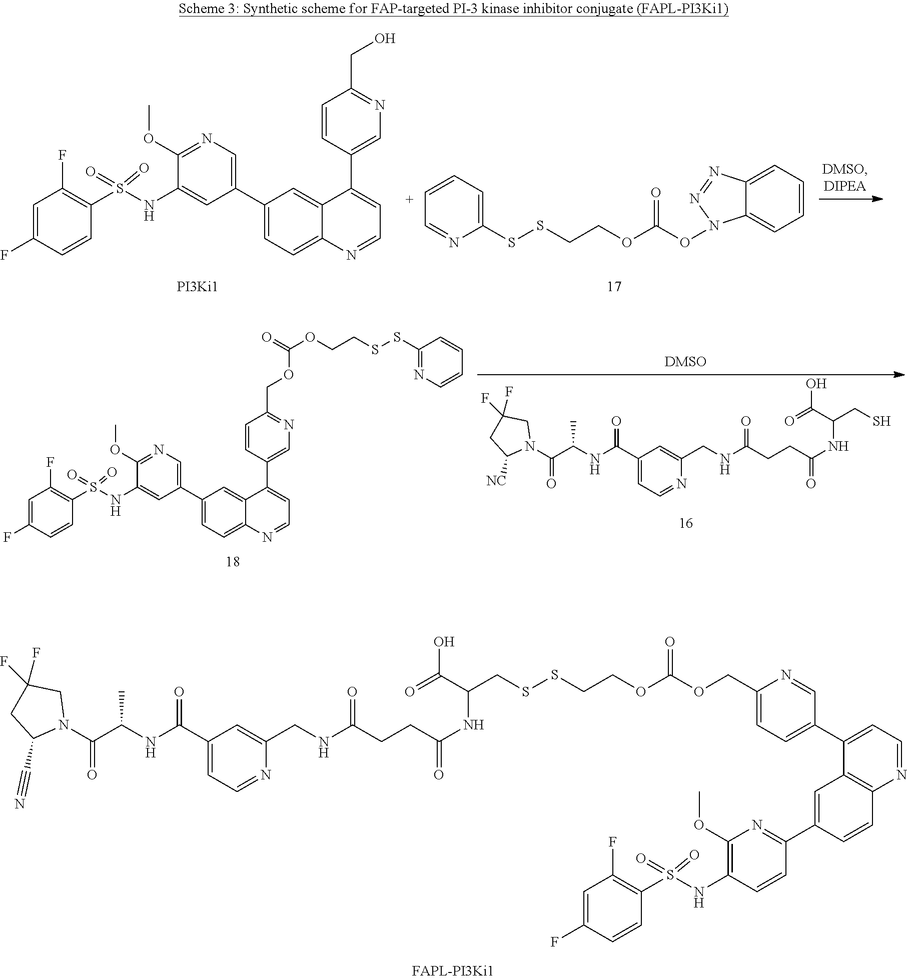

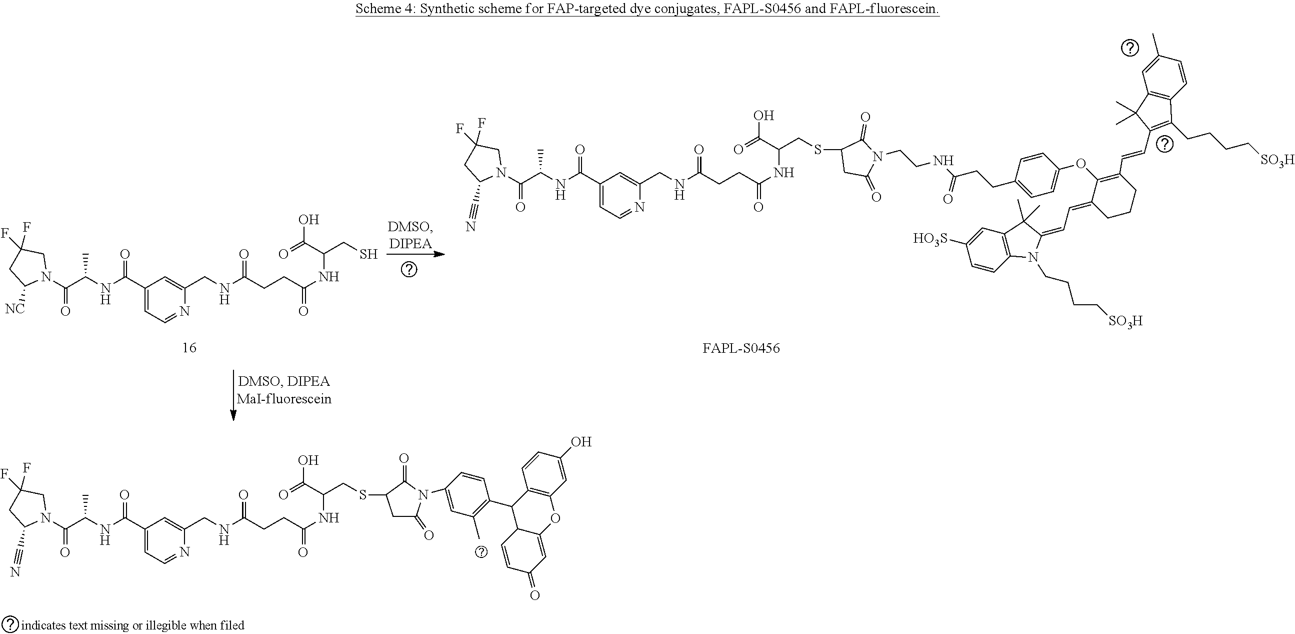 [SMILES: C[C@H](NC(=O)c1ccnc(CNC(=O)CCC(=O)NC(CS)C(=O)O)c1)C(=O)N1CC(F)(F)C[C@H]1C#N ; Cc1ccc2c(c1)C(C)(C)C(/C=C/C1=C(Oc3ccc(CCC(=O)NCCN4C(=O)CC(SCC(NC(=O)CCC(=O)NCc5cc(C(=O)N[C@@H](C)C(=O)N6CC(F)(F)C[C@H]6C#N)ccn5)C(=O)O)C4=O)cc3)C(=C/C=C3/N(CCCCS(=O)(=O)O)c4ccc(S(=O)(=O)O)cc4C3(C)C)/CCC1)=C2CCCCS(=O)(=O)O ; Cc1cc(N2C(=O)CC(SCC(NC(=O)CCC(=O)NCc3cc(C(=O)N[C@@H](C)C(=O)N4CC(F)(F)C[C@H]4C#N)ccn3)C(=O)O)C2=O)ccc1C1c2ccc(O)cc2OC2=CC(=O)C=CC21]

1. A compound of the formula (II): 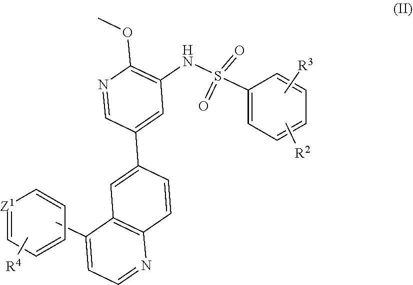
or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R⁴ is a group of the formula D-L-O-alkyl-, D-L-N(R^{e})-alkyl-, D-L-S(O)ₓalkyl, D-L-C(O)—, or D-L-C(O)-alkyl, wherein L is a linker, and D is a fibroblast activation protein (FAP) ligand; and
R² and R³ are each, independently, H, halo, hydroxy, alkyl, alkoxy, aryl, amino, acyl or C(O)R^{b}, wherein R^{b} is alkyl, aryl, OH or alkoxy.

2. The compound of claim 1, wherein the compound is a compound of the formula: 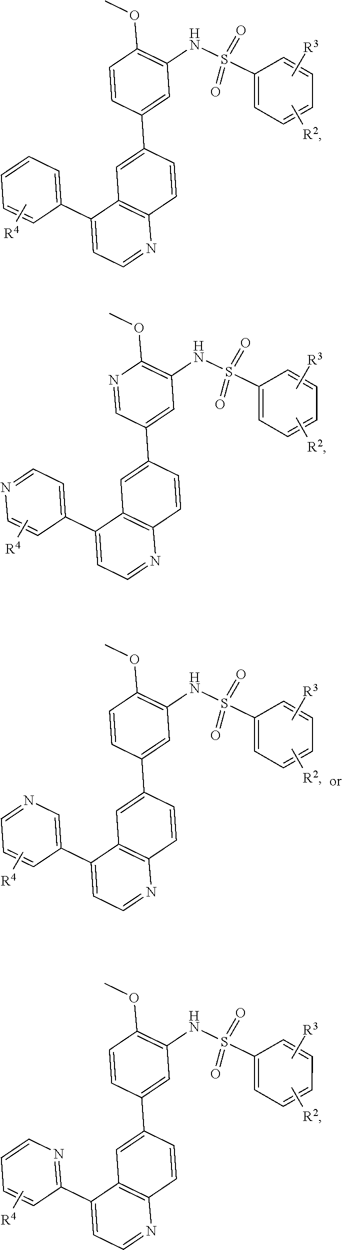
or a pharmaceutically acceptable salt thereof.

3. The compound of claim 1, wherein the compound is a compound of the formula: 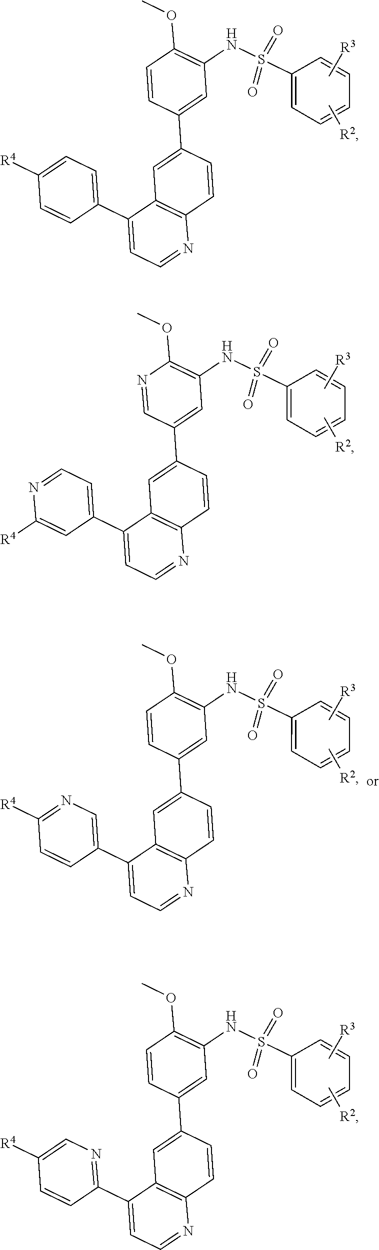
or a pharmaceutically acceptable salt thereof.

4. The compound of claim 3, wherein L is a hydrolyzable linker.

5. The compound of claim 3, wherein L is an optionally substituted heteroalkyl.

6. The compound of claim 5, wherein the substituted heteroalkyl is substituted with at least one substituent selected from the group consisting of alkyl, hydroxyl, acyl, polyethylene glycol (PEG), carboxylate, and halo.

7. The compound of claim 1, wherein L is a substituted heteroalkyl with at least one disulfide bond in the backbone thereof.

8. The compound of claim 1, wherein L is a peptide or a peptidoglycan with at least one disulfide bond in the backbone thereof.

9. The compound of claim 1, wherein L has the formula:
—CO—(CH₂)₂—CONH—CH(COOH)—CH₂—CR⁶R⁷—S—S—CH₂—O—CO—,
wherein R⁶ and R⁷ are each, independently, H, alkyl, or heteroalkyl.

10. The compound of claim 1, wherein L is a group or comprises a group of the formula: 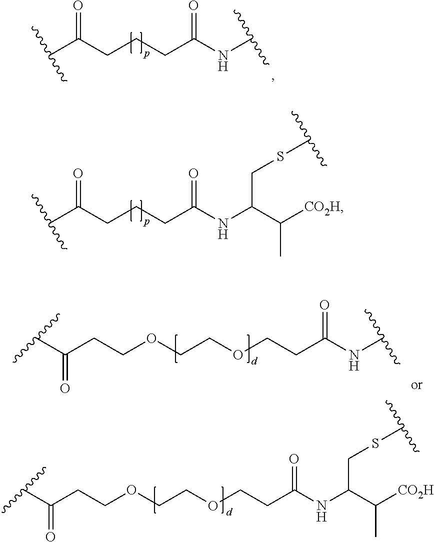
wherein p is an integer from 0 to 10; and d is an integer from 1 to 40.

11. The compound of claim 1, wherein D is a group or comprises a group of the formula (III):

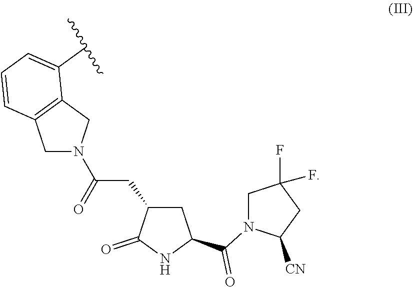 [SMILES: *c1cccc2c1CN(C(=O)C[C@@H]1C[C@@H](C(=O)N3CC(F)(F)C[C@H]3C#N)NC1=O)C2]

12. The compound of claim 1, wherein D is a group or comprises a group of the formula (IV): 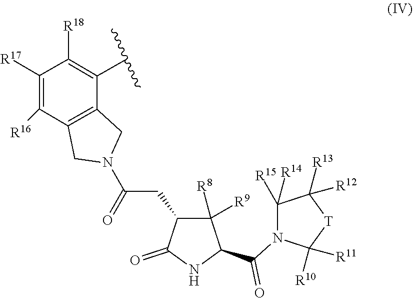
wherein,
T is CH₂, NH, O or S;
R¹⁰ and R¹¹ are each, independently, —H, —CN, —CHO, —B(OH)₂, —C(O)alkyl, —C(O)aryl, —C═C—C(O)aryl, —C═C—S(O)₂aryl, —CO₂H, —SO₃H, —SO₂NH₂, —PO₃H₂, —SO₂F or 5-tetrazolyl;
R¹² and R¹³ are each, independently, —H, —OH, F, Cl, Br, I, —C₁₋₆alkyl, —O—C₁₋₆alkyl, or —S—C₁₋₆alkyl;
R⁸, R⁹, R,¹⁴, and R¹⁵ are each, independently, H, alkyl or halo; and
R¹⁶-R¹⁸ are each, independently, H, —C₁₋₆alkyl, —O—C₁₋₆alkyl, —S—C₁₋₆ alkyl, F, Cl, Br, or I.

13. The compound of claim 1, wherein D is a group or comprises a group of the formula (IV): 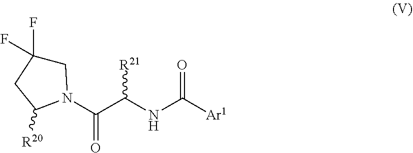
wherein,
R²⁰ is —H, —CN, —B(OH)₂, —C(O)alkyl, —C(O)aryl, —C═C—C(O) aryl, —C═C—S(O)₂aryl, —CO₂H, —SO₃H, —SO₂NH₂, —PO₃H₂, or 5-tetrazolyl;
R²¹ is H or CH₃; and
Ar¹ is substituted phenyl, pyridyl, chloropyridyl, or quinolinyl.

14. The compound of claim 1, wherein the compound of the formula (II) is a compound of the formula: 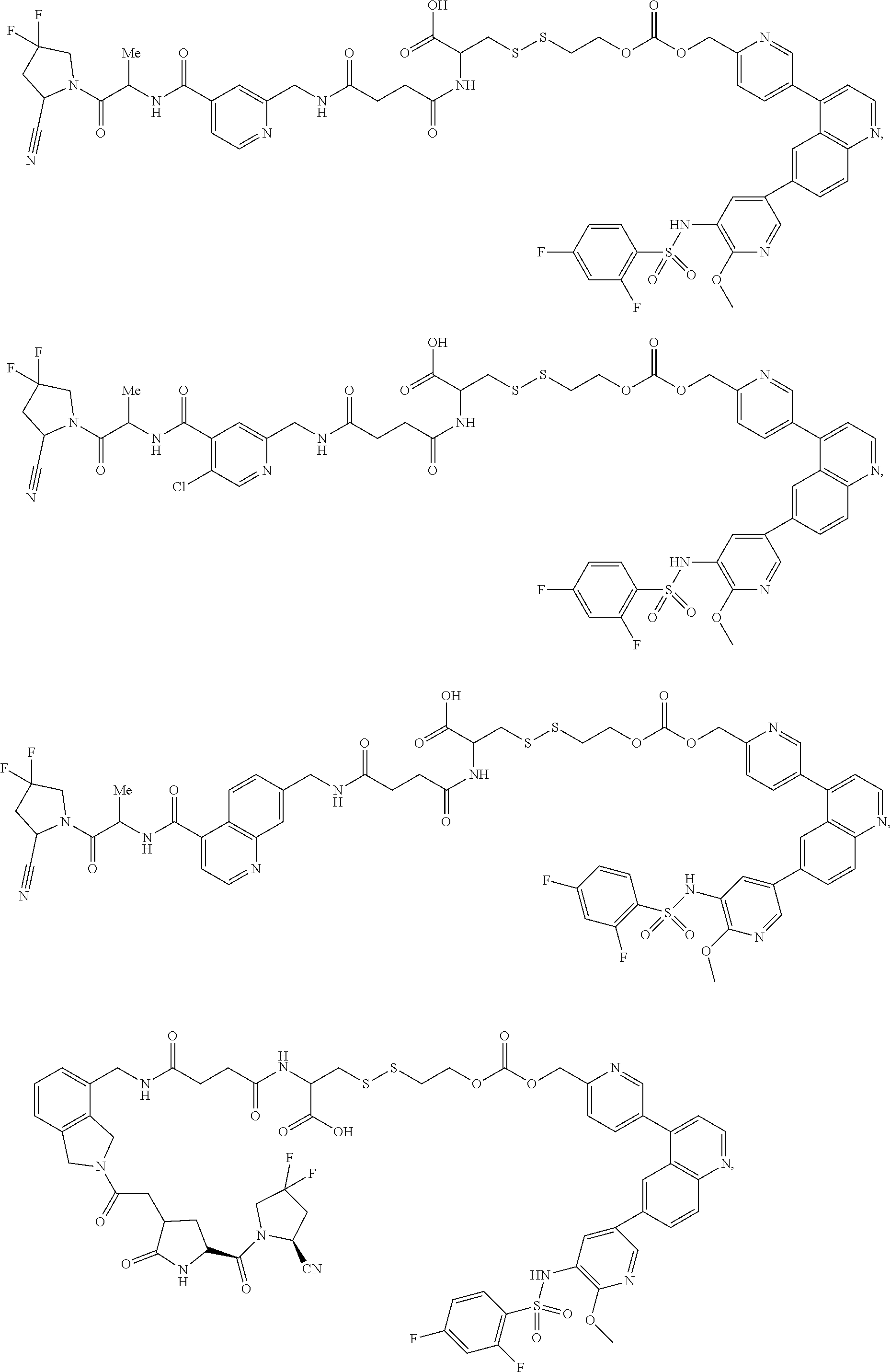 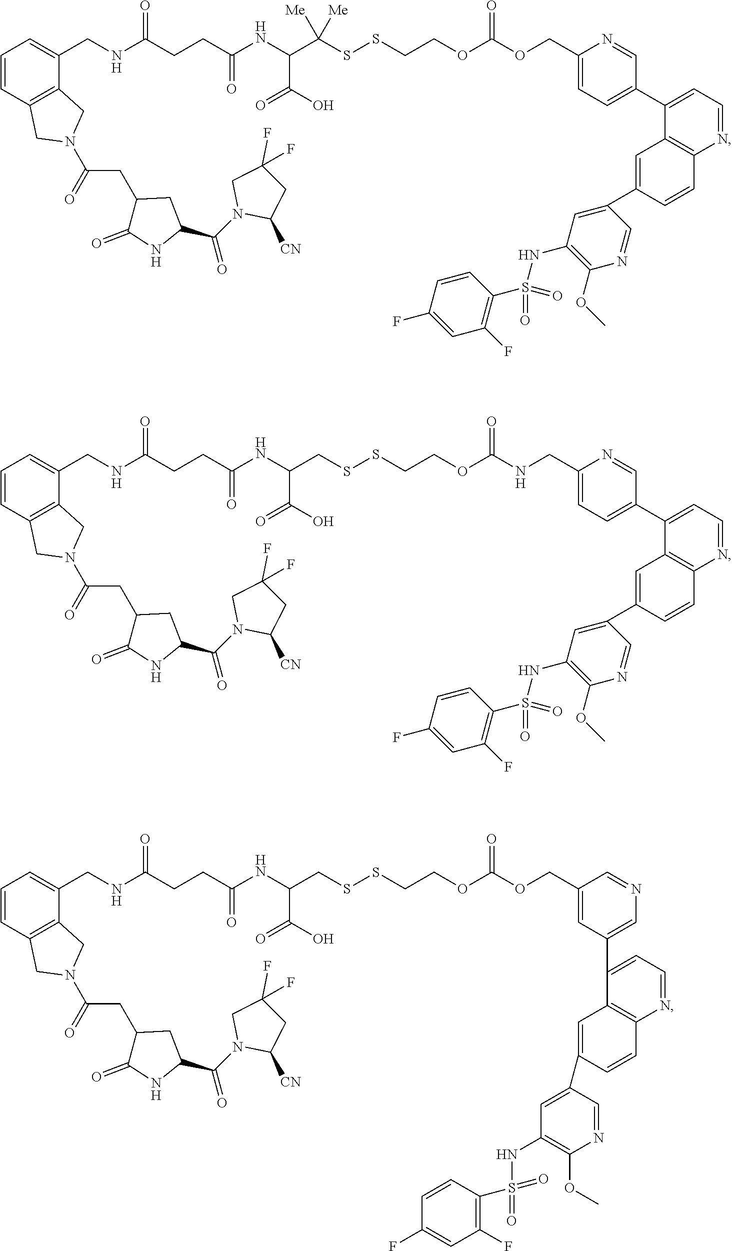 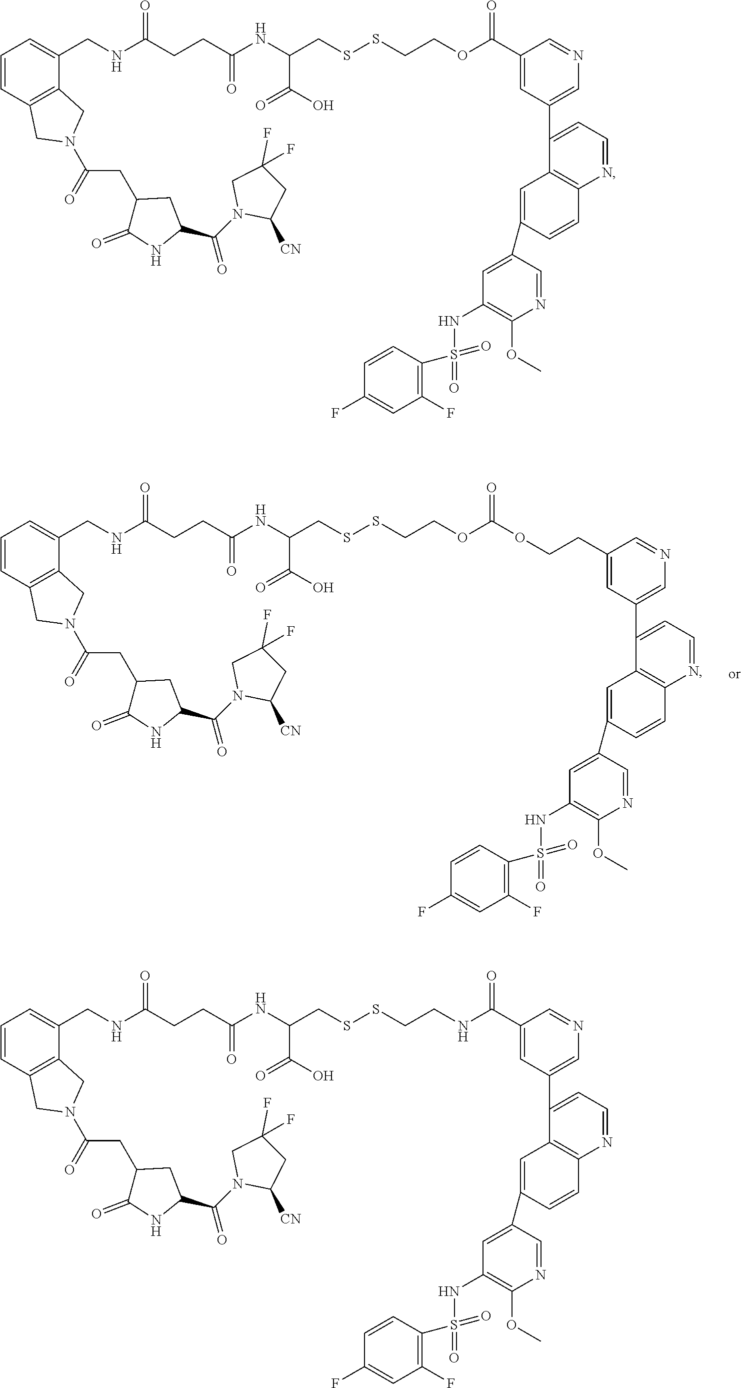
or a pharmaceutically acceptable salt thereof.

15. A pharmaceutical composition comprising a therapeutically effective amount of one or more compounds of claim 1 and at least one pharmaceutically acceptable excipient.

16. A method for treating fibrosis, the method comprising administering a therapeutically effective amount of one or more compounds of claim 1.

17. A compound of the formula (I): 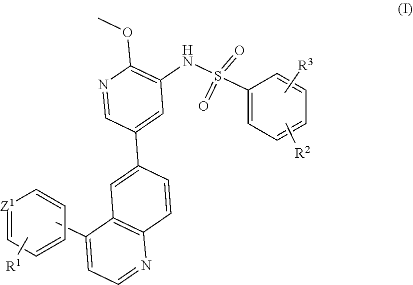
or a pharmaceutically acceptable salt thereof wherein:
Z¹ is CR^{a} or N, wherein R^{a} is H, halo, hydroxy, alkyl, alkoxy, aryl, amino, acyl or C(O)R^{b}, wherein R^{b} is alkyl, aryl, OH or alkoxy;
R¹ is hydroxyalkyl, aminoalkyl, —S(O)ₓalkyl (wherein x is 0, 1 or 2), carboxyl, carboxylalkyl, thiocarboxyl, thiocarboxylalkyl, amino or amidoalkyl;
R² and R³ are each, independently, H, halo, hydroxy, alkyl, alkoxy, aryl, amino, acyl or C(O)R^{b}, wherein R^{b} is alkyl, aryl, OH or alkoxy.

18. The compound of claim 17, wherein the compound is a compound of the formula: 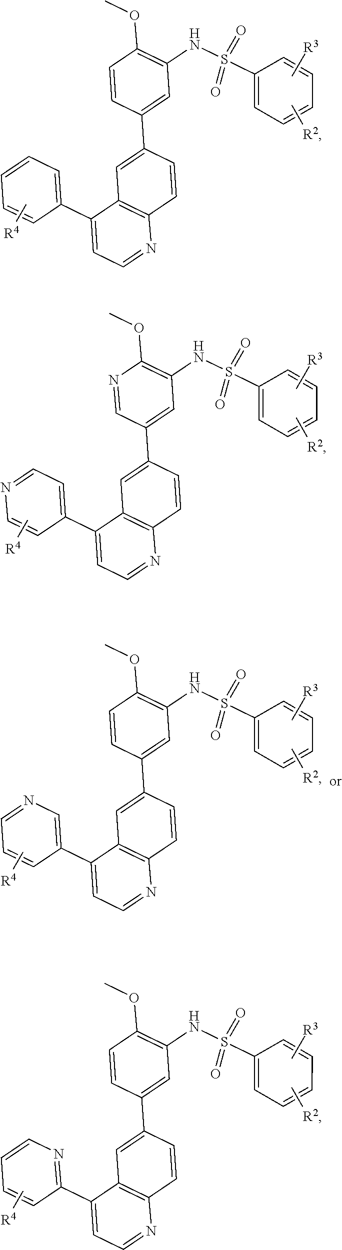
or a pharmaceutically acceptable salt thereof.

19. The compound of claim 17, wherein the compound is a compound of the formula: 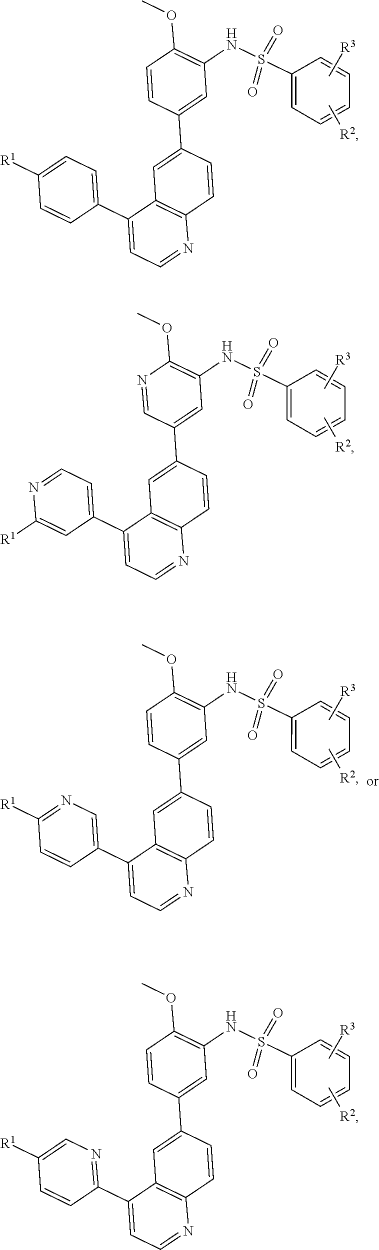
or a pharmaceutically acceptable salt thereof.

20. The compound of claim 17 wherein R¹ is a group of the formula R^{c}O-alkyl-, wherein R^{c} is H or a hydroxyl protecting group; (R^{d})₂N-alkyl-, wherein R^{d} is H or an amine protecting group; R^{e}S(O)ₓ-alkyl-; R^{e}O(O)C—; R^{e}O(O)C-alkyl-; R^{e}S(O)C—; R^{e}S(O)C-alkyl-; (R^{e})₂N(O)C—; or (R^{e})₂N(O)C-alkyl-; wherein R^{e} is H or alkyl, and x is 0, 1, or 2.

21. The compound of claim 17, wherein R¹ is a group of the formula R^{c}O(CH₂)ₙ—, wherein R^{c} is H or a hydroxyl protecting group; (R^{d})₂N(CH₂)ₙ—, wherein R^{d} is H or an amine protecting group; R^{e}S(O)ₓ(CH₂)ₙ—, wherein R^{e} is H or alkyl, and x is 0, 1, or 2; R^{e}O(O)C(CH₂)ₙ—, wherein R^{e} is H or alkyl; R^{e}S(O)C—, wherein R^{e} is H or alkyl; R^{e}S(O)C(CH₂)ₙ—, wherein R^{e} is H or alkyl; or (R^{e})₂N(O)C(CH₂)ₙ—, wherein R^{e} is H or alkyl; wherein n is an integer from 1 to 20.

22. The compound of claim 17, wherein the compound is a compound of the formula: 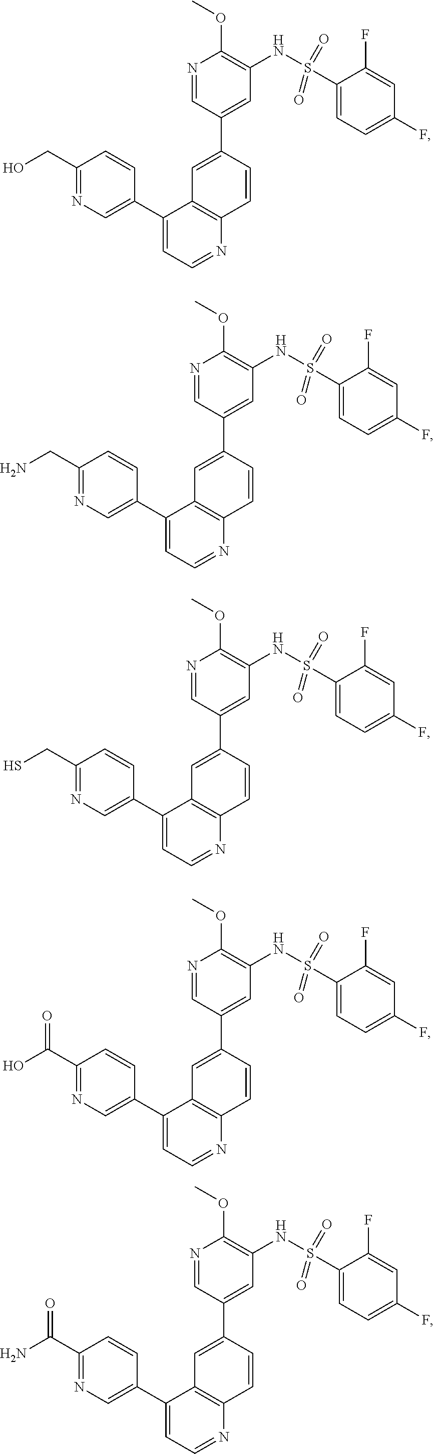 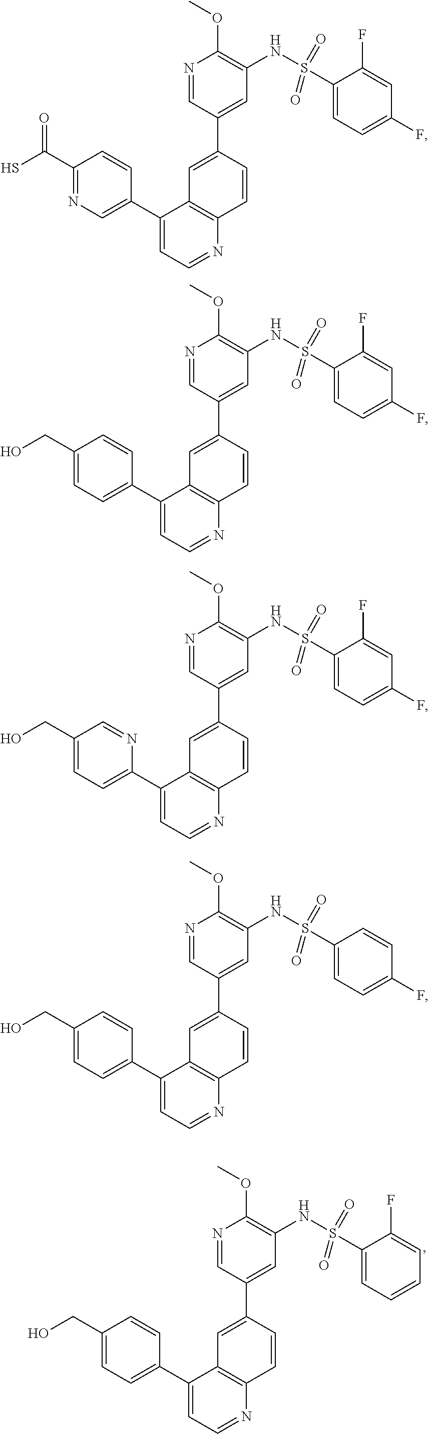 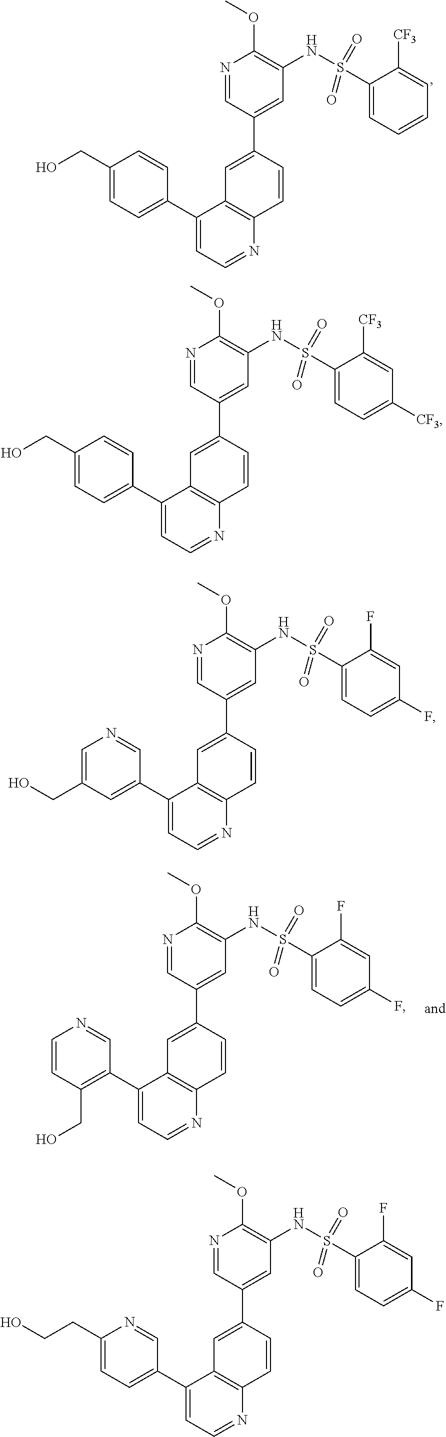
or a pharmaceutically acceptable salt thereof.


SEQUENCE LISTING (12 sequences)

SEQ ID NO: 1 - DNA, 23 bp, Artificial Sequence
  A synthetic sequence
gcttaatttgactcaacacggga

SEQ ID NO: 2 - DNA, 24 bp, Artificial Sequence
  A synthetic sequence
agctatcaatctgtcaatcctgtc

SEQ ID NO: 3 - DNA, 21 bp, Artificial Sequence
  A synthetic sequence
cagggctgttttcccatccat

SEQ ID NO: 4 - DNA, 22 bp, Artificial Sequence
  A synthetic sequence
gccatgttctatcgggtacttc

SEQ ID NO: 5 - DNA, 20 bp, Artificial Sequence
  A synthetic sequence
agccagcagatcgagaacat

SEQ ID NO: 6 - DNA, 20 bp, Artificial Sequence
  A synthetic sequence
tccttggggttcttgctgat

SEQ ID NO: 7 - DNA, 20 bp, Artificial Sequence
  A synthetic sequence
actgtacatgcttcggtcag

SEQ ID NO: 8 - DNA, 21 bp, Artificial Sequence
  A synthetic sequence
agtctctgaatcctggcattg

SEQ ID NO: 9 - DNA, 18 bp, Artificial Sequence
  A synthetic sequence
ggtgaacgtggtgcagct

SEQ ID NO: 10 - DNA, 22 bp, Artificial Sequence
  A synthetic sequence
tctttaccaggagaaccatcag

SEQ ID NO: 11 - DNA, 21 bp, Artificial Sequence
  A synthetic sequence
ctttggcagtggtcatttcag

SEQ ID NO: 12 - DNA, 20 bp, Artificial Sequence
  A synthetic sequence
attctccctttccattcccg